(19) United States
(12) Patent Application Publication
(10) Pub. No.: US 2011/0230497 A1
(43) Pub. Date: Sep. 22, 2011

(54) BIOLOGICALLY ACTIVE AMIDES

(75) Inventors: John Matthew Peterson, Slate Hill, NY (US); Chien-An Chen, Flushing, NY (US); Bin Chen, East Windsor, NJ (US); Lingyun Wu, New Milford, NJ (US); Michael Lloyd Sabio, Ridgewood, NJ (US)

(73) Assignee: H. Lundbeck A/S, Valby-Copenhagen (DK)

(21) Appl. No.: 13/127,099

(22) PCT Filed: Nov. 2, 2009
(86) PCT No.: PCT/US09/62956

§ 371 Date: Jun. 8, 2011

Related U.S. Application Data
(60) Provisional application No. 61/161,854, filed on Mar. 20, 2009.
(60) Provisional application No. 61/112,333, filed on Nov. 7, 2008.

(51) Int. Cl. A61K 31/497; C07D 471/04; A61K 31/437; A61P 25/00; C07D 401/12; A61K 31/4545; C07D 498/04; A61K 31/4375; A61K 31/506; C07D 211/34; A61K 31/445

(52) U.S. Cl. 514/255.05; 546/183; 514/299; 546/194; 514/318; 546/116; 514/302; 544/336; 544/332; 514/275; 546/226; 514/330

(57) ABSTRACT

The present invention is directed to biologically active amides which are ligands at the NPY Y5 receptor. The invention further provides a pharmaceutical composition comprising a therapeutically effective amount of a compound of the invention and a pharmaceutically acceptable carrier. This invention also provides a method of treating a subject suffering from certain disorders which comprises administering to the subject an amount of a compound of the subject invention. Furthermore, this invention also provides uses of a compound of the invention for the manufacture of a medicament for treating a subject suffering from certain disorders.

### FIELD OF THE INVENTION

The present invention relates to compounds which are ligands at the neuropeptide Y Y5 receptor, and as such are useful to treat disorders related to mood, stress, cognition, stress and dementia.

### BACKGROUND OF THE INVENTION

Neuropeptide Y (NPY) is a 36 amino acid neuropeptide expressed in the peripheral and central nervous system. This peptide is a member of the pancreatic polypeptide family, which also includes pancreatic polypeptide (PP) and peptide YY (PYY). Moreover, the biological effects of NPY are mediated through its interaction with receptors that belong in the superfamily of G protein-coupled receptors.

Presently, five NPY receptor subtypes have been cloned: Y1 (D. Larhammar, et al., J. Biol. Chem., 1992, 267, 10935-10938); Y2 (C. Gerald, et al., J. Biol. Chem., 1995, 270, 26758-26761); Y4 (J. Bard, et al., J. Biol. Chem., 1995, 270, 26762-26765); Y5 (C. Gerald, et al., J. Biol. Chem., 1995, 270, 26758-26761); and y6 (P. Gregor, et al., J. Biol. Chem., 1996, 271, 27776-27781). All these receptor subtypes are expressed in several species except for the y6 subtype, which has been shown to be expressed in mouse and rabbit but not in rat and primate. A Y3 subtype has been proposed based on pharmacological data. However, the Y3 subtype has yet to be cloned and its existence remains to be fully established.

NPY exerts numerous physiological effects. On the basis of animal studies, it is evident that a contributory relationship exists between NPY and its receptors with disorders such as depression, anxiety and obesity. For instance, NPY expression is shown to be sensitive to energy status while NPY administration reduces energy expenditure. Another significant ability of NPY is to acutely stimulate feeding (S. Kalra, et al., Endocr. Rev., 1999, 20, 68-100). The NPY Y5 receptor has also been shown to be a receptor subtype responsible for NPY-induced food intake (C. Gerald, et al., Nature, 1996, 382, 168-171).

The link between NPY and mood disorders such as depression and anxiety is established in the literature. For example, rats subjected to chronic mild stress exhibit anhedonia, a feature of clinical depression (P. Willner, et al., Eur. J. Pharmacol., 1997, 340, 121-132); they also contain elevated levels of NPY mRNA in hypothalamus accompanied by a reduction in hippocampus (V. Sergeyev, et al., Psychopharmacology, 2005, 178, 115-124). The behavioral changes associated with chronic mild stress are reversed by a variety of antidepressants (P. Willner, et al., Eur. J. Pharmacol., 1997, 340, 121-132). In one study of antidepressant therapies, rats treated with citalopram displayed an increased level of hippocampal NPY receptor binding with no change in NPY-like immunoreactivity (H. Husum, et al., Neuropsychopharmacology, 2001, 2, 183-191); conversely, electroconvulsive shock produced an increased level of hippocampal NPY-like immunoreactivity with no change in NPY receptor binding. These findings suggest that abnormal levels of NPY play a role in depressive illness, and that agents capable of regulating NPY and/or NPY receptor function particularly in limbic regions are useful for treating depression. Y5 is a NPY receptor expressed in limbic regions (M. Wolak, et al., J. Comp. Neurol., 2003, 22, 285-311; and K. Nichol, et al., J. Neurosci., 1999, 19, 10295-10304). Accordingly, agents capable of regulating Y5 receptor function are therefore predicted to be useful for treating depression.

Animal models of anxiety also reveal abnormal levels of NPY. In one example, maternally separated rats display an anxious and depressive phenotype throughout adulthood (R. Huot, Psychopharmacology, 2001, 158, 366-73); they also contain elevated levels of NPY-like immunoreactivity in hypothalamus accompanied by a reduction in hippocampus and cortex (P. Jimenez-Vasquez, Brain Res. Dev., 2001, 26, 149-152; H. Husum and A. Mathe, Neuropsychopharmacology, 2002 27:756-64; and H. Husum et al., Neurosci Lett., 2002, 333, 127-130). In a second example, rats subjected to fear conditioning display increased anxiety-like behavior; they also contain elevated levels of NPY in hypothalamus, amygdala and nucleus accumbens accompanied by a reduction in frontal cortex. The behavioral changes produced by fear conditioning can be reversed by treatment with anxiolytic drugs. In one study of fear conditioning, both the anxiety-like behavior and altered expression of NPY were reversed by treatment with diazepam (R. Krysiak, et al., Neuropeptides, 2000, 34, 148-57). These findings further suggest that NPY plays a role in anxiety, and that agents capable of regulating NPY and/or receptor function particularly in limbic regions are useful for treating anxiety. Y5 is a NPY receptor expressed in limbic regions (M. Wolak, et al., J. Comp. Neurol., 2003, 22, 285-311; and K. Nichol, et al., J. Neurosci., 1999, 19, 10295-10304). Accordingly, agents capable of regulating Y5 receptor function are therefore predicted to be useful for treating anxiety.

Several groups disclose the nexus between the NPY Y5 receptor and sleep disorders related to circadian rhythm disruptions. This nexus is based on the discovery that NPY Y5 receptors mediate an important physiologic response in the suprachiasmatic nucleus (SCN) of the hypothalamus in response to the application of NPY. For example, WO 99/05911 and WO 05/30208 disclose this link and propose the use of NPY Y5 receptor ligands for treating sleep disorders. Accordingly, it is expected that the compounds of Formula I can be used for treating sleep disorders, which includes primary insomnia.

The pharmaceutical industry is also targeting NPY Y5 receptor antagonists as potential therapies for the treatment of cognitive impairment/dysfunction disorders. For example, the NPY Y5 receptor antagonist, MK-0557, is currently in clinical trials for the treatment of cognitive impairment in patients with schizophrenia. In support of this indication, WO 03/51356 proposes that NPY Y5 receptor antagonism can be used to treat dementias. Accordingly, it is expected that the compounds of Formula I can used for treating cognitive impairment/dysfunction disorders such as cognitive impairment associated with schizophrenia (CIAS); schizophrenia; dementias; autism; ADHD; and Alzheimer's disease. The compounds of the invention are also expected to treat the positive and negative aspects of schizophrenia; dementias; autism; ADHD; and Alzheimer's disease.

WO 02/28393 discloses methods of reducing self-administration of alcohol in a patient suffering from alcoholism comprising administering a NPY Y5 receptor antagonist. Accordingly, it is expected that the compounds of Formula I can used for treating substance dependency/abuse disorders such as alcoholism as well as nicotine and cocaine addictions.

Furthermore, it is expected that the compounds of Formula I can be used for treating metabolic disorders such as dyslipidemia; hyperlipidemia; insulin hyposensitivity; hyperglycemia; metabolic syndrome; and diabetes mellitus.

NPY expression is shown to be sensitive to energy status while NPY administration reduces energy expenditure, and another significant ability of NPY is to acutely stimulate feeding (S. Kalra, et al., Endocr. Rev., 1999, 20, 68-100). The NPY Y5 receptor has also been shown to be a receptor subtype responsible for NPY-induced food intake (C. Gerald, et al., Nature, 1996, 382, 168-171). Accordingly, it is predicted that the compounds of Formula I can be used for treating eating disorders such as bulimia; bulimia nervosa; binge eating disorder; and night eating disorder.

Analgesic-like effects of NPY have been shown in rats and mice both after intrethecal administration and ICV administration, and after infusion directly into specific brain regions. These studies have used either “spinal” pain models or “supraspinal” models. The involvement of the Y5 receptor subtype has been linked to chronic pain disorders (Woldbye, et al. Brain Research, 2007, 49-55). Accordingly, it is expected that the compounds of Formula I can used for treating chronic pain disorders such as neuropathic pain; neuralgic pain; migraine; fibromyalgia; IBS; chronic fatigue syndrome; chronic tension type headache; chronic low back pain; myofascial pain and chronic osteoarthritis.

Additionally, it is expected the treatment of the cognitive and mood impairment in Parkinson's disease may be an indication target for the compounds of Formula I. The compounds of Formula I may also be used to treat disorders relating to impulsivity and aggression.

### SUMMARY OF THE INVENTION

One objective of the present invention is to provide compounds which are ligands at the NPY Y5 receptor. Accordingly, the present invention relates to compounds of Formula I:

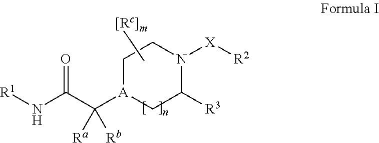

wherein R¹ is phenyl, pyrazoyl, imidazoyl, isoxazoyl, oxadiazoyl, thiadiazoyl, pyridyl, pyrimidyl or pyrazinyl, where the phenyl, pyrazoyl, imidazoyl, isoxazoyl, oxadiazoyl, thiadiazoyl, pyridyl, pyrimidyl and pyrazinyl are optionally substituted with one or more R⁴;
wherein R² is C₁-C₇ alkyl, C₁-C₇ alkoxy, NH(C₁-C₇ alkyl), (CH₂)ᵥOC(O)C₁-C₇ alkyl, C₃-C₇ cycloalkyl, phenyl, pyridyl, pyrimidinyl, pyrazinyl, triazoyl, tetrazolyl, thiazoyl, oxazoyl, imidazoyl, isoxazoyl, furanyl or pyrazoyl, where the phenyl, pyridyl, pyrimidinyl, pyrazinyl, triazoyl, tetrazolyl, thiazoyl, oxazoyl, imidazoyl, isoxazoyl, furanyl and pyrazoyl are optionally substituted with one or more R⁵;
wherein R³ is H or C₁-C₇ alkyl, or where R³ can combine with R² to form C₁-C₄ alkylene optionally substituted with C₁-C₄ alkyl;
wherein R⁴ is C₁-C₇ alkyl, C₁-C₇ perfluoroalkyl, C(O)C₁-C₇ alkyl or phenyl optionally substituted with one or more C₁-C₇ alkyl, C₁-C₇ perfluoroalkyl, C₁-C₇ alkoxy or halogen;
wherein R⁵ is C₁-C₇ alkyl, C₁-C₇ perfluoroalkyl, C₁-C₇ alkoxy or halogen;
wherein A is CH, COH or N;
wherein X is C(O), CO₂ or S(O)₂;
wherein each R^{a} and R^{b} is independently H or C₁-C₇ alkyl, or where R^{a} and R^{b} can combine to form C₃-C₇ cycloalkyl;
wherein each R^{c} is independently H or C₁-C₇ alkyl;
wherein each m and v is independently an integer from 1 to 4 inclusive; and
wherein n is an integer from 0 to 2 inclusive; or a pharmaceutically acceptable salt thereof.

In separate embodiments of the invention, the compound is selected from one of the specific compounds disclosed in the Experimental Section.

Furthermore, the present invention provides a pharmaceutical composition comprising a therapeutically effective amount of a compound of Formula I or a pharmaceutically acceptable salt thereof. The present invention also provides a process for making a pharmaceutical composition comprising admixing a therapeutically effective amount of a compound of Formula I or a pharmaceutically acceptable salt thereof.

The present invention provides a method of treating a subject suffering from mood disorders comprising administering to the subject a therapeutically effective amount of a compound of Formula I. Separately, the present invention further provides a method of treating a subject suffering from anxiety disorders comprising administering to the subject a therapeutically effective amount of a compound of Formula I. The present invention further provides a method of treating a subject suffering from a cognitive disorder comprising administering to the subject a therapeutically effective amount of a compound of Formula I.

Additionally, the present invention is directed to the use of a compound as defined in Formula I for the manufacture of a medicament useful for treating mood disorders. The present invention is directed to the use of a compound as defined in Formula I for the manufacture of a medicament useful for treating anxiety disorders. The present invention further provides for the use of a compound as defined in Formula I for the manufacture of a medicament useful for treating cognitive disorders.

In separate embodiments of the invention, the method or use is selected from one of the specific disorders mentioned in the detailed description of the invention.

### DETAILED DESCRIPTION OF THE INVENTION

In the present invention, the term “C₁-C₇ alkyl” refers to a straight chained or branched saturated hydrocarbon having from one to seven carbon atoms inclusive. Examples of such substituents include, but are not limited to, methyl, ethyl, 1-propyl, 2-propyl, 1-butyl, 2-butyl, 2-methyl-2-propyl, 2-methyl-1-propyl, n-pentyl and n-hexyl. Similarly, the term “straight chained or branched C₁-C₄ alkyl” refers to a saturated hydrocarbon having from one to four carbon atoms inclusive. Examples of such substituents include, but are not limited to, methyl, ethyl and n-butyl.

Likewise, the term “C₁-C₇ alkoxy” refers to a straight chained or branched saturated alkoxy group having from one to seven carbon atoms inclusive with the open valency on the oxygen. Examples of such substituents include, but are not limited to, methoxy, ethoxy, n-butoxy, t-butoxy and n-heptyloxy.

The term “C₃-C₆ cycloalkyl” refers to cyclopropyl, cyclobutyl, cyclopentyl and cyclohexyl.

As used herein, the term “C₁-C₇ perfluoroalkyl” refers to a straight chained or branched saturated hydrocarbon having from one to seven carbon atoms inclusive substituted with one or more fluorine atoms. Examples of such substituents include, but are not limited to, trifluoromethyl, pentafluoroethyl, 1-fluoroethyl, 1,2-difluoroethyl and 3,4 difluoroheptyl. Similarly, the term “straight chained or branched C₁-C₄ fluoroalkyl” refers to a saturated hydrocarbon having from one to four carbon atoms inclusive substituted with one or more fluorine atoms per carbon atom.

As used herein, the term “mood disorders” includes major depressive disorder; minor depressive disorder; dysthymia; cyclothymia; bipolar depression; and depression NUD; and depressive obesity. Moreover, “major depressive disorder” is further divided into melancholic or atypical depression.

As used herein, the term “anxiety disorders” includes panic disorder; agoraphobia; social phobia (aka social anxiety disorder); obsessive compulsive disorder; and generalized anxiety disorder.

As used herein, the term “stress-related disorders” includes acute stress disorder; adjustment disorder; post traumatic stress disorder; exhaustion depression; and stress following (e.g. surgery and fever conditions).

As used herein, the term “sleep disorders” includes primary insomnia and disorders related to disturbances in circadian rhythms.

As used herein, the term “cognitive impairment/dysfunction” includes cognitive impairment associated with schizophrenia; dementias; autism; ADHD; and Alzheimer's disease. Moreover, “dementias” is further divided into age preceding dementia or AIDS dementia.

As used herein, the term “substance dependency/abuse” includes alcohol; nicotine; and cocaine addictions.

As used herein, the term “metabolic disorders” includes dyslipidemia; hyperlipidemia; insulin hyposensitivity; overweight/obesity; hyperglycemia; metabolic syndrome; and diabetes mellitus.

As used herein, the term “chronic pain disorders” include neuropathic pain; neuralgic pain; migraine; fibromyalgia; IBS; chronic fatigue syndrome; chronic tension type headache; chronic low back pain; myofascial pain and chronic osteoarthritis.

A “therapeutically effective amount” of a compound as used herein means an amount sufficient to cure, alleviate or partially arrest the clinical manifestations of a given disease and its complications. An amount adequate to accomplish this is defined as “therapeutically effective amount”. Effective amounts for each purpose will depend on the severity of the disease or injury as well as the weight and general state of the subject. It will be understood that determining an appropriate dosage may be achieved using routine experimentation, by constructing a matrix of values and testing different points in the matrix, which is all within the ordinary skills of a trained physician.

The term “treatment” and “treating” as used herein means the management and care of a patient for the purpose of combating a condition, such as a disease or a disorder. The term is intended to include the full spectrum of treatments for a given condition from which the patient is suffering, such as administration of the active compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of a patient for the purpose of combating the disease, condition, or disorder and includes the administration of the active compounds to prevent the onset of the symptoms or complications. Nonetheless, prophylactic (preventive) and therapeutic (curative) treatment are two separate aspects of the invention. The patient to be treated is a mammal, in particular a human being.

Additionally, aspects of the invention are explained in greater detail below but this description is not intended to be a detailed catalog of all the different ways in which the invention may be implemented, or all the features that may be added to the instant invention. Hence, the following specification is intended to illustrate some embodiments of the invention, and not to exhaustively specify all permutations, combinations and variations thereof.

The invention further provides for the following embodiments:

In an embodiment, X is C(O). In one embodiment, X is CO₂. In another embodiment, X is S(O)₂.

In yet another embodiment, X is CO₂ or S(O)₂.

In another embodiment, R¹ is phenyl optionally substituted with one or more R⁴.

In one embodiment, R¹ is pyrazoyl optionally substituted with one or more R⁴.

In one embodiment, R¹ is [1,3]pyrazoyl optionally substituted with one or more R⁴.

In a separate embodiment, R¹ is imidazoyl optionally substituted with one or more R⁴.

In one embodiment, R¹ is isoxazoyl optionally substituted with one or more R⁴.

In one embodiment, R¹ is [1,3,4]oxadiazoyl optionally substituted with one or more R⁴.

In yet another embodiment, R¹ is [1,3,4]thiadiazoyl optionally substituted with one or more R⁴.

In one embodiment, R¹ is pyridyl optionally substituted with one or more R⁴.

In one embodiment, R¹ is pyrimidinyl or pyrazinyl, where the pyrimidinyl and pyriazinyl is optionally substituted with one or more R⁴.

In yet another embodiment, A is CH; and n is 1.

In one embodiment, A is N; and n is 1 or 2.

In another embodiment, A is COH; and n is 1.

In one embodiment, R² is C₃-C₆ cycloalkyl; and R³ is H or C₁-C₄ alkyl.

In one, embodiment, R² is C₁-C₇ alkoxy, NH(C₁-C₄ alkyl), or (CH₂)ᵥC(O)C₁-C₄ alkyl; R³ is H or C₁-C₄ alkyl; and v is 1 or 2.

In one embodiment, R² is C₁-C₄ alkyl; and R³ is H or C₁-C₄ alkyl.

In a separate embodiment, R² combines with R³ to form C₂-C₄ alkylene.

In a separate embodiment, R² combines with R³ to form methylene.

In one embodiment, R² is phenyl optionally substituted with one or more R⁵.

In another embodiment, R² is pyridyl, pyrimidyl, pyrazinyl or triazinyl, where the pyridyl, pyrimidyl, pyrazinyl and triazinyl is optionally substituted with one or more R⁵.

In one embodiment, R² is pyridyl optionally substituted with one or more R⁵.

In one embodiment, R² is pyrimidyl optionally substituted with one or more R⁵.

In another embodiment, R² is pyrazinyl optionally substituted with one or more R⁵.

In one embodiment, R² is triazinyl optionally substituted with one or more R⁵.

In one embodiment, R² is tetrazolyl, thiazoyl, oxazoyl, imidazoyl, isoxazoyl and pyrazoyl, where the tetrazolyl, thiazoyl, oxazoyl, imidazoyl, isoxazoyl and pyrazoyl are optionally substituted with one or more R⁵.

In one embodiment, R² is tetrazolyl optionally substituted with one or more R⁵.

In one embodiment, R² is thiazoyl optionally substituted with one or more R⁵.

In another embodiment, R² is oxazoyl optionally substituted with one or more R⁵.

In one embodiment, R² is imidazoyl optionally substituted with one or more R⁵.

In a separate embodiment, R² is isoxazoyl optionally substituted with one or more R⁵.

In one embodiment, R² is pyrazoyl optionally substituted with one or more R⁵.

In one embodiment, each R^{a} and R^{b} is independently H or C₁-C₄ alkyl; and wherein m is 0 or 1.

In another embodiment, R^{a} and R^{b} combine to form C₃-C₇ cycloalkyl; and wherein m is 0 or 1.

In one embodiment, each R⁴ is independently H or C₁-C₄ alkyl; and wherein m is 0 or 1.

In one embodiment, R⁴ is phenyl optionally substituted with one or more C₁-C₄ alkyl, C₁-C₄ alkoxy, fluorine or chlorine.

In a separate embodiment, R⁴ is C₁-C₄ alkyl or C₁-C₄ perfluoroalkyl.

In one embodiment, R⁵ is C₁-C₄ alkyl, fluorine or chlorine.

### Pharmaceutically Acceptable Salts

The present invention also comprises salts of the present compounds, typically, pharmaceutically acceptable salts. Such salts include pharmaceutically acceptable acid addition salts. Acid addition salts include salts of inorganic acids as well as organic acids.

Representative examples of suitable inorganic acids include hydrochloric, hydrobromic, hydroiodic, phosphoric, sulfuric, sulfamic, nitric acids and the like. Representative examples of suitable organic acids include formic, acetic, trichloroacetic, trifluoroacetic, propionic, benzoic, cinnamic, citric, fumaric, glycolic, itaconic, lactic, methanesulfonic, maleic, malic, malonic, mandelic, oxalic, picric, pyruvic, salicylic, succinic, methane sulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, theophylline acetic acids, as well as the 8-halotheophyllines (for example, 8-bromotheophylline and the like). Further examples of pharmaceutically acceptable inorganic or organic acid addition salts include the pharmaceutically acceptable salts listed in S. M. Berge, et al., J. Pharm. Sci., 1977, 66, 2.

Furthermore, the compounds of this invention may exist in unsolvated as well as in solvated forms with pharmaceutically acceptable solvents such as water, ethanol and the like.

Racemic forms may be resolved into the optical antipodes by known methods, for example, by separation of diastereomeric salts thereof with an optically active acid, and liberating the optically active amine compound by treatment with a base. Separation of such diastereomeric salts can be achieved, e.g. by fractional crystallization. The optically active acids suitable for this purpose may include, but are not limited to d- or l-tartaric, madelic or camphorsulfonic acids. Another method for resolving racemates into the optical antipodes is based upon chromatography on an optically active matrix. The compounds of the present invention may also be resolved by the formation and chromatographic separation of diastereomeric derivatives from chiral derivatizing reagents, such as, chiral alkylating or acylating reagents, followed by cleavage of the chiral auxiliary. Any of the above methods may be applied either to resolve the optical antipodes of the compounds of the invention per se or to resolve the optical antipodes of synthetic intermediates, which can then be converted by methods described herein into the optically resolved final products which are the compounds of the invention.

Additional methods for the resolution of optical isomers, known to those skilled in the art, may be used. Such methods include those discussed by J. Jaques, A. Collet and S. Wilen in Enantiomers, Racemates, and Resolutions, John Wiley and Sons, New York, 1981. Optically active compounds can also be prepared from optically active starting materials.

### Pharmaceutical Compositions

The present invention further provides a pharmaceutical composition comprising a therapeutically effective amount of a compound of Formula I and a pharmaceutically acceptable carrier. The present invention also provides a pharmaceutical composition comprising a therapeutically effective amount of one of the specific compounds disclosed in the Experimental Section and a pharmaceutically acceptable carrier.

The compounds of the invention may be administered alone or in combination with pharmaceutically acceptable carriers or excipients, in either single or multiple doses. The pharmaceutical compositions according to the invention may be formulated with pharmaceutically acceptable carriers or diluents as well as any other known adjuvants and excipients in accordance with conventional techniques such as those disclosed in Remington: The Science and Practice of Pharmacy, 19^{th} Edition, Gennaro, Ed., Mack Publishing Co., Easton, Pa., 1995.

The pharmaceutical compositions may be specifically formulated for administration by any suitable route such as oral, rectal, nasal, pulmonary, topical (including buccal and sublingual), transdermal, intracisternal, intraperitoneal, vaginal and parenteral (including subcutaneous, intramuscular, intrathecal, intravenous and intradermal) routes. It will be appreciated that the route will depend on the general condition and age of the subject to be treated, the nature of the condition to be treated and the active ingredient.

Pharmaceutical compositions for oral administration include solid dosage forms such as capsules, tablets, dragees, pills, lozenges, powders and granules. Where appropriate, the compositions may be prepared with coatings such as enteric coatings or they may be formulated so as to provide controlled release of the active ingredient such as sustained or prolonged release according to methods well known in the art. Liquid dosage forms for oral administration include solutions, emulsions, suspensions, syrups and elixirs.

Pharmaceutical compositions for parenteral administration include sterile aqueous and nonaqueous injectable solutions, dispersions, suspensions or emulsions as well as sterile powders to be reconstituted in sterile injectable solutions or dispersions prior to use.

Other suitable administration forms include, but are not limited to, suppositories, sprays, ointments, creams, gels, inhalants, dermal patches and implants.

Typical oral dosages range from about 0.001 to about 100 mg/kg body weight per day. Typical oral dosages also range from about 0.01 to about 50 mg/kg body weight per day. Typical oral dosages further range from about 0.05 to about 10 mg/kg body weight per day. Oral dosages are usually administered in one or more dosages, typically, one to three dosages per day. The exact dosage will depend upon the frequency and mode of administration, the sex, age, weight and general condition of the subject treated, the nature and severity of the condition treated and any concomitant diseases to be treated and other factors evident to those skilled in the art.

The formulations may also be presented in a unit dosage form by methods known to those skilled in the art. For illustrative purposes, a typical unit dosage form for oral administration may contain from about 0.01 to about 1000 mg, from about 0.05 to about 500 mg, or from about 0.5 to about 200 mg.

For parenteral routes such as intravenous, intrathecal, intramuscular and similar administration, typical doses are in the order of half the dose employed for oral administration.

The present invention also provides a process for making a pharmaceutical composition comprising admixing a therapeutically effective amount of a compound of Formula I and a pharmaceutically acceptable carrier. In an embodiment of the present invention the compound utilized in the aforementioned process is one of the specific compounds disclosed in the Experimental Section.

The compounds of this invention are generally utilized as the free substance or as a pharmaceutically acceptable salt thereof. One example is an acid addition salt of a compound having the utility of a free base. When a compound of Formula I contains a free base such salts are prepared in a conventional manner by treating a solution or suspension of a free base of Formula I with a molar equivalent of a pharmaceutically acceptable acid. Representative examples of suitable organic and inorganic acids are described above.

For parenteral administration, solutions of the compounds of Formula I in sterile aqueous solution, aqueous propylene glycol, aqueous vitamin E or sesame or peanut oil may be employed. Such aqueous solutions should be suitably buffered if necessary and the liquid diluent first rendered isotonic with sufficient saline or glucose. The aqueous solutions are particularly suitable for intravenous, intramuscular, subcutaneous and intraperitoneal administration. The compounds of Formula I may be readily incorporated into known sterile aqueous media using standard techniques known to those skilled in the art.

Suitable pharmaceutical carriers include inert solid diluents or fillers, sterile aqueous solutions and various organic solvents. Examples of solid carriers include lactose, terra alba, sucrose, cyclodextrin, talc, gelatin, agar, pectin, acacia, magnesium stearate, stearic acid and lower alkyl ethers of cellulose. Examples of liquid carriers include, but are not limited to, syrup, peanut oil, olive oil, phospholipids, fatty acids, fatty acid amines, polyoxyethylene and water. Similarly, the carrier or diluent may include any sustained release material known in the art, such as glyceryl monostearate or glyceryl distearate, alone or mixed with a wax. The pharmaceutical compositions formed by combining the compounds of Formula I and a pharmaceutically acceptable carrier are then readily administered in a variety of dosage forms suitable for the disclosed routes of administration. The formulations may conveniently be presented in unit dosage form by methods known in the art of pharmacy.

Formulations of the present invention suitable for oral administration may be presented as discrete units such as capsules or tablets, each containing a predetermined amount of the active ingredient, and optionally a suitable excipient. Furthermore, the orally available formulations may be in the form of a powder or granules, a solution or suspension in an aqueous or non-aqueous liquid, or an oil-in-water or water-in-oil liquid emulsion.

If a solid carrier is used for oral administration, the preparation may be tabletted, placed in a hard gelatin capsule in powder or pellet form or it may be in the form of a troche or lozenge. The amount of solid carrier will vary widely but will range from about 25 mg to about 1 g per dosage unit. If a liquid carrier is used, the preparation may be in the form of a syrup, emulsion, soft gelatin capsule or sterile injectable liquid such as an aqueous or non-aqueous liquid suspension or solution.

### Treatment of Disorders

As mentioned above, the compounds of Formula I are ligands at the NPY Y5 receptor. The present invention provides a method of treating a subject suffering from a mood disorder which comprises administering to the subject a therapeutically effective amount of a compound of this invention. The present invention provides a method of treating a subject suffering from a cognitive disorder which comprises administering to the subject a therapeutically effective amount of a compound of this invention. This invention further provides a method of treating a subject suffering from obesity which comprises administering to the subject a therapeutically effective amount of a compound of this invention. In an embodiment of this invention, the subject is a human being.

Furthermore, the present invention is directed to the use of a compound of this invention for the manufacture of a medicament useful for treating depression. Additionally, the present invention is directed to the use of a compound of this invention for the manufacture of a medicament useful for treating anxiety. The present invention further provides for the use of a compound of a compound of this invention for the manufacture of a medicament useful for treating obesity.

The invention will be better understood from the Experimental Details which follow. However, one skilled in the art will readily appreciate that the specific methods and results discussed therein are merely illustrative of the invention as described more fully in the claims which follow thereafter. Furthermore, the variables depicted in Schemes 1-6 are consistent with the variables recited in the Summary of the Invention.

One embodiment relates to a method of treating a subject suffering from mood disorders comprising administering to the subject a therapeutically effective amount of a compound of Formula I. In separate embodiments, the mood disorder is major depressive disorder; minor depressive disorder; dysthymia; cyclothymia; bipolar depression; and depression NUD; or depressive obesity.

Another embodiment relates to a method of treating a subject suffering from anxiety disorders comprising administering to the subject a therapeutically effective amount of a compound of Formula I. In separate embodiments, the mood anxiety is panic disorder; agoraphobia; social phobia (aka social anxiety disorder); obsessive compulsive disorder; or generalized anxiety disorder.

Yet another embodiment relates to a method of treating a subject suffering from stress-related disorders comprising administering to the subject a therapeutically effective amount of a compound of Formula I. In separate embodiments, the stress-related disorder is acute stress disorder; adjustment disorder; post traumatic stress disorder; exhaustion depression; or stress following (e.g. surgery and fever conditions).

Another embodiment relates to a method of treating a subject suffering from sleep disorders comprising administering to the subject a therapeutically effective amount of a compound of Formula I. In separate embodiments, the sleep disorders includes primary insomnia or a disorder related to disturbances in circadian rhythms.

Yet another embodiment relates to a method of treating a subject suffering from cognitive impairment/dysfunction disorders comprising administering to the subject a therapeutically effective amount of a compound of Formula I. In separate embodiments, the cognitive impairment/dysfunction is cognitive impairment associated with schizophrenia; dementias; autism; ADHD; or Alzheimer's disease. Moreover, “dementias” is further divided into age preceding dementia or AIDS dementia.

Another embodiment relates to a method of treating a subject suffering from substance dependency/abuse disorders comprising administering to the subject a therapeutically effective amount of a compound of Formula I. In separate embodiments, the substance dependency/abuse disorder is alcohol; nicotine; or cocaine addiction.

One embodiment relates to a method of treating a subject suffering from metabolic disorders comprising administering to the subject a therapeutically effective amount of a compound of Formula I. In separate embodiments, the metabolic disorder is dyslipidemia; hyperlipidemia; insulin hyposensitivity; overweight/obesity; hyperglycemia; metabolic syndrome; or diabetes mellitus.

Another embodiment relates to a method of treating a subject suffering from chronic pain disorders comprising administering to the subject a therapeutically effective amount of a compound of Formula I. In separate embodiments, the chronic pain disorders is neuropathic pain; neuralgic pain; migraine; fibromyalgia; IBS; chronic fatigue syndrome; chronic tension type headache; chronic low back pain; myofascial pain or chronic osteoarthritis.

### Experimental Section

General Methods: Anhydrous solvents were purchased from the Aldrich Chemical Company and used as received. The NMR spectra were measured on a Bruker Avance 400 spectrometer and or 300 MHz (Varian) with CDCl₃, DMSO-d₆ or CD₃OD as the solvent. Chemical shifts (8) are expressed in ppm, coupling constants (J) are expressed in Hz, and splitting patterns are described as follows: s=singlet; d=doublet; t=triplet; q=quartet; sept=septet; br=broad; m=multiplet; dd=doublet of doublets; dt=doublet of triplets; td=triplet of doublets; dq=doublet of quartet.

Unless otherwise noted, mass spectra were obtained using electrospray ionization (ESMS, Micromass Platform II or Quattro Micro) or Waters ZQ mass spectrometry with Agilent 1100 HPLC system with an autosampler using DAD/UV and Waters ELSD detection system and Inertsil ODS-3 column. For LC-MS determination, two methods were used: Method A: C18 column, Neutral pH, 20% to 90% Acetonitrile/H₂O with 0.2% Ammonium formate; or Method B: C8 column, Neutral pH, 10% to 90% Acetonitrile/H₂O with 0.2% Ammonium formate.

In certain instances, the methods of preparing the compounds of the invention are described generally by referring to representative reagents such as bases or solvents. The particular reagent identified is representative but is not inclusive and does not limit the invention in any way.

It is noted that scheme 6 describes the use of selective protecting groups during the synthesis of the compounds of the invention. One skilled in the art would be able to select the appropriate protecting group for a particular reaction. Moreover, it may be necessary to incorporate protection and deprotection strategies for substituents such as amino, amido, carboxylic acid and hydroxyl groups in the synthetic methods described below to synthesize the compounds of Formula I. Methods for protection and deprotection of such groups are well known in the art, and may be found in T. Green, et al., Protective Groups in Organic Synthesis, 1991, 2^{nd} Edition, John Wiley & Sons, New York.

### Preparation of the Compounds of Formula I

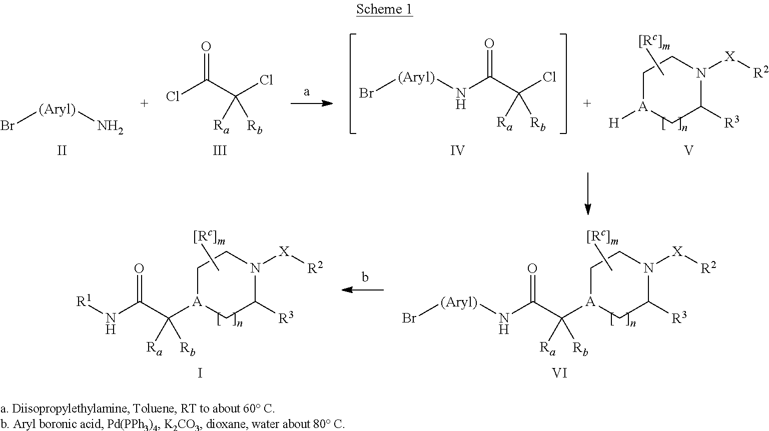

The compounds of Formula I, wherein A is N, may be synthesized according to the procedures described in Scheme 1. The starting materials of Formulas II and III are commercially available or may be synthesized by procedures known in the prior art. In summary, the aryl bromide of Formula II is coupled with the acid chloride of Formula III to afford the amide intermediate of Formula IV, which is generated in-situ or isolated to react with the amine of Formula V to provide the advanced intermediate of Formula VI. Coupling with aryl boronic acids with Formula VI affords the compounds of Formula I.

Representative Intermediates were Synthesized According to Scheme 1.

### Intermediate of Formula VI

2-(4-Acetyl-piperazin-1-yl)-N-(5-bromo-pyridin-2-yl)-acetamide: 2-Amino-5-bromopyridine (1.438 g, 8.312 mmol) and N,N-diisopropylethylamine (4.34 mL, 24.9 mmol) were dissolved in toluene (50 mL) at room temperature. Chloroacetyl chloride (0.73 mL, 9.14 mmol) was added and the reaction mixture was stirred for 2 hours at room temperature. To the reaction mixture was added 1-acetylpiperazine (2.13 g, 16.6 mmol). The reaction mixture was stirred at 60° C. for 2 hours. The reaction mixture was cooled to room temperature and transferred to a separatory funnel. The reaction mixture was washed with saturated aqueous sodium bicarbonate (2×50 mL) followed by water (1×50 mL). The organic phase was dried over magnesium sulfate, filtered, then concentrated in vacuo. The product was purified by flash chromatography eluting with 1/1 ethyl acetate/hexanes followed by 10/2/1 ethyl acetate/methanol/triethylamine to afford 1.56 g of the title compound. ¹H NMR (400 MHz, CDCl₃) δ 9.48 (br s, 1H), 8.34 (d, J=2.7 Hz, 1H), 8.18 (d, J=8.8 Hz, 1H), 7.82 (dd, J=8.8 Hz, J=2.7 Hz, 1H), 3.72 (t, J=5.0 Hz, 2H), 3.57 (t, J=5.2 Hz, 2H), 3.20 (s, 2H), 2.64-2.58 (m, 4H), 2.11 (s, 3H). ESI-MS m/z: 342.9 (M+H)⁺.

### Intermediate of Formula VI

N-(5-Bromo-pyridin-2-yl)-2-(6-oxo-hexahydro-pyrrolo[1,2-a]pyrazin-2-yl)-acetamide: N-(5-Bromo-2-pyridinyl)-2-chloroacetamide (253 mg, 1.01 mmol) was dissolved in DMF (5 mL) at room temperature. Hexahydro-pyrrolo[1,2-a]pyrazin-6-one (142 mg, 1.01 mmol) (Christensen et. al. WO2008046882) was added followed by potassium carbonate (350 mg, 2.53 mmol). The reaction mixture was stirred overnight at 60° C. The reaction mixture was cooled to room temperature, diluted with ethyl acetate (25 mL), and transferred to a separatory funnel. The reaction mixture was washed with water (3×25 mL). The organic phase was dried over magnesium sulfate, filtered through a pad of silica, then concentrated. The product was isolated by preparative thin layer chromatography eluting with 98/2 methylene chloride/methanol to afford 95 mg of the title compound. ¹H NMR (400 MHz, CDCl₃) δ 9.47 (br s, 1H), 8.34 (d, J=2.3 Hz, 1H), 8.18 (d, J=9.0 Hz, 1H), 7.82 (dd, J=9.0 Hz, J=2.3 Hz, 1H), 4.13-4.05 (m, 2H), 3.83-3.74 (m, 1H), 3.22 (dd, J=15.4 Hz, J=4.8 Hz, 2H), 3.07-2.96 (m, 2H), 2.91-2.86 (m, 2H), 2.48-2.39 (m, 2H), 2.33-2.17 (m, 2H), 1.68-1.57 (m, 1H). ESI-MS m/z: 354.9 (M+H)⁺.

### Compounds of the Invention

The following compounds were prepared according to the procedures in Scheme 1.

### Example 1a

### 2-(4-Acetyl-piperazin-1-yl)-N-[5-(3,5-difluoro-phenyl)-pyridin-2-yl]-acetamide

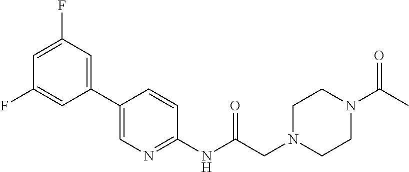

2-(4-Acetyl-piperazin-1-yl)-N-(5-bromo-pyridin-2-yl)-acetamide (171 mg, 0.50 mmol) was dissolved in 1,4-dioxane (4 mL) at room temperature. A solution of potassium carbonate in water (1M, 1 mL) was added followed by 3,5-difluorophenylboronic acid (119 mg, 0.75 mmol). Nitrogen was bubbled through the reaction mixture for 10 minutes. Tetrakis(triphenylphosphine)palladium(0) (116 mg, 0.1 mmol) was added. The reaction mixture was stirred under a nitrogen atmosphere at 80° C. for 3 hours. The reaction mixture was cooled to room temperature, diluted with 10 mL ethyl acetate and transferred to a separatory funnel. The organic phase was washed with aqueous sodium hydroxide solution (1N, 1×10 mL) followed by brine (1×10 mL). The organic phase was dried over magnesium sulfate, filtered, then concentrated. The product was purified by flash chromatography eluting with ethyl acetate followed by 9/1 ethyl acetate/methanol to give a brown oil. This was triturated from ether to afford 97 mg of the desired compound. ¹H NMR (400 MHz, CDCl₃) δ 9.70 (br s, 1H), 8.51 (d, J=2.1 Hz, 1H), 8.37 (d, J=8.8 Hz, 1H), 7.92 (dd, J=8.8 Hz, J=2.3 Hz, 1H), 7.09 (dt, J=6.6 Hz, J=2.1 Hz, 2H), 6.86 (dt, J=8.9 Hz, J=2.2 Hz, 1H), 3.78 (br s, 2H), 3.65 (br s, 2H), 2.70 (br s, 4H), 2.14 (s, 3H). ESI-MS m/z: 375.6 (M+H)⁺.

Likewise, the following compounds were prepared analogously to that of Example 1a:

### Example 1b

### 2-(4-Acetyl-piperazin-1-yl)-N-(5-phenyl-pyridin-2-yl)-acetamide

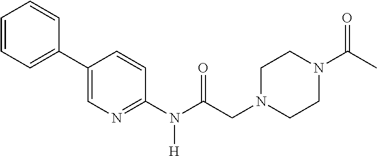

Prepared from 2-(4-acetyl-piperazin-1-yl)-N-(5-bromo-pyridin-2-yl)-acetamide and phenylboronic acid. LC-MS (m/z) 339.3 (MH⁺); t_{R}=1.05 min (Method A).

### Example 1c

### N-[5-(3,5-Difluoro-phenyl)-pyridin-2-yl]-2-(6-oxo-hexahydro-pyrrolo[1,2-a]pyrazin-2-yl)-acetamide

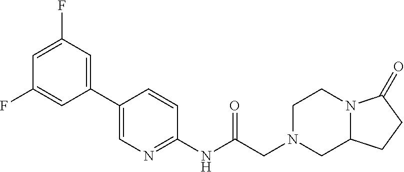

Prepared from N-(5-bromo-pyridin-2-yl)-2-(6-oxo-hexahydro-pyrrolo[1,2-a]pyrazin-2-yl)-acetamide and 3,5-difluorophenylboronic acid. ¹H NMR (400 MHz, CDCl₃) δ 9.55 (br s, 1H), 8.50 (dd, J=2.5 Hz, J=0.7 Hz, 1H), 8.34 (dd, J=8.6 Hz, J=0.7 Hz, 1H), 7.89 (dd, J=8.8 Hz, J=2.4 Hz, 1H), 7.10-7.06 (m, 2H), 6.84 (tt, J=8.8 Hz, J=1.6 Hz, 1H), 4.12-4.07 (m, 1H), 3.86-3.77 (m, 1H), 3.26 (dd, J=16.6 Hz, J=4.6 Hz, 2H), 3.10-3.00 (m, 2H), 2.92 (br d, J=11.2 Hz, 1H), 2.47-2.40 (m, 2H), 2.33 (dd, J=12.0 Hz, J=3.6 Hz, 1H), 2.28-2.16 (m, 1H), 1.69-1.56 (m, 1H). LC-MS (m/z) 387.0 (MH⁺); t_{R}=1.16 min (Method A).

### Example 1d

### 2-((R)-4-Acetyl-3-methyl-piperazin-1-yl)-N-[5-(3,5-difluoro-phenyl)-pyridin-2-yl]-acetamide

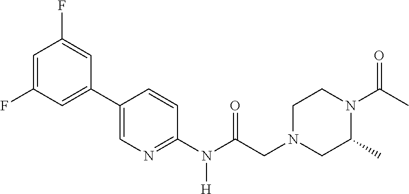

Prepared from 2-((R)-4-acetyl-3-methyl-piperazin-1-yl)-N-(5-bromo-pyridin-2-yl)-acetamide and 3,5-difluorophenylboronic acid. LC-MS (m/z) 389.0 (MH⁺); t_{R}=1.12 min (Method A).

### Example 1e

### 2-((S)-4-Acetyl-3-methyl-piperazin-1-yl)-N-[5-(3,5-difluoro-phenyl)-pyridin-2-yl]-acetamide

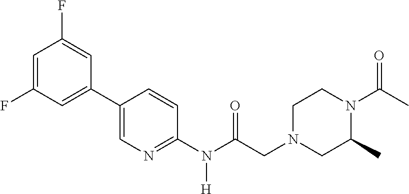

Prepared from 2-((S)-4-acetyl-3-methyl-piperazin-1-yl)-N-(5-bromo-pyridin-2-yl)-acetamide and 3,5-difluorophenylboronic acid. LC-MS (m/z) 389.0 (MH⁺); t_{R}=1.12 min (Method A).

### Example 1f

### 2-((R)-4-Acetyl-2-methyl-piperazin-1-yl)-N-[5-(3,5-difluoro-phenyl)-pyridin-2-yl]-acetamide

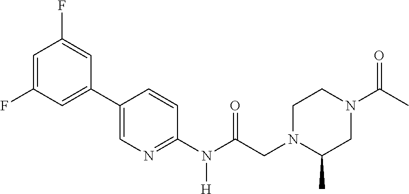

Prepared from 2-((R)-4-acetyl-2-methyl-piperazin-1-yl)-N-(5-bromo-pyridin-2-yl)-acetamide and 3,5-difluorophenylboronic acid. LC-MS (m/z) 389.1 (MH⁺); t_{R}=1.11 min (Method A).

### Example 1g

### 2-((S)-4-Acetyl-2-methyl-piperazin-1-yl)-N-[5-(3,5-difluoro-phenyl)-pyridin-2-yl]-acetamide

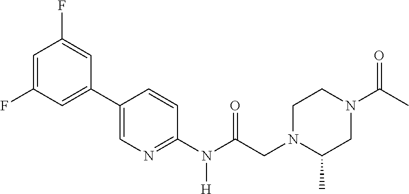

Prepared from 2-((S)-4-acetyl-2-methyl-piperazin-1-yl)-N-(5-bromo-pyridin-2-yl)-acetamide and 3,5-difluorophenylboronic acid. LC-MS (m/z) 389.1 (MH⁺); t_{R}=1.13 min (Method A).

### Example 1h

### 2-((R)-1-Acetyl-2-methyl-piperidin-4-yl)-N-[4-(3,5-difluoro-phenyl)-pyridin-2-yl]-acetamide

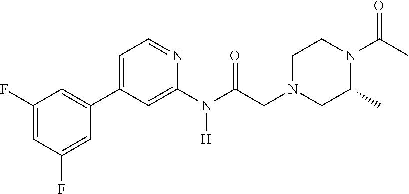

Prepared from 2-((R)-4-acetyl-3-methyl-piperazin-1-yl)-N-(4-bromo-pyridin-2-yl)-acetamide and 3,5-difluorophenylboronic acid. LC-MS (m/z) 389.0 (MH⁺); t_{R}=1.13 min (Method A).

### Example 1i

### N-[4-(3,5-Difluoro-phenyl)-pyridin-2-yl]-2-(6-oxo-hexahydro-pyrrolo[1,2-a]pyrazin-2-yl)-acetamide

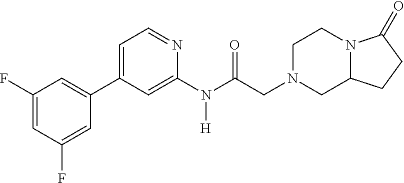

Prepared from N-(4-bromo-pyridin-2-yl)-2-(6-oxo-hexahydro-pyrrolo[1,2-a]pyrazin-2-yl)-acetamide and 3,5-difluorophenylboronic acid. LC-MS (m/z) 387.0 (MH⁺); t_{R}=1.02 min (Method A).

### Example 1j

### 2-(4-Acetyl-piperazin-1-yl)-N-[4-(3,5-difluoro-phenyl)-pyridin-2-yl]-acetamide

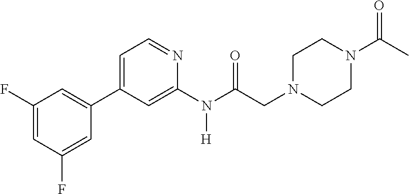

Prepared from 2-(4-acetyl-piperazin-1-yl)-N-(4-bromo-pyridin-2-yl)-acetamide and 3,5-difluorophenylboronic acid. LC-MS (m/z) 375.0 (MH⁺); t_{R}=1.01 min (Method A).

### Example 1k

### N-[6-(3,5-Difluoro-phenyl)-pyridin-2-yl]-2-(6-oxo-hexahydro-pyrrolo[1,2-a]pyrazin-2-yl)-acetamide

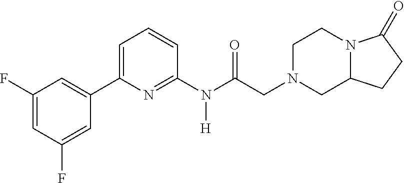

Prepared from N-(6-bromo-pyridin-2-yl)-2-(6-oxo-hexahydro-pyrrolo[1,2-a]pyrazin-2-yl)-acetamide and 3,5-difluorophenylboronic acid. LC-MS (m/z) 387.0 (MH⁺); t_{R}=1.10 min (Method A).

### Example 1l

### 2-(4-Acetyl-[1,4]diazepan-1-yl)-N-[5-(3,5-difluoro-phenyl)-pyridin-2-yl]-acetamide

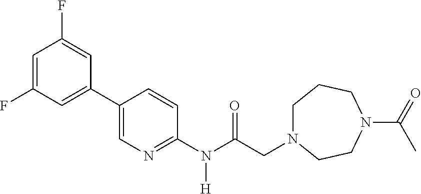

Prepared from 2-(4-acetyl-[1,4]diazepan-1-yl)-N-(5-bromo-pyridin-2-yl)-acetamide and 3,5-difluorophenylboronic acid. LC-MS (m/z) 389.0 (MH⁺); t_{R}=1.09 min (Method A).

### Example 1m

### N-[5-(3,5-Difluoro-phenyl)-pyridin-2-yl]-2-((S)-1,1-dioxo-hexahydro-1lambda*6*-thia-5,7a-diaza-inden-5-yl)-acetamide

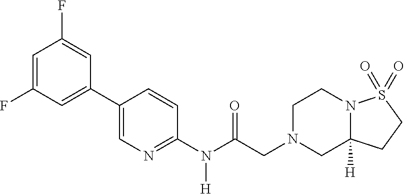

Prepared from N-(5-bromo-pyridin-2-yl)-2-((S)-1,1-dioxo-hexahydro-1lambda*6*-thia-5,7a-diaza-inden-5-yl)-acetamide and 3,5-difluorophenylboronic acid. LC-MS (m/z) 423.0 (MH⁺); t_{R}=1.13 min (Method A).

### Preparation of the Compounds of Formula I

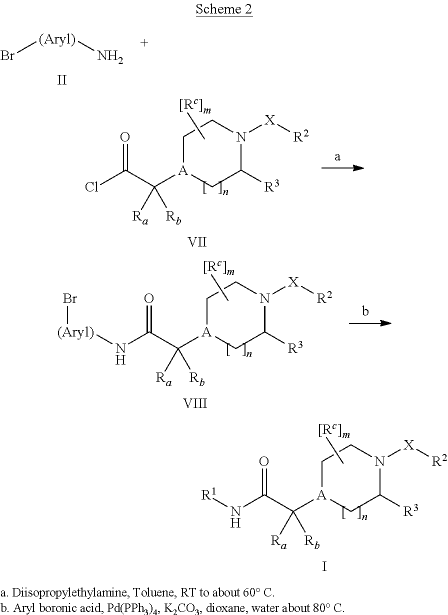

The compounds of Formula I may also be synthesized according to the procedures described in Scheme 2. The starting materials of Formulas II and VII are commercially available or may be synthesized by procedures known in the prior art. The aryl bromide of Formula II is coupled with the acid chloride of Formula VII to afford the amide intermediate of Formula VIII. Coupling with Aryl boronic acids affords the compounds of Formula I.

Representative Intermediates were Synthesized According to Scheme 2

### Intermediate of Formula VIII

2-(1-Acetyl-piperidin-4-yl)-N-(5-bromo-pyridin-2-yl)-acetamide: 2-Amino-5-bromopyridine (346 mg, 2.00 mmoles) and triethylamine (400 uL, 2.87 mmoles) were dissolved with stirring in tetrahydrofuran (15 ml). A solution of (1-acetyl-piperidin-4-yl)-acetyl chloride (407 mg, 2.00 mmoles) in dichloromethane (5 ml) was added and the reaction was allowed to stir overnight at room temperature. The reaction mixture was diluted with 100 ml ethyl acetate and washed with 50 ml water. The organic phase was dried over sodium sulfate, filtered and dried onto 2 g silica gel. The product was isolated by flash chromatography using 10% methanol in dichloromethane. The pure fractions were combined and dried to yield 276 mg of the title compound, 41% yield as a tan powder. NMR CDCl₃ δ 8.32 (d, 1H, J=2.4), 8.25 (bs, 1H), 8.17 (d, 1H, J=9.0), 7.81 (d, 1H, J=7.8), 4.65 (d, 1H, J=13.6), 3.82 (d, 1H, J=13.6), 3.1 (t, 1H, J=13.6), 2.59 (t, 1H, J=12.9), 2.37-2.32 (q, 2H), 2.17 (m, 1H), 2.10 (s, 3H), 1.84 (t, 2H, J=16.8), 1.31-1.12 (m, 2H). ESI-MS m/z: 342.9 (M+H)⁺.

### Compounds of the Invention

The compounds of Examples 2a-2an were prepared according to the procedures in Scheme 2.

### Example 2a

### 2-(1-Acetyl-piperidin-4-yl)-N-[5-(3,5-difluoro-phenyl)-pyridin-2-yl]-acetamide

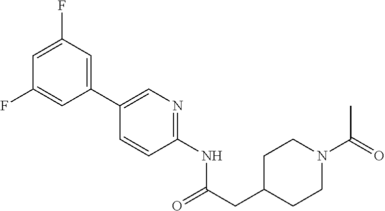

2-(1-Acetyl-piperidin-4-yl)-N-(5-bromo-pyridin-2-yl)-acetamide (300 mg, 0.818 mmole) was dissolved with stirring in 1,2-dimethoxyethane (15 ml) followed by the addition of 3,5-difluorophenylboronic acid (158 mg, 1.00 mmole). Next 1M aqueous potassium carbonate solution (5 ml, 5 mmole) was added and the reaction was degassed with nitrogen for 5 minutes. Tetrakis(triphenylphosphine)-palladium(0) (80 mg, 0.07 mmole) was added and the reaction mixture was heated at reflux under nitrogen for 2 hours. The reaction was diluted with ethyl acetate (100 mL) and washed with water (50 mls). The organic phase was dried over sodium sulfate, filtered and dried onto 2 g silica gel and purified by flash chromatography using 5% methanol in dichloromethane. The pure fractions were combined and triturated with ethyl acetate to yield 225 mg of white crystalline product 68.3% yield. NMR (CDCl₃) δ 8.46 (d, 1H, J=2.4), 8.33 (d, 1H, J=8.5), 7.94 (d, 1H, J=2.6), 7.91 (d, 1H, J=2.6), 7.1 (d, 2H, J=7.0), 6.64 (t, 1H, J=8.8), 4.6 (d, 1H, J=13.2), 3.84 (d, 1H, J=13.1), 3.11 (t, 1H, J=12.9), 2.61 (t, 1H, J=13.1), 2.4 (d, 2H, J=6.3), 2.2 (m, 1H), 2.1 (s, 3H), 1.86 (q, 2H, J=13.2), 1.25 (m, 2H). LC-MS (m/z) 374.1 (MH⁺); t_{R}=1.08 min (Method A).

Likewise, the following compounds were prepared analogously to that of Example 2a

### Example 2b

### 2-(1-Acetyl-piperidin-4-yl)-N-(2′-fluoro-biphenyl-4-yl)-acetamide

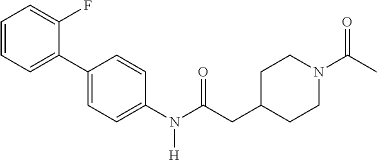

Prepared from 2-(1-acetyl-piperidin-4-yl)-N-(4-bromo-phenyl)-acetamide and 2-fluorophenyl boronic acid. LC-MS (m/z) 355.1 (MH⁺); t_{R}=1.10 min (Method A).

### Example 2c

### 2-(1-Acetyl-piperidin-4-yl)-N-(3′-fluoro-biphenyl-4-yl)-acetamide

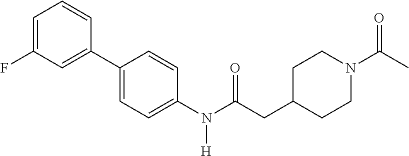

Prepared from 2-(1-acetyl-piperidin-4-yl)-N-(4-bromo-phenyl)-acetamide and 3-fluoro phenylboronic acid. LC-MS (m/z) 355.1 (MH⁺); t_{R}=1.12 min (Method A).

### Example 2d

### 2-(1-Acetyl-piperidin-4-yl)-N-(4′-fluoro-biphenyl-4-yl)-acetamide

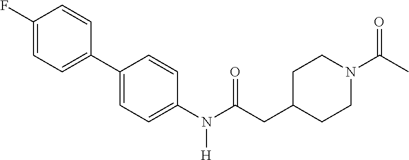

Prepared from 2-(1-acetyl-piperidin-4-yl)-N-(4-bromo-phenyl)-acetamide and 4-fluorophenyl boronic acid. LC-MS (m/z) 355.1 (MH⁺); t_{R}=1.10 min (Method A).

### Example 2e

### 2-(1-Acetyl-piperidin-4-yl)-N-(3′-methyl-biphenyl-4-yl)-acetamide

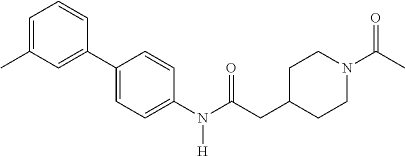

Prepared from 2-(1-acetyl-piperidin-4-yl)-N-(4-bromo-phenyl)-acetamide and 3-methylphenyl boronic acid. LC-MS (m/z) 351.3 (MH⁺); t_{R}=1.20 min (Method A).

### Example 2f

### 2-(1-Acetyl-piperidin-4-yl)-N-(4′-methyl-biphenyl-4-yl)-acetamide

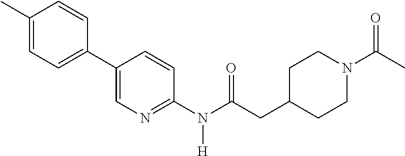

Prepared from 2-(1-acetyl-piperidin-4-yl)-N-(4-bromo-phenyl)-acetamide and 4-methylphenyl boronic acid. LC-MS (m/z) 351.1 (MH⁺); t_{R}=1.19 min (Method A).

### Example 2g

### 2-(1-Acetyl-piperidin-4-yl)-N-(2′-methoxy-biphenyl-4-yl)-acetamide

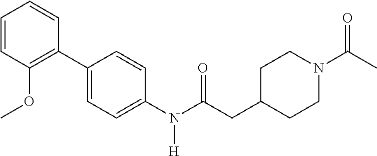

Prepared from 2-(1-acetyl-piperidin-4-yl)-N-(4-bromo-phenyl)-acetamide and 2-methoxyphenylboronic acid. LC-MS (m/z) 367.1 (MH⁺); t_{R}=1.07 min (Method A).

### Example 2h

### 2-(1-Acetyl-piperidin-4-yl)-N-(3′-methoxy-biphenyl-4-yl)-acetamide

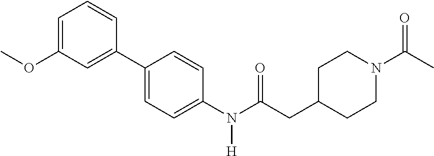

Prepared from 2-(1-acetyl-piperidin-4-yl)-N-(4-bromo-phenyl)-acetamide and 3-methoxyphenyl boronic acid. LC-MS (m/z) 367.1 (MH⁺); t_{R}=1.07 min (Method A).

### Example 2i

### 2-(1-Acetyl-piperidin-4-yl)-N-(4′-methoxy-biphenyl-4-yl)-acetamide

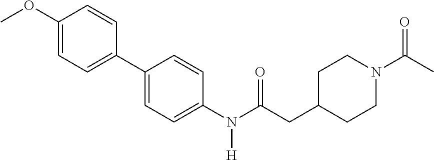

Prepared from 2-(1-acetyl-piperidin-4-yl)-N-(4-bromo-phenyl)-acetamide and 4-methoxyphenyl boronic acid. LC-MS (m/z) 367.1 (MH⁺); t_{R}=1.04 min (Method A).

### Example 2j

### 2-(1-Acetyl-piperidin-4-yl)-N-(2′,3′-difluoro-biphenyl-4-yl)-acetamide

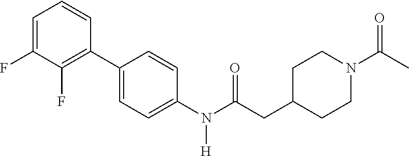

Prepared from 2-(1-acetyl-piperidin-4-yl)-N-(4-bromo-phenyl)-acetamide and 2,3-difluoro phenylboronic acid. LC-MS (m/z) 373.1 (MH⁺); t_{R}=1.15 min (Method A).

### Example 2k

### 2-(1-Acetyl-piperidin-4-yl)-N-(3′,4′-difluoro-biphenyl-4-yl)-acetamide

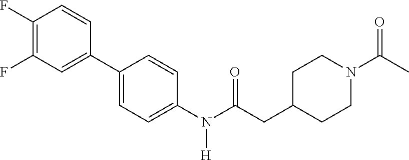

Prepared from 2-(1-acetyl-piperidin-4-yl)-N-(4-bromo-phenyl)-acetamide and 3,4-difluoro phenylboronic acid. LC-MS (m/z) 373.1 (MH⁺); t_{R}=1.17 min (Method A).

### Example 2l

### 2-(1-Acetyl-piperidin-4-yl)-N-(2′,5′-difluoro-biphenyl-4-yl)-acetamide

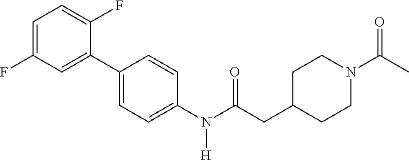

Prepared from 2-(1-acetyl-piperidin-4-yl)-N-(4-bromo-phenyl)-acetamide and 2,5-difluoro phenylboronic acid. LC-MS (m/z) 373.1 (MH⁺); t_{R}=1.14 min (Method A).

### Example 2m

### 2-(1-Acetyl-piperidin-4-yl)-N-(3′,5′-difluoro-biphenyl-4-yl)-acetamide

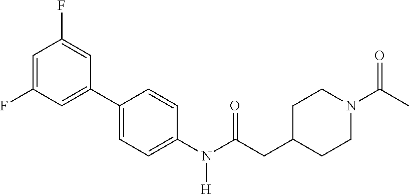

Prepared from 2-(1-acetyl-piperidin-4-yl)-N-(4-bromo-phenyl)-acetamide and 3,5-difluoro phenylboronic acid. LC-MS (m/z) 373.1 (MH⁺); t_{R}=1.19 min (Method A).

### Example 2n

### 2-(1-Acetyl-piperidin-4-yl)-N-(2′-methyl-biphenyl-4-yl)-acetamide

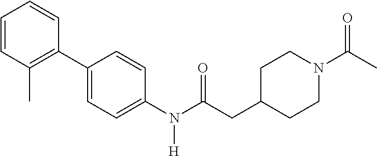

Prepared from 2-(1-acetyl-piperidin-4-yl)-N-(4-bromo-phenyl)-acetamide and 2-methylphenyl boronic acid. LC-MS (m/z) 351.1 (MH⁺); t_{R}=1.17 min (Method A).

### Example 2o

### 2-(1-Acetyl-4-methyl-piperidin-4-yl)-N-(3′,5′-difluoro-biphenyl-4-yl)-acetamide

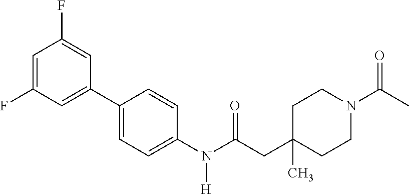

Prepared from 2-(1-acetyl-4-methyl-piperidin-4-yl)-N-(4-bromo-phenyl)-acetamide and 3,5-difluoro phenylboronic acid. LC-MS (m/z) 387.0 (MH⁺); t_{R}=1.29 min (Method A).

### Example 2p

### 2-(1-Acetyl-piperidin-4-yl)-N-[5-(3,5-difluoro-phenyl)-pyrimidin-2-yl]-acetamide

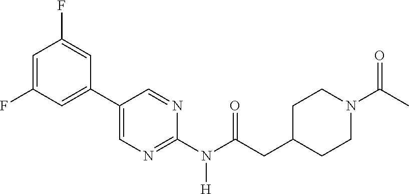

Prepared from 2-(1-acetyl-piperidin-4-yl)-N-(5-bromo-pyrimidin-2-yl)-acetamide and 3,5-difluorophenylboronic acid. LC-MS (m/z) 375.0 (MH⁺); t_{R}=0.79 min (Method A).

### Example 2q

### 2-(1-Acetyl-piperidin-4-yl)-N-[5-(3,5-dimethyl-phenyl)-pyrimidin-2-yl]-acetamide

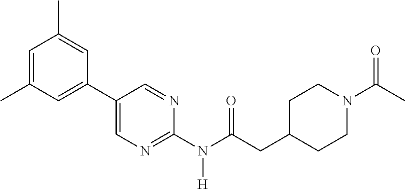

Prepared from 2-(1-acetyl-piperidin-4-yl)-N-(5-bromo-pyrimidin-2-yl)-acetamide and 3,5-dimethylphenylboronic acid. LC-MS (m/z) 367.0 (MH⁺); t_{R}=0.96 min (Method A).

### Example 2r

### 2-(1-Acetyl-piperidin-4-yl)-N-[6-(3,5-difluoro-phenyl)-pyridin-2-yl]-acetamide

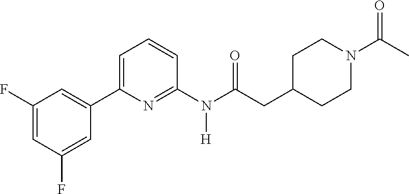

Prepared from 2-(1-acetyl-piperidin-4-yl)-N-(6-bromo-pyridin-2-yl)-acetamide and 3,5-difluoro phenylboronic acid. LC-MS (m/z) 374.0 (MH⁺); t_{R}=1.18 min (Method A).

### Example 2s

### 2-(1-Acetyl-piperidin-4-yl)-N-[5-(2-fluoro-phenyl)-pyridin-2-yl]-acetamide

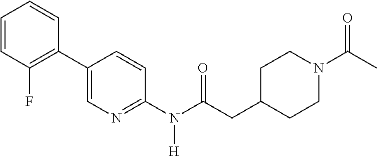

Prepared from 2-(1-acetyl-piperidin-4-yl)-N-(5-bromo-pyridin-2-yl)-acetamide and 2-fluoro phenylboronic acid. LC-MS (m/z) 356.1 (MH⁺); t_{R}=1.01 min (Method A).

### Example 2t

### 2-(1-Acetyl-piperidin-4-yl)-N-[5-(3,5-difluoro-phenyl)-pyrazin-2-yl]-acetamide

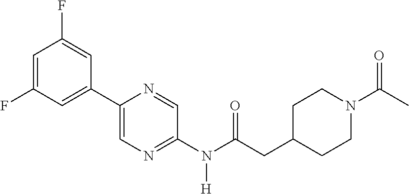

Prepared from 2-(1-acetyl-piperidin-4-yl)-N-(5-bromo-pyrazin-2-yl)-acetamide and 3,5-difluorophenylboronic acid. LC-MS (m/z) 375.1 (MH⁺); t_{R}=1.08 min (Method A).

### Example 2u

### 2-(1-Acetyl-piperidin-4-yl)-N-[5-(3,5-dichloro-phenyl)-pyrazin-2-yl]-acetamide

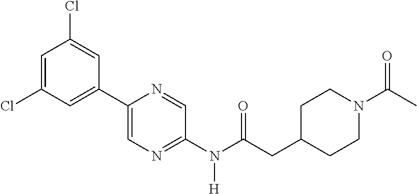

Prepared from 2-(1-acetyl-piperidin-4-yl)-N-(5-bromo-pyrazin-2-yl)-acetamide and 3,5-dichloro phenylboronic acid. LC-MS (m/z) 409.0 (MH⁺); t_{R}=1.08 min (Method A).

### Example 2v

### 2-(1-Acetyl-piperidin-4-yl)-N-[5-(3-chloro-phenyl)-pyrazin-2-yl]-acetamide

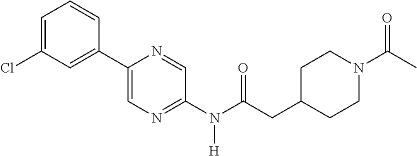

Prepared from 2-(1-acetyl-piperidin-4-yl)-N-(5-bromo-pyrazin-2-yl)-acetamide and 3-chloro phenylboronic acid. LC-MS (m/z) 374.1 (MH⁺); t_{R}=1.12 min (Method A).

### Example 2x

### 2-(1-Acetyl-piperidin-4-yl)-N-[5-(3-trifluoromethyl-phenyl)-pyridin-2-yl]-acetamide

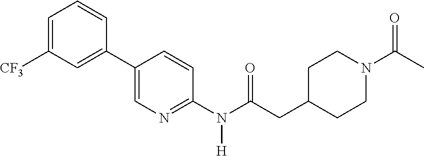

Prepared from 2-(1-acetyl-piperidin-4-yl)-N-(5-bromo-pyridin-2-yl)-acetamide and 3-trifluoromethylphenylboronic acid. LC-MS (m/z) 406.1 (MH⁺); t_{R}=1.21 min (Method A).

### Example 2y

### 2-(1-Acetyl-piperidin-4-yl)-N-[5-(3,5-difluoro-phenyl)-3-methyl-pyridin-2-yl]-acetamide

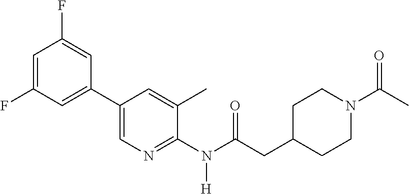

Prepared from 2-(1-acetyl-piperidin-4-yl)-N-(5-bromo-3-methyl-pyridin-2-yl)-acetamide and 3,5-difluorophenylboronic acid. LC-MS (m/z) 388.0 (MH⁺); t_{R}=0.96 min (Method A).

### Example 2z

### 2-(1-Acetyl-piperidin-4-yl)-N-[5-(3,5-difluoro-phenyl)-3,4-dimethyl-pyridin-2-yl]-acetamide

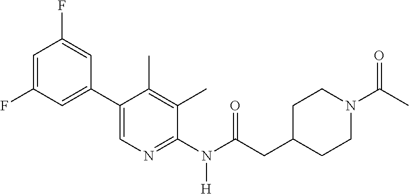

Prepared from 2-(1-acetyl-piperidin-4-yl)-N-(5-bromo-3,4-dimethyl-pyridin-2-yl)-acetamide and 3,5-difluorophenylboronic acid. LC-MS (m/z) 402.1 (MH⁺); t_{R}=0.98 min (Method A).

### Example 2aa

### 2-(1-Acetyl-piperidin-4-yl)-N-[5-(3,5-difluoro-phenyl)-6-methyl-pyridin-2-yl]-acetamide

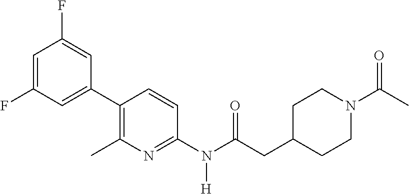

Prepared from 2-(1-acetyl-piperidin-4-yl)-N-(5-bromo-6-methyl-pyridin-2-yl)-acetamide and 3,5-difluorophenylboronic acid. LC-MS (m/z) 388.0 (MH⁺); t_{R}=1.17 min (Method A).

### Example 2ab

### 2-(1-Acetyl-piperidin-4-yl)-N-[5-(3,5-dichloro-phenyl)-6-methyl-pyridin-2-yl]-acetamide

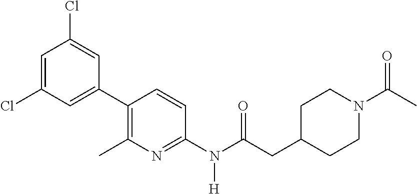

Prepared from 2-(1-acetyl-piperidin-4-yl)-N-(5-bromo-6-methyl-pyridin-2-yl)-acetamide and 3,5-dichlorophenylboronic acid. LC-MS (m/z) 421.1 (MH⁺); t_{R}=1.43 min (Method A).

### Example 2ac

### 2-(1-Acetyl-piperidin-4-yl)-N-[5-(2-fluoro-phenyl)-pyrazin-2-yl]-acetamide

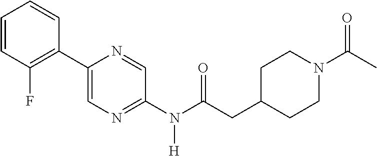

Prepared from 2-(1-acetyl-piperidin-4-yl)-N-(5-bromo-pyrazin-2-yl)-acetamide and 2-fluorophenylboronic acid. LC-MS (m/z) 357.0 (MH⁺); t_{R}=0.93 min (Method A).

### Example 2ad

### 2-(1-Acetyl-piperidin-4-yl)-N-[5-(2,4-difluoro-phenyl)-pyrazin-2-yl]-acetamide

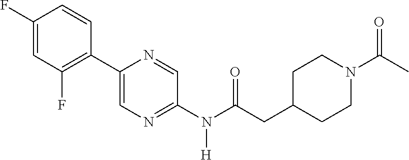

Prepared from 2-(1-acetyl-piperidin-4-yl)-N-(5-bromo-pyrazin-2-yl)-acetamide and 2,4-difluorophenylboronic acid. LC-MS (m/z) 375.0 (MH⁺); t_{R}=1.00 min (Method A).

### Example 2ae

### 2-(1-Acetyl-piperidin-4-yl)-N-(5-o-tolyl-pyridin-2-yl)-acetamide

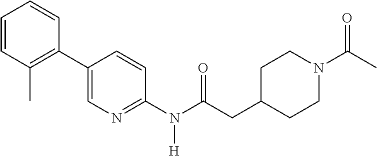

Prepared from 2-(1-acetyl-piperidin-4-yl)-N-(5-bromo-pyridin-2-yl)-acetamide and 2-methylphenylboronic acid. LC-MS (m/z) 352.0 (MH⁺); t_{R}=1.08 min (Method A).

### Example 2af

### 2-(1-Acetyl-piperidin-4-yl)-N-[5-(2,3,5-trifluoro-phenyl)-pyridin-2-yl]-acetamide

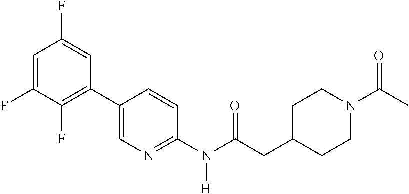

Prepared from 2-(1-acetyl-piperidin-4-yl)-N-(5-bromo-pyridin-2-yl)-acetamide and 2,3,5-trifluorophenylboronic acid. LC-MS (m/z) 392.0 (MH⁺); t_{R}=1.10 min (Method A).

### Example 2ag

### 2-(1-Acetyl-piperidin-4-yl)-N-[5-(2,3,5-trifluoro-phenyl)-pyrazin-2-yl]-acetamide

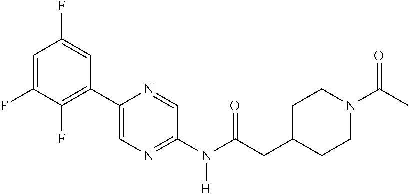

Prepared from 2-(1-acetyl-piperidin-4-yl)-N-(5-bromo-pyrazin-2-yl)-acetamide and 2,3,5-trifluorophenylboronic acid. LC-MS (m/z) 393.0 (MH⁺); t_{R}=1.09 min (Method A).

### Example 2ah

### 2-(1-Acetyl-piperidin-4-yl)-N-(4-phenyl-pyridin-2-yl)-acetamide

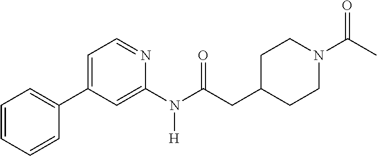

Prepared from 2-(1-acetyl-piperidin-4-yl)-N-(4-bromo-pyridin-2-yl)-acetamide and phenylboronic acid. LC-MS (m/z) 338.1 (MH⁺); t_{R}=0.95 min (Method A).

### Example 2ai

### 2-(1-Acetyl-piperidin-4-yl)-N-(4-p-tolyl-pyridin-2-yl)-acetamide

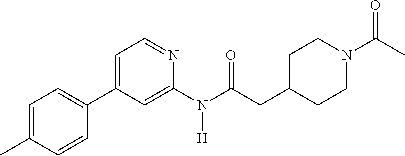

Prepared from 2-(1-acetyl-piperidin-4-yl)-N-(4-bromo-pyridin-2-yl)-acetamide and 4-methyl phenylboronic acid. LC-MS (m/z) 352.0 (MH⁺); t_{R}=1.09 min (Method A).

### Example 2aj

### N-[6-(3,5-Difluoro-phenyl)-pyridin-3-yl]-2-(1-methanesulfonyl-piperidin-4-yl)-acetamide

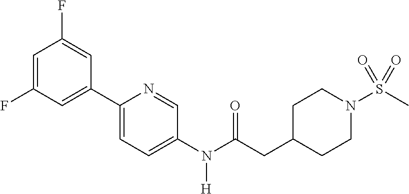

Prepared from N-(6-bromo-pyridin-3-yl)-2-(1-methanesulfonyl-piperidin-4-yl)-acetamide and 3,5-difluorophenylboronic acid. LC-MS (m/z) 410.1 (MH⁺); t_{R}=1.16 min (Method A).

### Example 2ak

### N-[6-(3,5-Difluoro-phenyl)-pyridin-3-yl]-2-[1-(propane-1-sulfonyl)-piperidin-4-yl]-acetamide

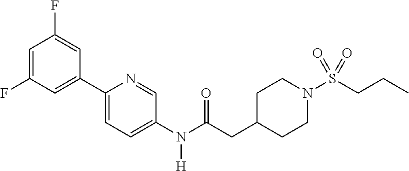

Prepared from N-(6-bromo-pyridin-3-yl)-2-[1-(propane-1-sulfonyl)-piperidin-4-yl]-acetamide and 3,5-difluorophenylboronic acid. LC-MS (m/z) 438.0 (MH⁺); t_{R}=1.35 min (Method A).

### Example 2al

### N-[5-(3,5-Difluoro-phenyl)-pyridin-2-yl]-2-(1-methanesulfonyl-piperidin-4-yl)-acetamide

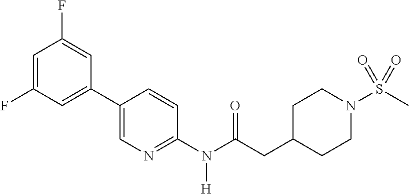

Prepared from N-(5-bromo-pyridin-2-yl)-2-(1-methanesulfonyl-piperidin-4-yl)-acetamide and 3,5-difluorophenylboronic acid. LC-MS (m/z) 410.0 (MH⁺); t_{R}=1.20 min (Method A).

### Example 2am

### N-[5-(3,5-Difluoro-phenyl)-pyridin-2-yl]-2-[1-(propane-1-sulfonyl)-piperidin-4-yl]-acetamide

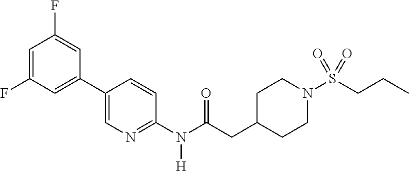

Prepared from N-(5-bromo-pyridin-2-yl)-2-[1-(propane-1-sulfonyl)-piperidin-4-yl]-acetamide and 3,5-difluorophenylboronic acid. LC-MS (m/z) 438.0 (MH⁺); t_{R}=1.40 min (Method A).

### Example 2an

### N-[5-(2-Fluoro-phenyl)-pyridin-2-yl]-2-(1-methanesulfonyl-piperidin-4-yl)-acetamide

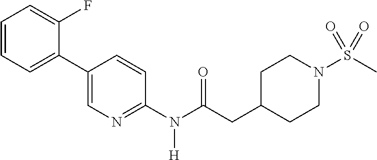

Prepared from N-(5-bromo-pyridin-2-yl)-2-(1-methanesulfonyl-piperidin-4-yl)-acetamide and 2-fluorophenylboronic acid. LC-MS (m/z) 392.1 (MH⁺); t_{R}=1.13 min (Method A).

### Preparation of the Compounds of Formula I

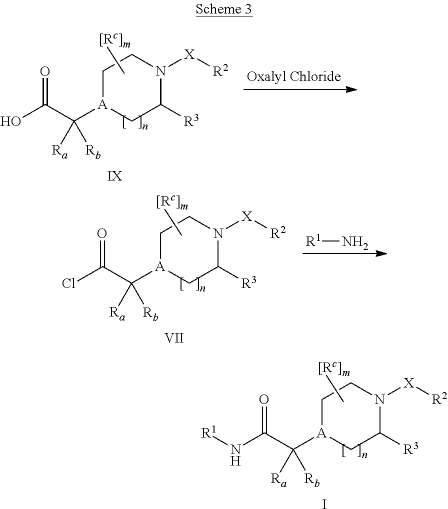

Alternatively, the compounds of Formula I may also be synthesized according to the procedures described in Scheme 3. The intermediate of Formula VII can be synthesized by reacting an acid of Formula IX with oxalyl chloride to form the acid chloride. The acid chloride is then coupled with R¹—NH₂ to afford the compounds of Formula I.

### Preparation of Advanced Intermediate of Formula IXa: (3-oxo-octahydro-indolizin-7-yl)-acetic acid

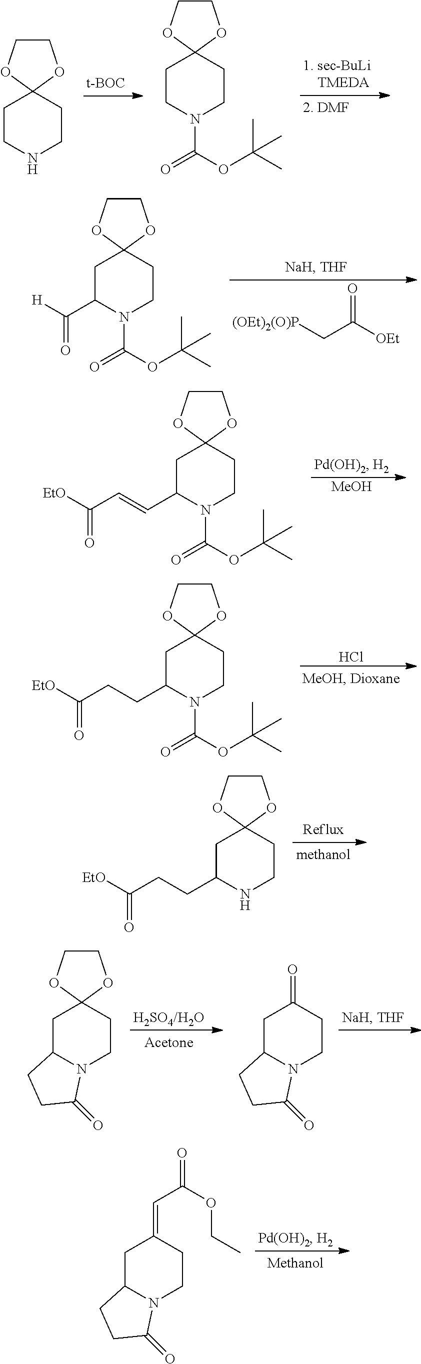

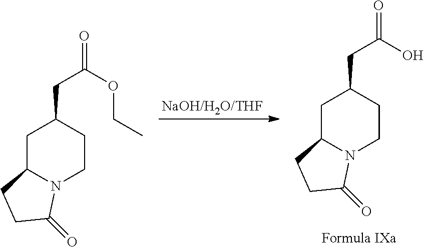

Step 1 1,4-dioxa-8-aza-spiro[4.5]decane-8-carboxylic acid tert-butyl ester: To a mixture of dioxane (200 mL) and an aqueous solution of NaOH (9.36 g, 234 mmol in 100 mL water) was added 1,4-dioxa-8-aza-spiro[4.5]decane. The mixture was cooled to 0° C. Di-tert-butyldicarbonate (37.4 g, 172 mmol) was added dropwise. The reaction mixture was allowed to stir at RT for 30 min, then was concentrated. The residue was suspended in 200 mL water and extracted with ethyl acetate. The combined organic layers were washed with brine, dried over sodium sulfate, and concentrated to provide the desired product as a white solid (35.4 g, 93%). 1H NMR CDCl₃, δ 4.00 (s, 4H), 3.53-3.47 (m, 4H), 1.70-1.65 (m, 4H), 1.44 (s, 9H); MS (ES, m/z) 244 M+H⁺.

Step 2 7-Formyl-1,4-dioxa-8-aza-spiro[4.5]decane-8-carboxylic acid tert-butyl ester: Into a round bottom flask was added 1,4-dioxa-8-aza-spiro[4.5]decane-8-carboxylic acid tert-butyl ester (3.54 g, 14.5 mmol), ether (40.0 mL) and TMEDA (2.03 g, 17.4 mmol). The reaction was cooled to −78° C. Sec-Butyllithium (12.5 mL, 1.4 M in cyclohexane) was added dropwise. The reaction was stirred at −78° C. for 2 hr, then was added DMF (2.13 g, 29.1 mmol). The mixture was stirred for 1 hr at −78° C., then was warmed to r.t. for 1 hr. The reaction was quenched by pouring into water. The aqueous solution was extracted with ethyl acetate. The combined organic phases were dried over anhydrous sodium sulfate then concentrated. The crude product was purified by column chromatography (hexane to ethyl acetate gradient) to provide the desired product as white solid (2.16, 55%). 1H NMR CDCl₃, δ9.58 (1H, S), 4.80-4.52 (m, 1H), 4.20-3.85 (m, 5H), 3.32-3.10 (m, 1H), 2.32-2.25 (m, 1H), 2.00-1.90 (m, 1H), 1.80-1.55 (m, 2H), 1.55-1.40 (b, 9H); MS (ES, m/z) 272 M+H⁺.

Step 3 7-((E)-2-ethoxycarbonyl-vinyl)-1,4-dioxa-8-aza-spiro[4.5]decane-8-carboxylic acid tert-butyl ester: To a suspension of NaH (446 mg, 11.1 mmol, 60% in mineral oil) in THF (30.0 mL) was added triethyl phosphonoacetate (2.50 g, 11.1 mmol) at room temperature. The reaction was stirred at r.t. for 15 min. Into the reaction at RT was added a solution of 7-formyl-1,4-dioxa-8-aza-spiro[4.5]decane-8-carboxylic acid tert-butyl ester (2.16 g, 7.96 mmol) in THF (10 mL). The reaction mixture was stirred at RT for 2 h, then was quenched by pouring into water. The aqueous solution was extracted with ethyl acetate. The combined organic phases were dried over anhydrous sodium sulfate then concentrated. The crude product was purified by column chromatography (hexane to ethyl acetate gradient) to give the desired product as a colorless oil (1.27, 47%). 1H NMR CDCl₃, δ7.10-7.00 (m, 1H), 5.82-5.77 (m, 1H), 5.10-5.00 (m, 1H), 4.25-4.18 (m, 2H), 4.12-4.05 (m, 1H), 4.00-3.93 (m, 4H), 3.15-3.08 (m, 1H), 2.05-1.95 (m, 1H), 1.92-1.85 (m, 1H), 1.70-1.60 (m, 2H), 1.50 (s, 9H), 1.35-1.25 (m, 3H); MS (ES, m/z) 242 M+H⁺.

Step 4 7-(2-Ethoxycarbonyl-ethyl)-1,4-dioxa-8-aza-spiro[4.5]decane-8-carboxylic acid tert-butyl ester: To a solution of 7-((E)-2-ethoxycarbonyl-vinyl)-1,4-dioxa-8-aza-spiro[4.5]decane-8-carboxylic acid tert-butyl ester (1.27 g, 3.72 mmol) in methanol (20.0 mL) was added Pd(OH)₂ (20% on charcoal, 0.261 g). The reaction mixture was purged with nitrogen then was stirred under a H₂ atmosphere (1 atm) overnight. The reaction mixture was filtered through celite and concentrated to give the desired product as pale yellow oil (1.28 g, 100%). 1H NMR CDCl₃, δ4.44-4.35 (m, 1H), 4.20-3.90 (m, 7H), 3.05-2.95 (m, 1H), 2.35-2.20 (m, 3H), 1.90-1.60 (m, 5H), 1.49 (s, 9H), 1.30-1.25 (m, 3H); MS (ES, m/z) 344 M+H⁺.

Step 5 3-(1,4-dioxa-8-aza-spiro[4.5]dec-7-yl)-propionic acid ethyl ester: To a solution of 7-(2-Ethoxycarbonyl-ethyl)-1,4-dioxa-8-aza-spiro[4.5]decane-8-carboxylic acid tert-butyl ester (1.28 g, 3.72 mmol) in methanol (20.0 mL) was added 4M HCl in dioxane solution (4.65 mL, 18.6 mmol). The mixture was allowed to stir at room temperature for 2 hr, then was concentrated. The resulting oil was dissolved in water and then basified with ammonium hydroxide aqueous solution, the aqueous solution was extracted with ethyl acetate, and the combined organic layers were dried, and concentrated. The crude material was used without further purification. MS (ES, m/z) 244 M+H⁺.

Step 6 Tetrahydro-1′H-spiro[[1,3]dioxolane-2,7′-indolizin]-3′(2′H)-one: A solution of 3-(1,4-dioxa-8-aza-spiro[4.5]dec-7-yl)-propionic acid ethyl ester generated in the previous step in methanol (20.0 mL) was refluxed overnight. The solution was concentrated and the residue was dissolved in ethyl acetate and filtered through a short plug of silica gel. The filtrate was concentrated to provide the crude product as pale yellow oil (733 mg, 100%). 1H NMR CD₃OD, δ4.76-4.65 (m, 5H), 4.45-4.35 (m, 1H), 3.55-3.45 (m, 1H), 3.33-3.28 (m, 1H), 3.05-2.88 (m, 3H), 2.69-2.62 (m, 1H), 2.48-2.44 (m, 1H), 2.38-2.15 (m, 3H); MS (ES, m/z) 198 M+H⁺.

Step 7 Hexahydro-indolizine-3,7-dione: To a solution of ketal protected bicyclic ketone (0.733 g, 3.72 mmol) in acetone (10.0 mL) was added water (10.0 mL) then sulfuric acid (0.500 mL, 9.40 mmol). The mixture was heated to 65° C. overnight. The reaction was cooled to r.t., then was concentrated. The residue was dissolved in water and was neutralized with ammonium hydroxide. The aqueous solution was extracted with ethyl acetate. The combined organic layers were dried, and concentrated to provide the desired product (569 mg, 100%). 1H NMR CDCl₃, δ4.29-4.22 (m, 1H), 3.78-3.70 (m, 1H), 2.92-2.84 (m, 1H), 2.52-2.45 (m, 1H), 2.38-2.15 (m, 6H), 1.68-1.58 (m, 1H); MS (ES, m/z) 154 M+H⁺.

Step 8 [3-oxo-hexahydro-indolizin-(7E)-ylidene]-acetic acid ethyl ester: To a suspension of sodium hydride (60% in mineral oil, 202 mg, 5.05 mmol) in THF (15 mL) was added triethyl phosphonoacetate (1.00 mL, 5.05 mmol). The mixture was allowed to stir at r.t. for 30 min, then was added hexahydro-indolizine-3,7-dione (645 mg, 4.21 mmol) in THF (5.00 mL). The reaction mixture was allowed to stir at r.t. for 3 hr, then was quenched by pouring to water. The aqueous solution was extracted with ethyl acetate. The combined organic layers were dried, and concentrated. The crude product was purified by column chromatography (hexane to ethyl acetate gradient) to provide the desired product (as a mixture of E/Z isomers) as a pale yellow oil (663 mg, 71%). 1H NMR CDCl₃, δ5.82-5.78 (s, 1H, two singlet), 4.25-3.85 (m, 4H), 3.58-3.48 (m, 1H), 2.80-2.65 (m, 1H), 2.50-2.20 (m, 4H), 2.14-2.90 (m, 1H), 1.80-1.60 (m, 1H), 1.35-1.25 (m, 3H); MS (ES, m/z) 224 M+H⁺.

Step 9 (3-oxo-octahydro-indolizin-7-yl)-acetic acid ethyl ester: To a solution of [3-oxo-hexahydro-indolizin-(7E)-ylidene]-acetic acid ethyl ester (663 mg, 2.97 mmol) in methanol (20.0 mL) was added Pd(OH)₂ (20% on charcoal, 208 mg). The reaction mixture was purged with nitrogen then was stirred under a H₂ atmosphere (1 atm) overnight. The reaction mixture was then filtered through celite and concentrated to give the desired product as a pale yellow oil. The crude material was used without further purification. MS (ES, m/z) 226 M+H⁺.

Step 10 (3-oxo-octahydro-indolizin-7-yl)-acetic acid: To a solution of (3-oxo-octahydro-indolizin-7-yl)-acetic acid ethyl ester (2.97 mmol, crude from previous step) in THF (20 mL) was added 10% NaOH aqueous solution (20 mL). The reaction mixture was stirred at r.t. overnight. The volatiles were removed, then the aqueous solution was acidified by dropwise addition of concentrated HCl. The aqueous solution was extracted with ethyl acetate. The combined organic layers were dried over magnesium sulfate, filtered and concentrated to provide the desired product (452 mg, 77%). 1H NMR CDCl₃, δ10.8-11.4 (br, 1H), 4.22-4.34 (m, 1H), 3.58-3.48 (m, 1H), 2.77-2.65 (m, 1H), 2.48-2.18 (m, 5H), 2.08-1.96 (m, 2H), 1.85-1.78 (m, 1H), 1.68-1.56 (m, 1H), 1.22-1.06 (m, 1H), 1.04-0.90 (m, 1H); MS (ES, m/z) 198 M+H⁺.

### Preparation of Advanced Intermediate of Formula IXb: ((7S,8aR)-3-oxo-hexahydro-oxazolo[3,4-a]pyridin-7-yl)-acetic acid

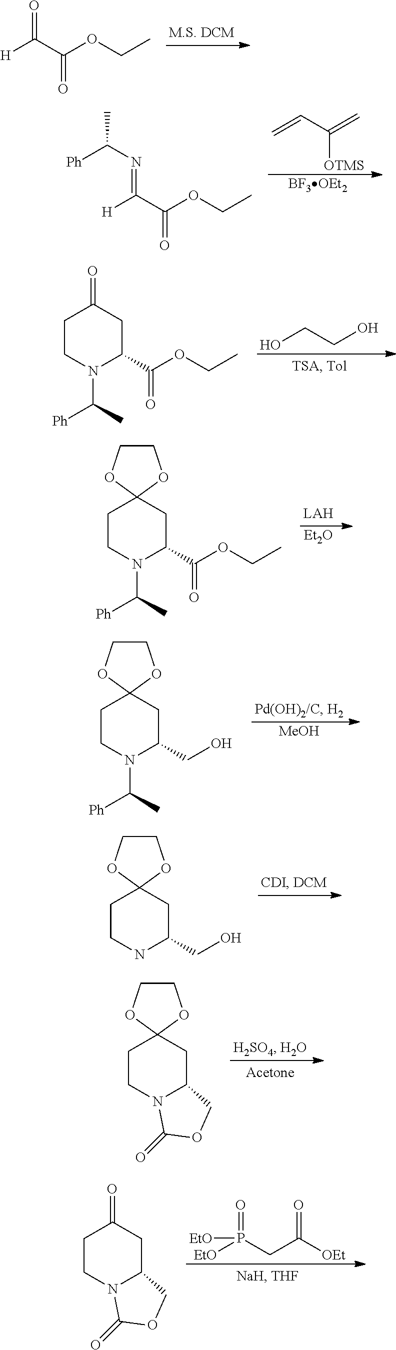

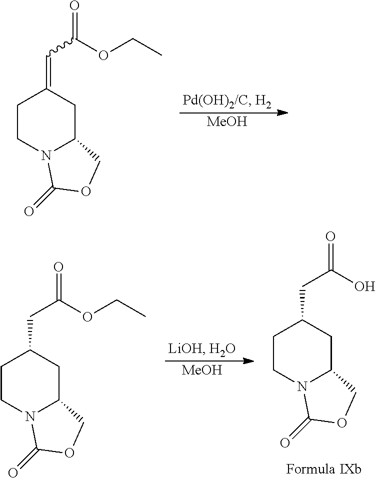

Step 1 ((S)-1-phenyl-ethylimino)-acetic acid ethyl ester: Into a round bottom flask was added ethyl glyoxylate (50% weight in toluene, 11.5 mL, 55.7 mmol). The solution was heated to reflux for 30 min, then cooled to room temperature. The above solution was added to (S)-α-methyl-benzylamine (7.18 mL, 55.7 mmol) in methylene chloride (100 mL) in the presence of molecular sieve (3 A, 6.50 g) at 0° C. The reaction mixture was allowed to warm to room temperature and was stirred at room temperature for one hour. The mixture was filtered, and the filtrate was concentrated and used directly for the next step without characterization.

Step 2 (R)-4-oxo-1-((S)-1-phenyl-ethyl)-piperidine-2-carboxylic acid ethyl ester: To a solution of ((S)-1-phenyl-ethylimino)-acetic acid ethyl ester (15.3 g, 55.7 mmol) in methylene chloride (100 mL) was added trifluoroacetic acid (6.34 g, 55.7 mmol) at −78° C. After stirring at −78° C. for 5 min, 2-trimethylsilyloxy-1,3-butadiene (7.91 g, 55.7 mmol) was added. The reaction mixture was stirred around −30° C. for 2 hr, then was quenched by adding water (20 mL). The mixture was stirred for 30 min at room temperature, and additional water (20 mL) was added. The pH value was adjusted to about 9 by adding solid NaHCO₃. The mixture was extracted with ethyl acetate. The organic phase was dried, filtered, and concentrated. The crude material was purified by column chromatography (hexane to 20% ethyl acetate in hexane gradient) to provide the desired product as a white solid (6.80 g, 44%). 1H NMR CDCl₃, δ 7.45-7.20 (m, 5H), 4.25-4.15 (m, 3H), 3.92-3.80 (m, 1H), 2.90-2.82 (m, 2H), 2.78-2.60 (m, 2H), 2.50-2.20 (m, 2H), 1.50-1.40 (m, 3H), 1.35-1.20 (m, 3H); MS (ES, m/z) 276 M+H⁺.

Step 3 (R)-8-((S)-1-phenyl-ethyl)-1,4-dioxa-8-aza-spiro[4.5]decane-7-carboxylic acid ethyl ester: To a round bottom flask fitted with a Dean-Stark trap was added (R)-4-oxo-1-((S)-1-phenyl-ethyl)-piperidine-2-carboxylic acid ethyl ester (6.80 g, 24.7 mmol), ethylene glycol (5.00 mL, 89.7 mmol), p-toluene sulfonic acid (425 mg, 2.47 mmol), and toluene (60.0 mL). The reaction was heated at reflux for 3 hr. The reaction was quenched by pouring into sat. NaHCO₃ aqueous solution, the aqueous solution was extracted with ethyl acetate. The combined organic phases were dried over anhydrous sodium sulfate and concentrated. The crude oil was purified by column chromatography (hexane to 20% ethyl acetate in hexane gradient) to afford the desired product as pale yellow solid (5.64 g, 71%). 1H NMR CDCl₃, δ 7.50-7.15 (m, 5H), 4.30-4.10 (m, 3H), 4.05-3.80 (m, 5H), 2.98-2.90 (m, 1H), 2.60-2.50 (m, 1H), 2.30-2.22 (m, 1H), 2.00-1.95 (m, 1H), 1.70-1.50 (m, 2H), 1.40-1.20 (m, 6H); MS (ES, m/z) 320 M+H⁺.

Step 4 [(R)-8-((S)-1-phenyl-ethyl)-1,4-dioxa-8-aza-spiro[4.5]dec-7-yl]-methanol: A solution of (R)-8-((S)-1-phenyl-ethyl)-1,4-dioxa-8-aza-spiro[4.5]decane-7-carboxylic acid ethyl ester (5.64 g, 17.6 mmol) in ether (10.0 mL) was added to a suspension of LAH (1.34 g, 35.3 mmol) in ether (100 mL) dropwise under nitrogen atmosphere. The reaction was allowed to stir overnight, and was quenched by adding water (1.5 mL), 20% NaOH solution (3.0 mL), and water (4.5 mL) in sequence. The mixture was stirred for 30 min, then was filtered through celite and washed with ethyl acetate. The filtrate was washed with brine, dried over anhydrous sodium sulfate, and concentrated. The resulting oil was used directly in the next step without further purification. 1H NMR CDCl₃, δ 7.50-7.25 (m, 5H), 4.30-4.15 (m, 1H), 4.05-3.90 (m, 4H), 3.75-3.60 (m, 2H), 3.00-2.90 (m, 1H), 2.80-2.70 (m, 2H), 2.55-2.45 (br, 1H), 1.90-1.80 (m, 1H), 1.75-1.60 (m, 2H), 1.55-1.45 (m, 1H), 1.40-1.30 (m, 3H); MS (ES, m/z) 278 M+H⁺.

Step 5 (R)-1-(1,4-dioxa-8-aza-spiro[4.5]dec-7-yl)-methanol: A mixture of [(R)-8-((S)-1-phenyl-ethyl)-1,4-dioxa-8-aza-spiro[4.5]dec-7-yl]-methanol (4.90 g, 17.7 mmol) and Pd(OH)₂ on charcoal in methanol (100 mL) was hydrogenated overnight at 50 psi on a Paar apparatus. The mixture was filtered through celite. The filtrate was concentrated and used in the next step without further purification. 1H NMR CD₃OD, δ 4.05-3.95 (m, 4H), 3.80-3.75 (m, 1H), 3.60-3.55 (m, 1H), 3.45-3.35 (m, 2H), 3.20-3.10 (m, 1H), 1.95-1.78 (m, 4H); MS (ES, m/z) 174 M+H⁺.

Step 6 (R)-7,7′-(1,3-dioxolane)-hexahydro-oxazolo[3,4-a]pyridin-3-one: N,N-carbonyldiimidazole (3.44 g, 3.70 mmol) was added to a solution of (R)-1-(1,4-dioxa-8-aza-spiro[4.5]dec-7-yl)-methanol (3.06 g, 17.7 mmol) in THF (30.0 mL). The reaction mixture was stirred at room temperature overnight. The reaction was quenched by pouring into water. The aqueous solution was extracted with ethyl acetate. The combined organic phases were dried over anhydrous sodium sulfate, filtered, and concentrated. The crude oil was purified by column chromatography (hexane to ethyl acetate gradient) to give the desired product as a pale yellow oil (2.67 g, 76%). 1H NMR CDCl₃, δ 4.50-4.40 (m, 1H), 4.05-3.90 (m, 7H), 3.15-3.05 (m, 1H), 1.95-1.85 (m, 1H), 1.75-1.60 (m, 3H); MS (ES, m/z) 200 M+H⁺.

Step 7 (R)-Tetrahydro-oxazolo[3,4-a]pyridine-3,7-dione: Sulfuric acid (3.52 mL, 66.0 mmol) was added dropwise to a solution of (R)-7,7′-(1,3-dioxolane)-hexahydro-oxazolo[3,4-a]pyridin-3-one (2.67 g, 13.5 mmol) in acetone (30.0 mL) and water (30.0 mL). The mixture was stirred at 70° C. overnight. The volatiles were removed under vacuum. The resulting aqueous solution was extracted with ethyl acetate. The combined organic phases were dried over anhydrous sodium sulfate, filtered and concentrated. The crude oil was purified by column chromatography (hexane to ethyl acetate gradient) to give the desired product as a white solid (1.15 g, 56%). 1H NMR CDCl₃, δ 4.55-4.47 (m, 1H), 4.30-4.22 (m, 1H), 4.10-4.05 (m, 2H), 3.30-3.18 (m, 1H), 2.63-2.40 (m, 4H).

Step 8 ((R)-3-Oxo-tetrahydro-oxazolo[3,4-a]pyridin-7-ylidene)-acetic acid ethyl ester: To a suspension of NaH (60% in mineral oil, 415 mg, 10.4 mmol) in THF (20 mL) was added triethyl phosphonoacetate (2.06 mL, 10.4 mmol). The mixture was stirred at room temperature for 30 min. A solution of (R)-tetrahydro-oxazolo[3,4-a]pyridine-3,7-dione in THF (10 mL) was added. The reaction mixture was stirred at room temperature for three hours, then was quenched by pouring into water. The aqueous solution was extracted with ethyl acetate. The combined organic phases were dried over anhydrous sodium sulfate, filtered and concentrated. The crude oil was purified by column chromatography (hexane to ethyl acetate gradient) to afford the desired product as a mixture of E/Z isomers (1.42 g, 85%). 1H NMR CDCl₃ for pure isomer one, δ 5.82 (s, 1H), 4.45-4.40 (m, 1H), 4.20-4.00 (m, 5H), 3.80-3.70 (m, 1H), 3.00-2.90 (m, 1H), 2.40-2.25 (m, 2H), 1.95-1.90 (m, 1H), 1.35-1.25 (m, 3H); MS (ES, m/z) 226 M+H⁺.

Step 9 ((7S,8aR)-3-Oxo-hexahydro-oxazolo[3,4-a]pyridin-7-yl)-acetic acid ethyl ester: A mixture of ((R)-3-oxo-tetrahydro-oxazolo[3,4-a]pyridin-7-ylidene)-acetic acid ethyl ester (1.42 g, 6.30 mmol) and Pd(OH)₂ (443 mg, 0.630 mmol) on charcoal in methanol (100 mL) was hydrogenated at balloon pressure overnight. The mixture was filtered through celite. The filtrate was concentrated and used directly in the next step without further purification. 1H NMR CDCl₃ δ 4.50-4.40 (m, 1H), 4.00-3.90 (m, 1H), 3.90-3.80 (m, 2H), 3.05-2.95 (m, 1H), 2.35-2.25 (m, 2H), 2.10-1.95 (m, 2H), 1.85-1.80 (m, 1H), 1.30-1.10 (m, 2H); MS (ES, m/z) 228 M+H⁺.

Step 10 ((7S,8aR)-3-Oxo-hexahydro-oxazolo[3,4-a]pyridin-7-yl)-acetic acid: To a solution of ((7S,8aR)-3-oxo-hexahydro-oxazolo[3,4-a]pyridin-7-yl)-acetic acid ethyl ester (1.43 g, 6.29 mmol) in methanol (30.0 mL) was added LiOH (753 mg, 31.5 mmol) and three drops of water. The mixture was stirred at room temperature overnight. The volatiles were removed under vacuum. The residual aqueous solution was acidified by adding concentrated HCl solution, then was extracted with ethyl acetate. The combined organic phases were dried over anhydrous sodium sulfate, filtered and concentrated. The crude solid was used in the next step without further purification (850 mg, 68%). 1H NMR CDCl₃ δ 4.40-4.30 (m, 1H), 4.15-4.00 (m, 2H), 3.85-3.75 (m, 2H), 3.75-3.65 (m, 1H), 2.90-2.80 (m, 1H), 2.25-2.15 (m, 2H), 2.00-1.85 (m, 2H), 1.70-1.60 (m, 1H), 1.25-1.00 (m, 5H); MS (ES, m/z) 200 M+H⁺.

### Preparation of Advanced Intermediate of Formula IXc: ((7S,8aR)-3-oxo-1,1′-dimethyl-hexahydro-oxazolo[3,4-a]pyridin-7-yl)-acetic acid

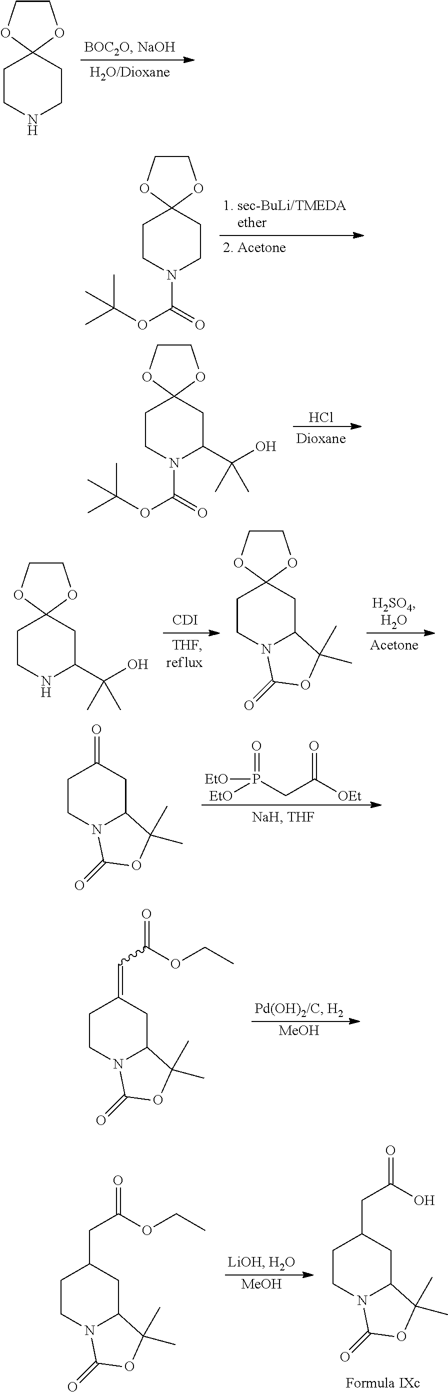

Step 1 1,4-dioxa-8-aza-spiro[4.5]decane-8-carboxylic acid tert-butyl ester: To a mixture of dioxane (200 mL) and an aqueous solution of NaOH (9.36 g, 234 mmol in 100 mL water) was added 1,4-dioxa-8-aza-spiro[4.5]decane. The mixture was cooled to 0° C. Di-tert-butyldicarbonate (37.4 g, 172 mmol) was added dropwise. The reaction mixture was allowed to stir at RT for 30 min, then was concentrated. The residue was suspended in 200 mL water and extracted with ethyl acetate. The combined organic phases were washed with brine, dried over sodium sulfate, filtered and concentrated to provide the desired product as a white solid (35.4 g, 93%). 1H NMR CDCl₃, δ 4.00 (s, 4H), 3.53-3.47 (m, 4H), 1.70-1.65 (m, 4H), 1.44 (s, 9H); MS (ES, m/z) 244 M+H⁺.

Step 2 7-(1-Hydroxy-1-methyl-ethyl)-1,4-dioxa-8-aza-spiro[4.5]decane-8-carboxylic acid tert-butyl ester: Into a round bottom flask was added 1,4-dioxa-8-aza-spiro[4.5]decane-8-carboxylic acid tert-butyl ester (5.038 g, 20.7 mmol) and ether (50.0 mL) and TMEDA (3.75 g, 24.8 mmol). The reaction was cooled to −78° C. To the reaction was added sec-BuLi (17.7 mL, 1.4 M in cyclohexane, 24.8 mmol) dropwise. The reaction was stirred at −78° C. for 2 hr, and then was treated with acetone (3.04 mL, 41.4 mmol). The mixture was stirred for 2 hr at −78° C. The reaction was quenched by pouring into the water, and the aqueous solution was extracted with ethyl acetate. The combined organic phases were dried over anhydrous sodium sulfate, filtered then concentrated. The crude product was purified by column chromatography (hexane to ethyl acetate gradient) to provide the desired product as a white solid (2.65 g, 42%). MS (ES, m/z) 302 M+H⁺.

Step 3 2-(1,4-Dioxa-8-aza-spiro[4.5]dec-7-yl)-propan-2-ol: To a solution of 7-(2-ethoxy carbonyl-ethyl)-1,4-dioxa-8-aza-spiro[4.5]decane-8-carboxylic acid tert-butyl ester (2.65 g, 8.79 mmol) in methanol (60.0 mL) was added 4M HCl in dioxane (11.0 mL, 44.0 mmol). The mixture was stirred at room temperature for 2 hr, and then was concentrated. The crude oil was dissolved in water and then basified with ammonium hydroxide aqueous solution. The aqueous solution was extracted with ethyl acetate. The combined organic layers were dried over sodium sulfate, filtered and concentrated. The crude product was used in the next step without further purification. MS (ES, m/z) 202 M+H⁺.

Step 4 (R)-7,7′-(1,3-Dioxolane)-1,1′-dimethyl-hexahydro-oxazolo[3,4-a]pyridin-3-one: N,N-Carbonyldiimidazole (2.14 g, 13.2 mmol) and DMAP (1.29 g, 10.6 mmol) were added to a solution of (R)-1-(1,4-dioxa-8-aza-spiro[4.5]dec-7-yl)-methanol (1.77 g, 8.79 mmol) in THF (60.0 mL). The reaction mixture was stirred at room temperature overnight. The reaction was quenched by pouring into water. The aqueous solution was extracted with ethyl acetate. The combined organic phases were dried over anhydrous sodium sulfate, filtered and concentrated. The crude oil was purified by column chromatography (ethyl acetate to 10% methanol in ethyl acetate gradient) to afford the desired product as a pale yellow oil (750 mg, 37%). 1H NMR CDCl₃, δ 4.10-3.90 (m, 4H), 3.57-3.50 (m, 1H), 3.25-3.20 (m, 1H), 3.12-3.05 (m, 1H), 1.73-1.55 (m, 4H), 1.47 (s, 3H), 1.33 (s, 3H); MS (ES, m/z) 228 M+H⁺.

Step 5 (R)-1,1′-Dimethyl-tetrahydro-oxazolo[3,4-a]pyridine-3,7-dione: Sulfuric acid (0.500 mL, 9.38 mmol) was added dropwise to a solution of (R)-7,7′-(1,3-dioxolane)-1,1′-dimethyl-hexahydro-oxazolo[3,4-a]pyridin-3-one (750 mg, 3.30 mmol) in acetone (34.3 mL) and water (34.3 mL). The mixture was stirred at 65° C. overnight. The volatiles were removed under vacuum. The resulting aqueous solution was extracted with ethyl acetate. The combined organic phases were dried over anhydrous sodium sulfate, filtered and concentrated. The crude oil was filtered through a short plug of silica gel and washed with ethyl acetate and methanol. The filtrate was concentrated and used directly in the next step (560 mg, 93%). 1H NMR CDCl₃, δ 4.25-4.15 (m, 1H), 3.55-3.50 (m, 1H), 3.15-3.05 (m, 1H), 2.55-2.30 (m, 4H), 1.42 (s, 3H), 1.31 (s, 3H); MS (ES, m/z) 184 M+H⁺.

Step 6 ((R)-3-Oxo-1,1′-dimethyl tetrahydro-oxazolo[3,4-a]pyridin-7-ylidene)-acetic acid ethyl ester: To a suspension of NaH (60% in mineral oil, 171 mg, 4.28 mmol) in THF (20 mL) was added triethyl phosphonoacetate (0.849 mL, 4.28 mmol). The mixture was stirred at room temperature for 30 min, then was added a solution of (R)-1,1′-dimethyl-tetrahydro-oxazolo[3,4-a]pyridine-3,7-dione (560 mg, 3.00 mmol) in THF (10 mL). The reaction mixture was stirred at room temperature for three hours. The reaction was quenched by pouring into water. The aqueous solution was extracted with ethyl acetate. The combined organic phases were dried over anhydrous sodium sulfate, filtered and concentrated. The crude oil was purified by column chromatography (hexane to ethyl acetate gradient) to give the desired product as a mixture of E/Z isomers (613 mg, 79%). 1H NMR CDCl₃ δ 5.82-5.70 (m, 1H, mix of E/Z), 4.20-3.80 (m, 4H), 3.35-3.20 (m, 1H), 2.93-2.75 (m, 1H), 2.30-1.75 (m, 3H), 1.50-1.15 (m, 9H); MS (ES, m/z) 254 M+H⁺.

Step 7 ((7S,8aR)-3-Oxo-1,1′-dimethyl-hexahydro-oxazolo[3,4-a]pyridin-7-yl)-acetic acid ethyl ester: A mixture of ((R)-3-oxo-1,1′-dimethyl-tetrahydro-oxazolo[3,4-a]pyridin-7-ylidene)-acetic acid ethyl ester (613 mg, 2.42 mmol) and 20% Pd(OH)₂ on charcoal (510 mg, 0.726 mmol) in methanol (40 mL) was hydrogenated at balloon pressure overnight. The mixture was filtered through celite. The filtrate was concentrated. The crude product was used in the next step without further purification. 1H NMR CDCl₃ δ 4.22-4.10 (m, 2H), 3.95-3.85 (m, 1H), 3.30-3.25 (m, 1H), 2.90-2.80 (m, 1H), 2.35-2.25 (m, 2H), 2.10-1.85 (m, 1H), 1.80-1.65 (m, 2H), 1.45 (s, 3H), 1.36-1.00 (m, 8H); MS (ES, m/z) 256 M+H⁺.

Step 8 ((7S,8aR)-3-Oxo-1,1′-dimethyl-hexahydro-oxazolo[3,4-a]pyridin-7-yl)-acetic acid: To a solution of ((7S,8aR)-3-oxo-1,1′-dimethyl-hexahydro-oxazolo[3,4-a]pyridin-7-yl)-acetic acid ethyl ester (614 mg, 2.40 mmol) in methanol (20.0 mL) was added LiOH (288 mg, 12.0 mmol) and a five drops of water. The mixture was stirred at room temperature overnight. The volatiles were removed under vacuum. The resulting aqueous solution was acidified by adding concentrated HCl solution, then was extracted with ethyl acetate. The combined organic phases were dried over anhydrous sodium sulfate, filtered and concentrated. The crude solid was used in the next step without further purification (546 mg, 99%). 1H NMR CDCl₃ δ 3.95-3.85 (m, 1H), 3.25-3.20 (m, 1H), 2.85-2.75 (m, 1H), 2.35-2.25 (m, 2H), 2.00-1.85 (m, 1H), 1.75-1.65 (m, 2H), 1.42 (s, 3H), 1.25 (s, 3H), 1.25-1.00 (m, 2H); MS (ES, m/z) 228 M+H⁺.

### Preparation of Advanced Intermediate

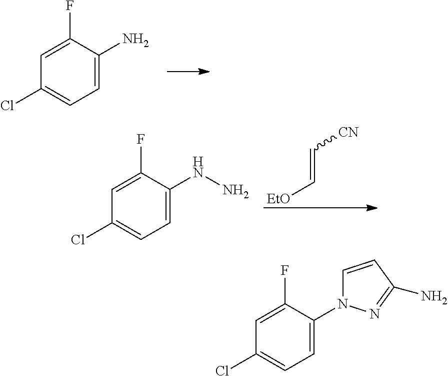

Step 1 (4-Chloro-2-fluorophenyl)-hydrazine: A flask was charged 4-chloro-2-fluoroanaline (5.09 g, 35.0 mmol), followed by dropwise addition of concentrated HCl solution (30.0 mL) at 0° C. The solution was stirred for 5 min at 0° C., then was added sodium nitrite (2.42 g, 35.1 mmol) in water (10.0 mL). The reaction was allowed to stir at room temperature for 30 min, then was cooled to 0° C. and was added tin (II) chloride dihydrate (15.9 g, 70.0 mmol) in a minimum amount of concentrated HCl solution. The reaction was stirred at 0° C. for 30 min, then room temperature for 4 hr. The reaction mixture was filtered and the solids were washed with cold ethanol. The solvent was removed in vacuo. The crude product was used directly in the next step without further purification (5.10 g, 62%). 1H NMR CD3OD δ 7.33-7.28 (m, 1H), 7.26-7.23 (m, 1H), 7.15-7.09 (m, 1H); MS (ES, m/z) 161 M+H⁺.

Step 2 1-(4-Chloro-2-fluoro-phenyl)-1H-pyrazol-3-ylamine: To a round bottom flask was charged (4-chloro-2-fluorophenyl)-hydrazine HCl salt (1.97 g, 10.0 mmol), ethanol (8.00 mL), 2.80 M sodium ethoxide in ethanol (10.0 mL, 28.0 mmol), and 3-ethoxyacrylonitrile (1.85 mL, 18.0 mmol). The reaction mixture was stirred at reflux overnight. The reaction mixture was cooled to room temperature and then was quenched by pouring into water. The aqueous solution was extracted with ethyl acetate. The combined organic phases were dried over anhydrous sodium sulfate, filtered and concentrated. The crude product was purified by column chromatography (hexane to 50% ethyl acetate in hexane gradient) to provide the desired product as a brown solid (334 mg, 16%). 1H NMR CD3OD 6 7.79 (br, 1H), 7.72-7.64 (m, 1H), 7.32-7.20 (m, 2H), 5.94 (br, 1H); MS (ES, m/z) 212 M+H⁺.

### Compounds of the Invention

The following compounds were prepared according to the procedures in Scheme 3.

### Example 3a

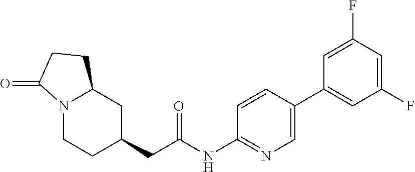

To a solution of (3-oxo-octahydro-indolizin-7-yl)-acetic acid (113 mg, 0.574 mmol) in dichloromethane (5.00 mL) was added oxalyl chloride (73.0 μL, 0.861 mmol), then was added DMF (7.00 μL, 0.0574 mmol). The reaction was allowed to stir at RT for 1 hr, then was concentrated and dried under high vacuum for 30 min. The crude acid chloride was redissolved in DCM (2.00 mL) and was added dropwise to the solution of 5-(3,5-difluoro-phenyl)-pyridin-2-ylamine (142 mg, 0.688 mmol) in DCM (3.00 mL) in the presence of triethyl amine (160 μL, 1.14 mmol). The reaction was allowed to stir for 1 hr at room temperature, and then was quenched by pouring into water. The aqueous solution was extracted with ethyl acetate, and the combined organic layers were dried and concentrated. The crude was purified by column chromatography (hexane to ethyl acetate gradient) to provide the desired product as white solid (20.8 mg, 9.4%). 1H NMR CDCl₃, δ8.60-8.40 (m, 2H), 8.28-8.36 (m, 1H), 7.92-7.86 (m, 1H), 7.12-7.02 (m, 2H), 6.88-6.78 (m, 1H), 4.20-4.10 (m, 1H), 3.58-3.45 (m, 1H), 2.78-2.44 (m, 1H), 2.50-2.00 (m, 7H), 1.90-1.50 (m, 2H), 1.25-1.10 (m, 1H), 1.10-0.90 (m, 1H); MS (ES, m/z) 386 MH⁺.

Likewise, the following compounds were prepared analogously to that of Example 3a.

### Example 3b

### 2-(1-Acetyl-piperidin-4-yl)-N-[6-(3,5-difluoro-phenyl)-pyridin-3-yl]-acetamide

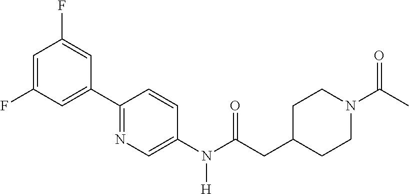

Prepared from (1-acetyl-piperidin-4-yl)-acetyl chloride and 6-(3,5-difluoro-phenyl)pyridin-3-ylamine. LC-MS (m/z) 374.0 (MH⁺); t_{R}=1.14 min (Method B).

### Example 3c

### 2-(1-Acetyl-piperidin-4-yl)-N-[6-(3-fluoro-phenyl)-pyridin-3-yl]-acetamide

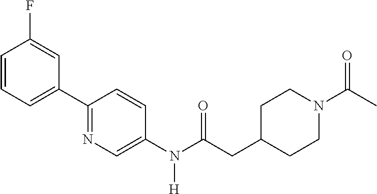

Prepared from (1-acetyl-piperidin-4-yl)-acetyl chloride and 6-(3-fluoro-phenyl)pyridin-3-ylamine. LC-MS (m/z) 356.0 (MH⁺); t_{R}=1.06 min (Method B).

### Example 3d

### 2-(1-Acetyl-piperidin-4-yl)-N-[6-(3-methoxy-phenyl)-pyridin-3-yl]-acetamide

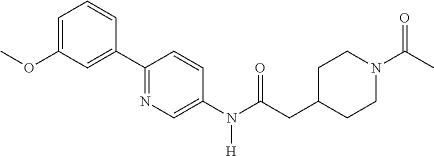

Prepared from (1-acetyl-piperidin-4-yl)-acetyl chloride and 6-(3-methoxy-phenyl)pyridin-3-ylamine. LC-MS (m/z) 368.0 (MH⁺); t_{R}=1.01 min (Method B).

### Example 3e

### 2-(1-Acetyl-piperidin-4-yl)-N-[5-(3,5-dichloro-phenyl)-pyridin-2-yl]-acetamide

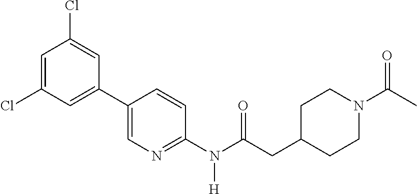

Prepared from (1-acetyl-piperidin-4-yl)-acetyl chloride and 5-(3,5-dichloro-phenyl)pyridin-2-ylamine. LC-MS (m/z) 407.1 (MH⁺); t_{R}=1.34 min (Method A).

### Example 3f

### 2-(1-Acetyl-piperidin-4-yl)-N-[4-(3,5-difluoro-phenyl)-pyridin-2-yl]-acetamide

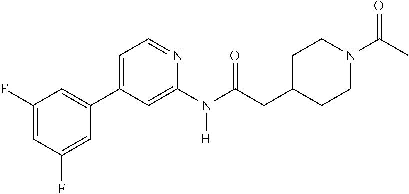

Prepared from (1-acetyl-piperidin-4-yl)-acetyl chloride and 4-(3,5-difluoro-phenyl)pyridin-2-ylamine. LC-MS (m/z) 374.0 (MH⁺); t_{R}=1.07 min (Method A).

### Example 3g

### N-[5-(3,5-Difluoro-phenyl)-pyridin-2-yl]-2-(1,1-dimethyl-3-oxo-hexahydro-oxazolo[3,4-a]pyridin-7-yl)-acetamide

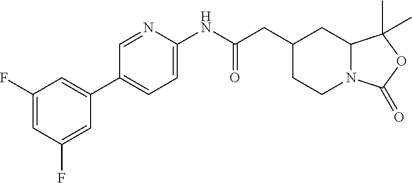

Prepared from 5-(3,5-difluoro-phenyl)-pyridin-2-ylamine and (1,1-dimethyl-3-oxo-hexahydro-oxazolo[3,4-a]pyridin-7-yl)-acetic acid. 1H NMR CDCl3 δ 8.50-8.46 (m, 1H), 8.35-8.30 (m, 1H), 8.14 (br, 1H), 7.94-7.90 (m, 1H), 7.12-7.08 (m, 2H), 6.89-6.83 (m, 1H), 4.00-3.92 (m, 1H), 3.36-3.30 (m, 1H), 2.97-2.90 (m, 1H), 2.50-2.36 (m, 2H), 2.24-2.10 (m, 1H), 1.86-1.78 (m, 2H), 1.46 (s, 3H), 1.33 (s, 3H), 1.33-1.10 (m, 2H); MS (ES, m/z) 416 M+H⁺ (Method B).

### Example 3h

### N-[5-(3,5-Difluoro-phenyl)-pyrazin-2-yl]-2-(1,1-dimethyl-3-oxo-hexahydro-oxazolo[3,4-a]pyridin-7-yl)-acetamide

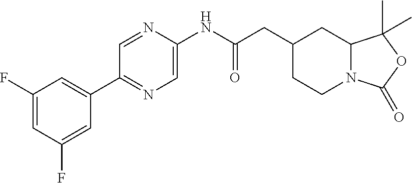

Prepared from 5-(3,5-difluoro-phenyl)-pyrazin-2-ylamine and (1,1-dimethyl-3-oxo-hexahydro-oxazolo[3,4-a]pyridin-7-yl)-acetic acid. 1H NMR CDCl3 δ 9.60 (br, 1H), 8.65 (m, 1H), 8.03 (br, 1H), 7.58-7.50 (m, 2H), 6.93-6.84 (m, 1H), 4.00-3.92 (m, 1H), 3.36-3.30 (m, 1H), 2.98-2.88 (m, 1H), 2.57-2.41 (m, 2H), 2.26-2.14 (m, 1H), 1.88-1.78 (m, 2H), 1.46 (s, 3H), 1.32 (s, 3H), 1.32-1.10 (m, 2H); MS (ES, m/z) 417 M+H⁺ (Method B).

### Example 3i

### 2-(1,1-Dimethyl-3-oxo-hexahydro-oxazolo[3,4-a]pyridin-7-yl)-N-[1-(2-fluoro-phenyl)-1H-pyrazol-3-yl]-acetamide

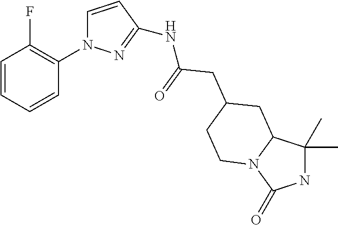

Prepared from 1-(2-fluoro-phenyl)-1H-pyrazol-3-yl-amine and (1,1-dimethyl-3-oxo-hexahydro-oxazolo[3,4-a]pyridin-7-yl)-acetic acid. 1H NMR CDCl3 δ 8.75 (br, 1H), 7.93-7.91 (m, 1H), 7.77-7.73 (m, 1H), 7.31-7.21 (m, 3H), 7.00-6.99 (m, 1H), 3.91-3.85 (m, 1H), 3.28-3.24 (m, 1H), 2.90-2.81 (m, 1H), 2.29-2.01 (m, 3H), 1.75-1.63 (m, 2H), 1.41 (s, 3H), 1.29 (s, 3H), 1.11-0.85 (m, 2H); MS (ES, m/z) 387 M+H⁺ (Method B).

### Example 3j

### N-[5-(3,5-Difluoro-phenyl)-pyridin-2-yl]-2-(3-oxo-hexahydro-oxazolo[3,4-a]pyridin-7-yl)-acetamide

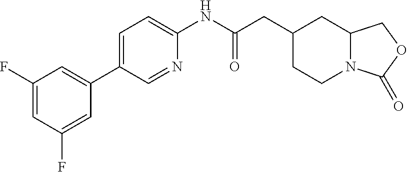

Prepared from 5-(3,5-difluoro-phenyl)-pyridin-2-ylamine and (3-oxo-hexahydro-oxazolo[3,4-a]pyridin-7-yl)-acetic acid. 1H NMR CDCl3 δ 8.47 (br, 1H), 8.36-8.28 (m, 2H), 7.94-7.89 (m, 1H), 7.10-7.04 (m, 2H), 6.88-6.80 (m, 1H), 4.45-4.40 (m, 1H), 3.98-3.88 (m, 2H), 3.82-3.72 (m, 1H), 3.00-2.90 (m, 1H), 2.50-2.34 (m, 2H), 2.30-2.12 (m, 1H), 2.04-1.96 (m, 1H), 1.86-1.79 (m, 1H), 1.34-1.14 (m, 2H); MS (ES, m/z) 388 M+H⁺ (Method B).

### Example 3k

### N-[5-(3-Fluoro-phenyl)-pyrazin-2-yl]-2-(3-oxo-hexahydro-oxazolo[3,4-a]pyridin-7-yl)-acetamide

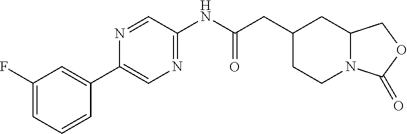

Prepared from 5-(3-fluoro-phenyl)-pyrazin-2-ylamine and (3-oxo-hexahydro-oxazolo[3,4-a]pyridin-7-yl)-acetic acid. 1H NMR CDCl3 δ 9.58-9.46 (br, 1H), 8.62-8.50 (br, 1H), 7.66 (br, 1H), 7.58-7.54 (m, 2H), 7.48-7.44 (m, 1H), 7.04-7.02 (m, 1H), 4.40-4.34 (m, 1H), 3.92-3.80 (m, 2H), 3.78-3.68 (m, 1H), 2.94-2.84 (m, 1H), 2.46-2.30 (m, 2H), 2.26-2.10 (m, 1H), 2.00-1.92 (m, 1H), 1.80-1.72 (m, 1H), 1.28-1.06 (m, 2H); MS (ES, m/z) 371 M+H⁺ (Method B).

### Example 3l

### N-[5-(3,5-Difluoro-phenyl)-pyrazin-2-yl]-2-(3-oxo-hexahydro-oxazolo[3,4-a]pyridin-7-yl)-acetamide

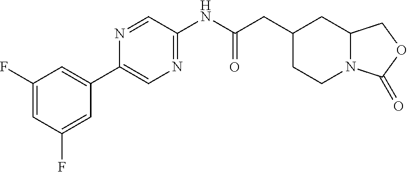

Prepared from 5-(3,5-difluoro-phenyl)-pyrazin-2-ylamine and (3-oxo-hexahydro-oxazolo[3,4-a]pyridin-7-yl)-acetic acid. 1H NMR CDCl3 δ 9.64-9.52 (br, 1H), 8.76-8.56 (br, 1H), 8.08-8.02 (br, 1H), 7.58-7.48 (m, 2H), 6.92-6.86 (m, 1H), 4.46-4.42 (m, 1H), 4.00-3.90 (m, 2H), 3.84-3.74 (m, 1H), 3.02-2.92 (m, 1H), 2.52-2.38 (m, 2H), 2.32-2.18 (m, 1H), 2.06-2.00 (m, 1H), 1.88-1.80 (m, 1H), 1.34-1.02 (m, 2H); MS (ES, m/z) 389 M+H⁺ (Method B).

### Example 3m

### N-[5-(3-Chloro-phenyl)-pyridin-2-yl]-2-(3-oxo-hexahydro-oxazolo[3,4-a]pyridin-7-yl)-acetamide

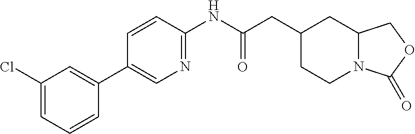

Prepared from 5-(3-chloro-phenyl)-pyridin-2-ylamine and (3-oxo-hexahydro-oxazolo[3,4-a]pyridin-7-yl)-acetic acid. 1H NMR CDCl3 δ 8.54-8.46 (br, 1H), 8.34-8.28 (m, 1H), 8.22-8.16 (br, 1H), 7.96-7.92 (m, 1H), 7.58-7.54 (m, 1H), 7.50-7.36 (m, 3H), 4.48-4.40 (m, 1H), 4.00-3.70 (m, 3H), 3.02-2.92 (m, 1H), 2.50-2.34 (m, 2H), 2.30-2.14 (m, 1H), 2.06-2.00 (m, 1H), 1.88-1.80 (m, 1H), 1.36-1.10 (m, 2H); MS (ES, m/z) 386 M+H⁺ (Method B).

### Example 3n

### N-[5-(3,5-Difluoro-phenyl)-pyrimidin-2-yl]-2-(3-oxo-hexahydro-oxazolo[3,4-a]pyridin-7-yl)-acetamide

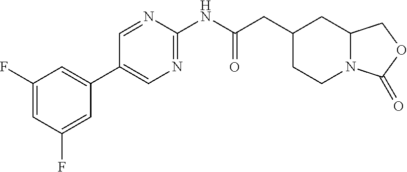

Prepared from 5-(3,5-difluoro-phenyl)-pyrimidin-2-ylamine and (3-oxo-hexahydro-oxazolo[3,4-a]pyridin-7-yl)-acetic acid. 1H NMR CDCl3 δ 8.96-8.90 (br, 1H), 8.86-8.76 (br, 2H), 7.12-7.06 (m, 2H), 6.96-6.86 (m, 1H), 4.48-4.40 (m, 1H), 4.00-3.72 (m, 3H), 3.02-2.70 (m, 3H), 2.34-2.16 (m, 1H), 2.12-2.04 (m, 1H), 1.92-1.84 (m, 1H), 1.40-1.14 (m, 2H); MS (ES, m/z) 389 M+H⁺ (Method B).

### Example 3o

### N-[5-(3,5-Difluoro-phenyl)-pyridin-2-yl]-2-((7S,8aR)-3-oxo-hexahydro-oxazolo[3,4-a]pyridin-7-yl)-acetamide

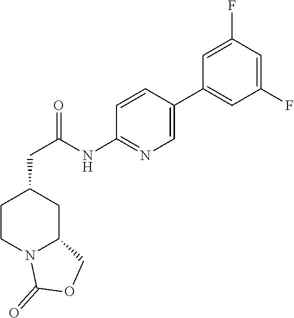

Prepared from 5-(3,5-difluoro-phenyl)-pyridin-2-ylamine and (3-oxo-hexahydro-oxazolo[3,4-a]pyridin-7-yl)-acetic acid. 1H NMR CDCl3 δ 9.62 (br, 1H), 8.44-8.36 (m, 2H), 7.94-7.89 (m, 1H), 7.10-7.04 (m, 2H), 6.88-6.80 (m, 1H), 4.45-4.40 (m, 1H), 3.98-3.88 (m, 2H), 3.82-3.72 (m, 1H), 3.00-2.90 (m, 1H), 2.50-2.34 (m, 2H), 2.30-2.12 (m, 1H), 2.04-1.96 (m, 1H), 1.86-1.79 (m, 1H), 1.34-1.14 (m, 2H); MS (ES, m/z) 388 M+H⁺ (Method B).

### Example 3p

### N-[5-(3,5-Difluoro-phenyl)-pyrazin-2-yl]-2-((7S,8aR)-3-oxo-hexahydro-oxazolo[3,4-a]pyridin-7-yl)-acetamide

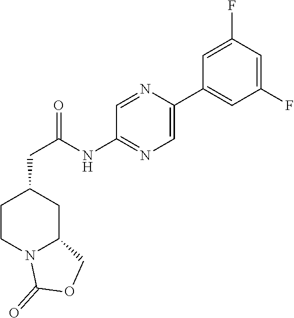

Prepared from 5-(3,5-difluoro-phenyl)-pyrazin-2-ylamine and ((7S,8aR)-3-Oxo-hexahydro-oxazolo[3,4-a]pyridin-7-yl)-acetic acid. 1H NMR CDCl3 δ 9.60-9.56 (m, 1H), 8.66-8.62 (m, 1H), 8.60-8.56 (br, 1H), 7.58-7.48 (m, 2H), 6.92-6.84 (m, 1H), 4.46-4.40 (m, 1H), 4.00-3.90 (m, 2H), 3.84-3.74 (m, 1H), 3.02-2.92 (m, 1H), 2.54-2.40 (m, 2H), 2.30-2.18 (m, 1H), 2.06-1.96 (m, 1H), 1.86-1.80 (m, 1H), 1.32-1.12 (m, 2H); MS (ES, m/z) 389 M+H⁺ (Method B).

### Example 3q

### N-[1-(2-Fluoro-phenyl)-1H-pyrazol-3-yl]-2-((7S,8aR)-3-oxo-hexahydro-oxazolo[3,4-a]pyridin-7-yl)-acetamide

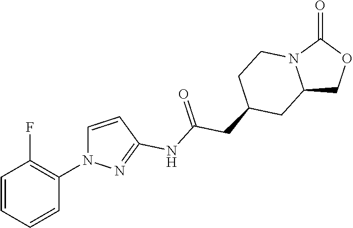

Prepared from 1-(2-fluoro-phenyl)-1H-pyrazol-3-yl lamine and ((7S,8aR)-3-oxo-hexahydro-oxazolo[3,4-a]pyridin-7-yl)-acetic acid. 1H NMR CDCl3 δ 9.46-9.42 (br, 1H), 7.90-7.87 (m, 1H), 7.76-7.70 (m, 1H), 7.30-7.18 (m, 3H), 6.99-6.96 (m, 1H), 4.38-4.32 (m, 1H), 3.86-3.78 (m, 2H), 3.70-3.60 (m, 1H), 2.88-2.78 (m, 1H), 2.16-1.96 (m, 3H), 1.84-1.76 (m, 1H), 1.64-1.56 (m, 1H), 1.00-0.76 (m, 2H); MS (ES, m/z) 359 M+H⁺ (Method B).

### Example 3r

### N-[5-(3,5-Difluoro-phenyl)-pyridin-2-yl]-2-((7S,8aR)-1,1-dimethyl-3-oxo-hexahydro-oxazolo[3,4-a]pyridin-7-yl)-acetamide

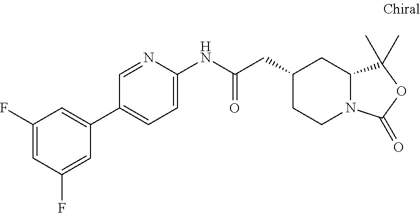

Prepared from 5-(3,5-difluoro-phenyl)-pyridin-2-ylamine and (1,1-dimethyl-3-oxo-hexahydro-oxazolo[3,4-a]pyridin-7-yl)-acetic acid, followed by chiral separation. 1H NMR CDCl3 δ 8.56-8.24 (m, 3H), 7.94-7.90 (m, 1H), 7.12-7.04 (m, 2H), 6.89-6.83 (m, 1H), 4.00-3.92 (m, 1H), 3.36-3.30 (m, 1H), 2.97-2.90 (m, 1H), 2.50-2.36 (m, 2H), 2.24-2.10 (m, 1H), 1.86-1.78 (m, 2H), 1.46 (s, 3H), 1.33 (s, 3H), 1.33-1.10 (m, 2H); MS (ES, m/z) 416 M+H⁺ (Method B).

### Example 3s

### N-[5-(3,5-Difluoro-phenyl)-pyridin-2-yl]-2-((7R,8aS)-1,1-dimethyl-3-oxo-hexahydro-oxazolo[3,4-a]pyridin-7-yl)-acetamide

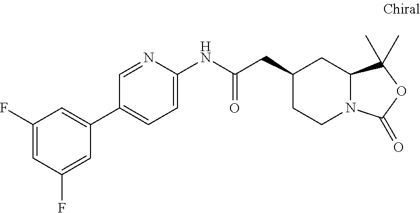

Prepared from 5-(3,5-difluoro-phenyl)-pyridin-2-ylamine and (1,1-dimethyl-3-oxo-hexahydro-oxazolo[3,4-a]pyridin-7-yl)-acetic acid, followed by chiral separation. 1H NMR CDCl3 δ 8.50-8.46 (m, 1H), 8.35-8.30 (m, 1H), 8.14 (br, 1H), 7.94-7.90 (m, 1H), 7.12-7.08 (m, 2H), 6.89-6.83 (m, 1H), 4.00-3.92 (m, 1H), 3.36-3.30 (m, 1H), 2.97-2.90 (m, 1H), 2.50-2.36 (m, 2H), 2.24-2.10 (m, 1H), 1.86-1.78 (m, 2H), 1.46 (s, 3H), 1.33 (s, 3H), 1.33-1.10 (m, 2H); MS (ES, m/z) 416 M+H⁺ (Method B).

### Example 3t

### N-[5-(3,5-Difluoro-phenyl)-pyrazin-2-yl]-2-(7S,8aR)-1,1-dimethyl-3-oxo-hexahydro-oxazolo[3,4-a]pyridin-7-yl)-acetamide

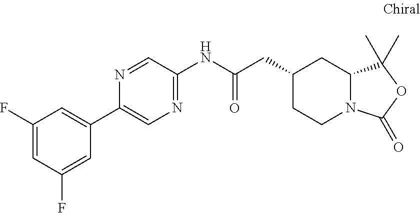

Prepared from 5-(3,5-difluoro-phenyl)-pyrazin-2-ylamine and (1,1-dimethyl-3-oxo-hexahydro-oxazolo[3,4-a]pyridin-7-yl)-acetic acid, followed by chiral separation. 1H NMR CDCl3 δ 9.60 (br, 1H), 8.65 (m, 1H), 8.33 (br, 1H), 7.58-7.50 (m, 2H), 6.93-6.84 (m, 1H), 4.00-3.92 (m, 1H), 3.36-3.30 (m, 1H), 2.98-2.88 (m, 1H), 2.57-2.41 (m, 2H), 2.26-2.14 (m, 1H), 1.88-1.78 (m, 2H), 1.46 (s, 3H), 1.32 (s, 3H), 1.32-1.10 (m, 2H); MS (ES, m/z) 417 M+H⁺ (Method B).

### Example 3u

### N-[5-(3,5-Difluoro-phenyl)-pyrazin-2-yl]-2-((7R,8aS)-1,1-dimethyl-3-oxo-hexahydro-oxazolo[3,4-a]pyridin-7-yl)-acetamide

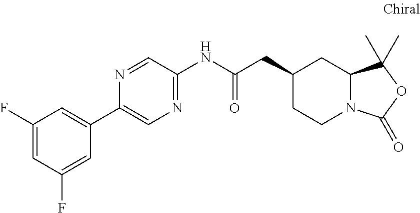

Prepared from 5-(3,5-difluoro-phenyl)-pyrazin-2-ylamine and (1,1-dimethyl-3-oxo-hexahydro-oxazolo[3,4-a]pyridin-7-yl)-acetic acid, followed by chiral separation. 1H NMR CDCl3 δ 9.60 (br, 1H), 8.65 (m, 1H), 8.40 (br, 1H), 7.58-7.50 (m, 2H), 6.93-6.84 (m, 1H), 4.00-3.92 (m, 1H), 3.36-3.30 (m, 1H), 2.98-2.88 (m, 1H), 2.57-2.41 (m, 2H), 2.26-2.14 (m, 1H), 1.88-1.78 (m, 2H), 1.46 (s, 3H), 1.32 (s, 3H), 1.32-1.10 (m, 2H); MS (ES, m/z) 417 M+H⁺ (Method B).

### Example 3v

### N-[5-(2,4-Difluoro-phenyl)-[1,3,4]thiadiazol-2-yl]-2-(1,1-dimethyl-3-oxo-hexahydro-oxazolo[3,4-a]pyridin-7-yl)-acetamide

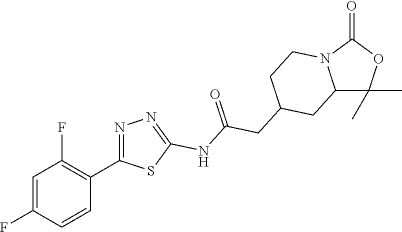

Prepared from 5-(2,4-difluoro-phenyl)-[1,3,4]thiadiazol-2-ylamine and (1,1-dimethyl-3-oxo-hexahydro-oxazolo[3,4-a]pyridin-7-yl)-acetic acid. 1H NMR CD3OD δ8.27-8.19 (m, 1H), 7.10-7.02 (m, 2H), 3.87-3.80 (m, 1H), 3.39-3.34 (m, 1H), 2.97-2.87 (m, 1H), 2.60-2.46 (m, 2H), 2.20-2.06 (m, 1H), 1.84-1.76 (m, 2H), 1.44 (s, 3H), 1.32 (s, 3H), 1.32-1.10 (m, 2H); MS (ES, m/z) 423 M+H⁺ (Method B).

### Example 3w

### N-[5-(2,4-Difluoro-phenyl)-[1,3,4]oxadiazol-2-yl]-2-(3-oxo-hexahydro-oxazolo[3,4-a]pyridin-7-yl)-acetamide

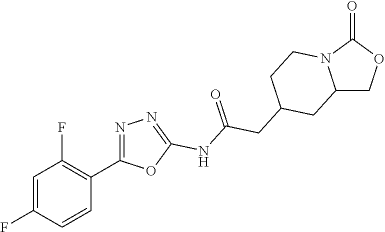

Prepared from 5-(2,4-difluoro-phenyl)-[1,3,4]oxadiazol-2-ylamine and (3-oxo-hexahydro-oxazolo[3,4-a]pyridin-7-yl)-acetic acid. 1H NMR CD3OD δ 8.06-7.98 (m, 1H), 7.13-7.04 (m, 2H), 4.48-4.42 (m, 1H), 3.96-3.76 (m, 3H), 3.02-2.92 (m, 1H), 2.54-2.40 (m, 2H), 2.33-2.10 (m, 1H), 2.04-1.96 (m, 1H), 1.86-1.77 (m, 1H), 1.32-1.10 (m, 2H); MS (ES, m/z) 379 M+H⁺ (Method B).

### Example 3x

### N-[1-(2,4-Difluoro-phenyl)-1H-pyrazol-3-yl]-2-((7S,8aR)-3-oxo-hexahydro-oxazolo[3,4-a]pyridin-7-yl)-acetamide

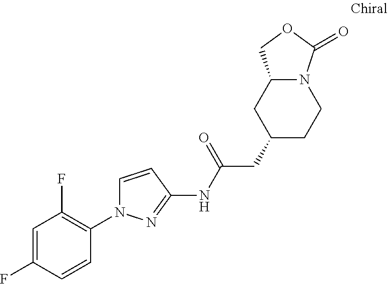

Prepared from 1-(2,4-difluoro-phenyl)-1H-pyrazol-3-ylamine and ((7S,8aR)-3-oxo-hexahydro-oxazolo[3,4-a]pyridin-7-yl)-acetic acid. 1H NMR CDCl3 δ 8.06-8.02 (br, 1H), 7.86-7.82 (m, 1H), 7.76-7.68 (m, 1H), 7.04-6.94 (m, 3H), 4.44-4.38 (m, 1H), 3.96-3.84 (m, 2H), 3.80-3.70 (m, 1H), 2.98-2.90 (m, 1H), 2.40-2.12 (m, 3H), 2.04-1.96 (m, 1H), 1.84-1.77 (m, 1H), 1.30-1.08 (m, 2H); MS (ES, m/z) 377 M+H⁺ (Method B).

### Example 3y

### N-[1-(3-Fluoro-phenyl)-1H-pyrazol-3-yl]-2-((7S,8aR)-3-oxo-hexahydro-oxazolo[3,4-a]pyridin-7-yl)-acetamide

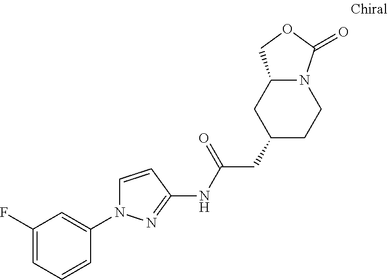

Prepared from 1-(3-fluoro-phenyl)-1H-pyrazol-3-ylamine and ((7S,8aR)-3-oxo-hexahydro-oxazolo[3,4-a]pyridin-7-yl)-acetic acid. 1H NMR CDCl3 δ 8.60 (br, 1H), 7.86-7.83 (m, 1H), 7.44-7.34 (m, 3H), 7.01-6.94 (m, 2H), 4.42-4.36 (m, 1H), 3.94-3.82 (m, 2H), 3.78-3.68 (m, 1H), 2.94-2.85 (m, 1H), 2.34-2.08 (m, 3H), 1.96-1.90 (m, 1H), 1.80-1.68 (m, 1H), 1.20-0.98 (m, 2H); MS (ES, m/z) 359 M+H⁺ (Method B).

### Example 3z

### N-[1-(4-Fluoro-phenyl)-1H-pyrazol-3-yl]-2-((7S,8aR)-3-oxo-hexahydro-oxazolo[3,4-a]pyridin-7-yl)-acetamide

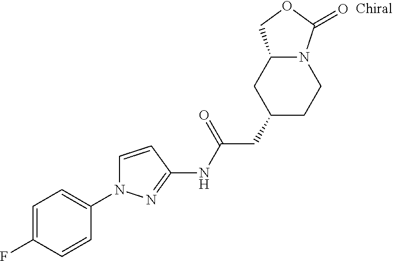

Prepared from 1-(4-fluoro-phenyl)-1H-pyrazol-3-ylamine and ((7S,8aR)-3-oxo-hexahydro-oxazolo[3,4-a]pyridin-7-yl)-acetic acid. 1H NMR CDCl3 δ 8.17 (br, 1H), 7.78-7.76 (m, 1H), 7.59-7.32 (m, 2H), 7.17-7.11 (m, 2H), 6.94-6.92 (m, 1H), 4.43-4.37 (m, 1H), 3.95-3.82 (m, 2H), 3.78-3.70 (m, 1H), 2.97-2.88 (m, 1H), 2.38-2.10 (m, 3H), 2.01-1.94 (m, 1H), 1.82-1.74 (m, 1H), 1.30-1.05 (m, 2H); MS (ES, m/z) 359 M+H⁺ (Method B).

### Example 3aa

### N-[1-(3,5-Difluoro-phenyl)-1H-pyrazol-3-yl]-2-((7S,8aR)-3-oxo-hexahydro-oxazolo[3,4-a]pyridin-7-yl)-acetamide

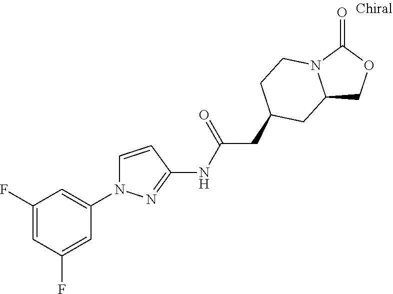

Prepared from 1-(3,5-difluoro-phenyl)-1H-pyrazol-3-ylamine and ((7S,8aR)-3-oxo-hexahydro-oxazolo[3,4-a]pyridin-7-yl)-acetic acid. 1H NMR CDCl3 δ 8.31 (br, 1H), 7.84-7.78 (m, 1H), 7.22-7.10 (m, 2H), 7.02-6.96 (m, 1H), 6.74-6.66 (m, 1H), 4.46-4.37 (m, 1H), 3.98-3.68 (m, 3H), 2.99-2.88 (m, 1H), 2.44-2.08 (m, 3H), 2.03-1.92 (m, 1H), 1.84-1.70 (m, 1H), 1.32-1.06 (m, 2H); MS (ES, m/z) 377 M+H⁺ (Method B).

### Example 3bb

### N-[1-(2,5-Difluoro-phenyl)-1H-pyrazol-3-yl]-2-((7S,8aR)-3-oxo-hexahydro-oxazolo[3,4-a]pyridin-7-yl)-acetamide

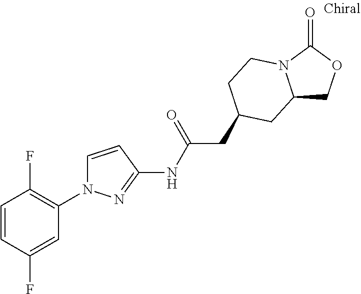

Prepared from 1-(2,5-difluoro-phenyl)-1H-pyrazol-3-ylamine and ((7S,8aR)-3-oxo-hexahydro-oxazolo[3,4-a]pyridin-7-yl)-acetic acid. 1H NMR CDCl3 δ 8.34 (br, 1H), 8.02-7.96 (m, 1H), 7.60-7.52 (m, 1H), 7.24-7.14 (m, 1H), 7.02-6.88 (m, 2H), 4.46-4.37 (m, 1H), 3.98-3.68 (m, 3H), 2.98-2.87 (m, 1H), 2.40-2.10 (m, 3H), 2.03-1.92 (m, 1H), 1.84-1.70 (m, 1H), 1.30-1.04 (m, 2H); MS (ES, m/z) 377 M+H⁺ (Method B).

### Example 3 cc

### N-[1-(4-Chloro-2-fluoro-phenyl)-1H-pyrazol-3-yl]-2-((7S,8aR)-3-oxo-hexahydro-oxazolo[3,4-a]pyridin-7-yl)-acetamide

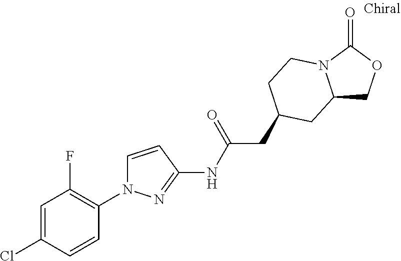

Prepared from 1-(4-chloro-2-fluoro-phenyl)-1H-pyrazol-3-ylamine and ((7S,8aR)-3-oxo-hexahydro-oxazolo[3,4-a]pyridin-7-yl)-acetic acid. 1H NMR CDCl3 δ 8.28 (br, 1H), 7.90-7.88 (m, 1H), 7.75-7.69 (m, 1H), 7.29-7.20 (m, 2H), 6.98-6.96 (m, 1H), 4.43-4.37 (m, 1H), 3.96-3.84 (m, 2H), 3.78-3.68 (m, 1H), 2.97-2.88 (m, 1H), 2.38-2.10 (m, 3H), 2.00-1.92 (m, 1H), 1.82-1.74 (m, 1H), 1.28-1.05 (m, 2H); MS (ES, m/z) 393 M+H⁺ (Method B).

### Example 3dd

### N-[1-(2-Ethoxy-6-fluoro-phenyl)-1H-pyrazol-3-yl]-2-((7S,8aR)-3-oxo-hexahydro-oxazolo[3,4-a]pyridin-7-yl)-acetamide

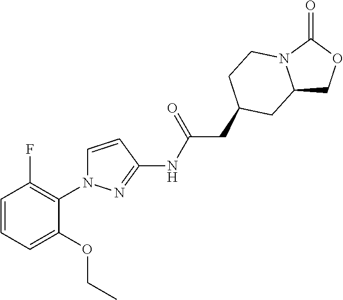

Prepared from 1-(2-fluoro-6-ethoxyl-phenyl)-1H-pyrazol-3-ylamine and ((7S,8aR)-3-oxo-hexahydro-oxazolo[3,4-a]pyridin-7-yl)-acetic acid. 1H NMR CDCl3 δ 8.75 (br, 1H), 7.53-7.49 (m, 1H), 7.39-7.28 (m, 1H), 6.95-6.90 (m, 1H), 6.88-6.78 (m, 2H), 4.43-4.36 (m, 1H), 4.10-4.00 (m, 2H), 3.94-3.82 (m, 2H), 3.78-3.66 (m, 1H), 2.96-2.84 (m, 1H), 2.28-1.88 (m, 4H), 1.76-1.66 (m, 1H), 1.34-1.24 (m, 3H), 1.20-0.93 (m, 2H); MS (ES, m/z) 403 M+H⁺ (Method B).

### Example 3ee

### N-[1-(2,3-Difluoro-phenyl)-1H-pyrazol-3-yl]-2-((7S,8aR)-3-oxo-hexahydro-oxazolo[3,4-a]pyridin-7-yl)-acetamide

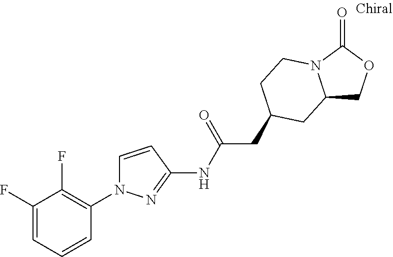

Prepared from 1-(2,3-difluoro-phenyl)-1H-pyrazol-3-ylamine and ((7S,8aR)-3-oxo-hexahydro-oxazolo[3,4-a]pyridin-7-yl)-acetic acid. 1H NMR CDCl3 δ 8.12 (br, 1H), 7.96-7.90 (m, 1H), 7.60-7.50 (m, 1H), 7.22-7.04 (m, 2H), 7.02-6.96 (m, 1H), 4.46-4.37 (m, 1H), 3.98-3.68 (m, 3H), 2.98-2.86 (m, 1H), 2.40-2.10 (m, 3H), 2.03-1.92 (m, 1H), 1.84-1.74 (m, 1H), 1.30-1.04 (m, 2H); MS (ES, m/z) 377 M+H⁺ (Method B).

### Example 3ff

### N-[1-(3,5-Difluoro-phenyl)-1H-imidazol-4-yl]-2-((7S,8aR)-3-oxo-hexahydro-oxazolo[3,4-a]pyridin-7-yl)-acetamide

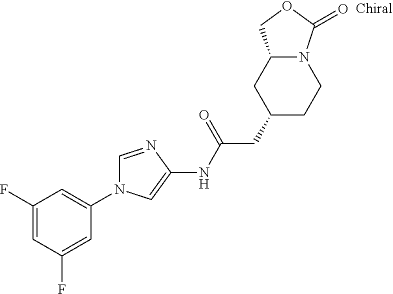

Prepared from 1-(3,5-difluoro-phenyl)-1H-imidazol-4-ylamine and ((7S,8aR)-3-oxo-hexahydro-oxazolo[3,4-a]pyridin-7-yl)-acetic acid. 1H NMR CDCl₃ δ 9.42 (br, 1H), 8.08-7.70 (m, 2H), 7.06-6.98 (m, 2H), 6.92-6.86 (m, 1H), 4.44-4.38 (m, 1H), 3.98-3.70 (m, 3H), 2.98-2.88 (m, 1H), 2.48-2.10 (m, 3H), 2.02-1.94 (m, 1H), 1.84-1.76 (m, 1H), 1.36-1.14 (m, 2H); MS (ES, m/z) 377 M+H⁺ (Method B).

### Example 3gg

### N-[1-(2-Chloro-phenyl)-1H-pyrazol-3-yl]-2-((7S,8aR)-3-oxo-hexahydro-oxazolo[3,4-a]pyridin-7-yl)-acetamide

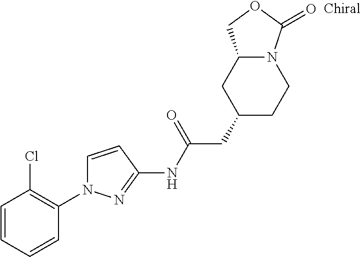

Prepared from 1-(2-chloro-phenyl)-1H-pyrazol-3-ylamine and ((7S,8aR)-3-oxo-hexahydro-oxazolo[3,4-a]pyridin-7-yl)-acetic acid. 1H NMR CDCl3 δ 9.01 (br, 1H), 7.82-7.79 (m, 1H), 7.57-7.50 (m, 2H), 7.42-7.36 (m, 2H), 7.00-6.97 (m, 1H), 4.42-4.34 (m, 1H), 3.92-3.82 (m, 2H), 3.78-3.64 (m, 1H), 2.94-2.82 (m, 1H), 2.20-1.96 (m, 3H), 1.92-1.84 (m, 1H), 1.72-1.62 (m, 1H), 1.10-0.84 (m, 2H); MS (ES, m/z) 375 M+H⁺ (Method B).

### Example 3hh

### 2-((7S,8aR)-3-oxo-hexahydro-oxazolo[3,4-a]pyridin-7-yl)-N-[3-(3-trifluoromethyl-phenyl)-isoxazol-5-yl]-acetamide

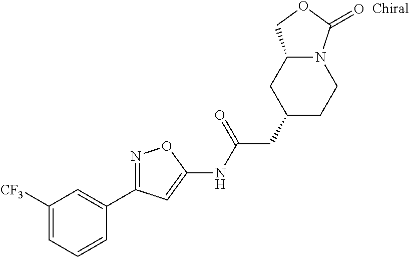

Prepared from 3-(3-trifluoromethyl-phenyl)-isoxazol-5-ylamine and ((7S,8aR)-3-oxo-hexahydro-oxazolo[3,4-a]pyridin-7-yl)-acetic acid. 1H NMR CDCl3 δ 8.80-8.72 (br, 1H), 8.09-8.06 (m, 1H), 8.02-7.98 (m, 1H), 7.74-7.70 (m, 1H), 7.64-7.58 (m, 1H), 6.79-6.75 (m, 1H), 4.46-4.42 (m, 1H), 4.00-3.88 (m, 2H), 3.84-3.74 (m, 1H), 3.02-2.92 (m, 1H), 2.52-2.38 (m, 2H), 2.30-2.16 (m, 1H), 2.04-1.96 (m, 1H), 1.86-1.78 (m, 1H), 1.35-1.15 (m, 2H); MS (ES, m/z) 410 M+H⁺ (Method B).

### Example 3ii

### N-[1-(2-Chloro-4-ethoxy-phenyl)-1H-pyrazol-3-yl]-2-((7S,8aR)-3-oxo-hexahydro-oxazolo[3,4-a]pyridin-7-yl)-acetamide

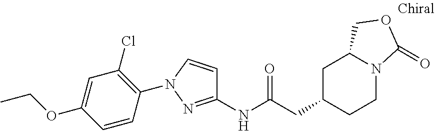

Prepared from 1-(2-chloro-4-ethoxyl-phenyl)-1H-pyrazol-3-ylamine and ((7S,8aR)-3-oxo-hexahydro-oxazolo[3,4-a]pyridin-7-yl)-acetic acid. 1H NMR CDCl3 δ 9.06-9.02 (br, 1H), 7.68-7.64 (br, 1H), 7.42-7.36 (m, 1H), 7.04-7.02 (m, 1H), 6.94-6.86 (m, 2H), 4.41-4.34 (m, 1H), 4.10-4.05 (m, 2H), 3.92-3.82 (m, 2H), 3.78-3.66 (m, 1H), 2.92-2.84 (m, 1H), 2.16-1.64 (m, 5H), 1.48-1.42 (m, 3H), 1.16-0.88 (m, 2H); MS (ES, m/z) 419 M+H⁺ (Method B).

### Example 3jj

### N-[3-(2,4-Difluoro-phenyl)-isoxazol-5-yl]-2-((7S,8aR)-3-oxo-hexahydro-oxazolo[3,4-a]pyridin-7-yl)-acetamide

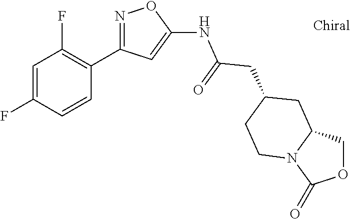

Prepared from 3-(2,4-difluoro-phenyl)-isoxazol-5-ylamine and ((7S,8aR)-3-oxo-hexahydro-oxazolo[3,4-a]pyridin-7-yl)-acetic acid. 1H NMR CDClδ 8.84-8.78 (br, 1H), 7.95-7.88 (m, 1H), 7.02-6.92 (m, 2H), 6.82-6.76 (m, 1H), 4.47-4.41 (m, 1H), 4.00-3.88 (m, 2H), 3.84-3.74 (m, 1H), 3.02-2.92 (m, 1H), 2.52-2.38 (m, 2H), 2.30-2.16 (m, 1H), 2.04-1.96 (m, 1H), 1.86-1.78 (m, 1H), 1.35-1.14 (m, 2H); MS (ES, m/z) 378 M+H⁺ (Method B).

### Example 3kk

### N-[1-(2-Chloro-4-fluoro-phenyl)-1H-pyrazol-3-yl]-2-((7S,8aR)-3-oxo-hexahydro-oxazolo[3,4-a]pyridin-7-yl)-acetamide

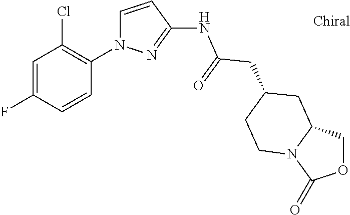

Prepared from (2-chloro-4-fluoro-phenyl)-1H-pyrazol-3-ylamine and ((7S,8aR)-3-oxo-hexahydro-oxazolo[3,4-a]pyridin-7-yl)-acetic acid. 1H NMR CDCl3 δ 8.54-8.50 (br, 1H), 7.73-7.69 (m, 1H), 7.50-7.44 (m, 1H), 7.30-7.26 (m, 1H), 7.14-7.07 (m, 1H), 6.98-6.94 (m, 1H), 4.44-4.36 (m, 1H), 3.95-3.84 (m, 2H), 3.81-3.70 (m, 1H), 2.96-2.87 (m, 1H), 2.35-2.10 (m, 3H), 2.00-1.94 (m, 1H), 1.82-1.74 (m, 1H), 1.30-1.14 (m, 2H); MS (ES, m/z) 393 M+H⁺ (Method B).

### Preparation of the Compounds of Formula I

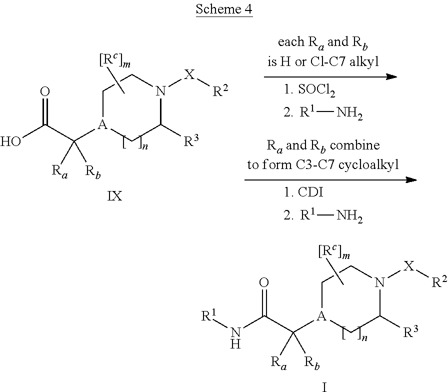

Moreover, the compounds of Formula I may also be synthesized from an acid of Formula IX as described in Scheme 4. The acid may be activated using either thionyl chloride or CDI.

### Intermediate of Formula IXd: 1-(1-acetyl-piperidin-4-yl)-cyclopropanecarboxylic acid

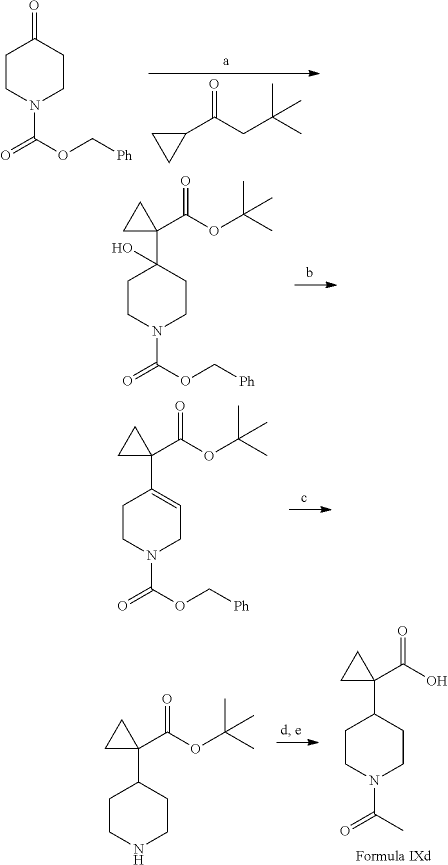

a. LDA, THF, about −78° C. 1 h then about 0° C. about 0.5 h. b. Burgess reagent, toluene, about 90° C., about 0.5 h. c. H₂, Pd(OH)₂/C, EtOAC, MeOH, RT overnight. d. Acetyl chloride, triethylamine, DCM, RT for about 1.5 h. e. TFA, DCM, RT for about 1 h.

Step 1 4-(1-tert-Butoxycarbonyl-cyclopropyl)-4-hydroxy-piperidine-1-carboxylic acid benzyl ester: To a cooled (−78° C.) solution of 2.0M lithium diisopropylamide in heptane/THF/ethylbenzene (27.4 mL, 54.8 mmol) was added cyclopropanecarboxylic acid tert-butyl ester (7.08 g, 49.8 mmol) in THF (35 mL). After stirring at −78° C. for 1 h, a solution of 4-oxo-piperidine-1-carboxylic acid benzyl ester (11.6 g, 49.8 mmol) in THF (50 mL) was added. The reaction mixture was stirred at −78° C. for 3 h then warmed to room temperature. After stirring at ambient temperature for 16 h, the mixture was quenched with saturated aqueous solution of NH₄Cl (150 mL) and extracted with EtOAc (150 mL×3). The combined organic phase was washed with brine, dried over sodium sulfate, filtered and concentrated in vacuo. The residue was purified by silica gel chromatography (0-40% EtOAc in Hexane) to yield 4-(1-tert-butoxycarbonyl-cyclopropyl)-4-hydroxy-piperidine-1-carboxylic acid benzyl ester (8.07 g, 43%). 1H NMR (400 MHz, CDCl₃) δ 7.36-7.28 (m, 5H), 5.11 (s, 2H), 4.50 (s, 1H), 4.00 (brs, 2H), 3.20 (brs, 2H), 1.72-1.64 (m, 2H), 1.42 (s, 9H), 1.40-1.30 (m, 2H), 1.10 (dd, J=7.0 and 2.4 Hz, 2H), 0.88 (dd, J=7.0 and 2.4 Hz, 2H).

Step 2 4-(1-tert-Butoxycarbonyl-cyclopropyl)-3,6-dihydro-2H-pyridine-1-carboxylic acid benzyl ester: A mixture of 4-(1-tert-butoxycarbonyl-cyclopropyl)-4-hydroxy-piperidine-1-carboxylic acid benzyl ester (5.41 g, 14.4 mmol) and (methoxycarbonylsulfamoyl)-triethylammonium hydroxide (4.1 g, 17 mmol) in toluene (100 mL) was heated at 90° C. for 1 h. The reaction mixture was quenched with water (50 mL) and extracted with EtOAc (150 mL×3). The combined organic phase was washed with saturated aqueous solution of NaHCO₃ and brine, dried over sodium sulfate, filtered and concentrated in vacuo. The residue was purified by silica gel chromatography (0-20% EtOAc in Hexane) to yield 4-(1-tert-butoxycarbonyl-cyclopropyl)-3,6-dihydro-2H-pyridine-1-carboxylic acid benzyl ester (4.32 g, 84%). ¹H NMR (400 MHz, CDCl₃) δ 7.38-7.30 (m, 5H), 5.51 (m, 2H), 5.15 (s, 2H), 3.96 (m, 2H), 3.59 (t, J=5.3 Hz, 2H), 2.25 (brs, 2H), 1.41 (s, 9H), 1.24 (dd, J=6.6 and 3.0 Hz, 2H), 0.85 (dd, J=6.6 and 3.0 Hz, 2H).

Step 3 1-Piperidin-4-yl-cyclopropanecarboxylic acid tert-butyl ester: A solution of 4-(1-tert-butoxycarbonyl-cyclopropyl)-3,6-dihydro-2H-pyridine-1-carboxylic acid benzyl ester (4.32 g, 12.1 mmol) and 20% palladium hydroxide on charcoal (2:8, Palladium:carbon black, 1.70 g) in methanol (70 mL) and EtOAc (70 mL) was stirred under an atmosphere of hydrogen (1 atm) for overnight. The reaction mixture was filtered and concentrated in vacuo to yield the desired product (2.66 g, 98%). ¹H NMR (400 MHz, CDCl₃) δ 6.93 (S, 1H), 3.22 (d, J=12.2 Hz, 2H), 2.63 (dt, J=12.5 and 2.2 Hz, 1H), 1.80-1.70 (m, 1H), 1.66-1.56 (m, 2H), 1.56-1.40 (m, 2H), 1.34 (s, 9H), 0.98 (dd, J=6.8 and 2.6 Hz, 2H), 0.62 (dd, J=6.8 and 2.6 Hz, 2H). ESI-MS m/z: 226.1 (M+H)⁺.

Step 4 1-(1-Acetyl-piperidin-4-yl)-cyclopropanecarboxylic acid tert-butyl ester: To a cooled (0° C.) solution of 1-piperidin-4-yl-cyclopropanecarboxylic acid tert-butyl ester (2.66 g, 11.8 mmol) and triethylamine (3.29 mL, 23.6 mmol) in methylene chloride (80 mL) was added acetyl chloride (1.26 mL, 17.7 mmol). The reaction mixture was warmed to room temperature and stirred for 4 h. The mixture was quenched with saturated aqueous solution of NH₄Cl (80 mL) and extracted with methylene chloride (50 mL×3). The combined organic phase was washed with saturated aqueous solution of NaHCO₃ and brine, dried over sodium sulfate, filtered and concentrated in vacuo to afford 1-(1-Acetyl-piperidin-4-yl)-cyclopropanecarboxylic acid tert-butyl ester (3.13 g, 99%). ¹H NMR (400 MHz, CDCl₃) δ 4.69-4.65 (m, 1H), 3.84-3.79 (m, 1H), 2.98 (dt, J=12.0 and 2.5 Hz, 1H), 2.43 (dt, J=12.0 and 2.5 Hz, 1H), 2.07 (s, 3H), 1.74-1.65 (m, 2H), 1.63-1.56 (m, 1H), 1.50-1.38 (m, 2H), 1.41 (s, 9H), 1.10-1.04 (m, 2H), 0.70-0.60 (m, 2H).

Step 5 1-(1-Acetyl-piperidin-4-yl)-cyclopropanecarboxylic acid: To a cooled (0° C.) solution of 1-(1-acetyl-piperidin-4-yl)-cyclopropanecarboxylic acid tert-butyl ester (3.13 g, 11.7 mmol) and triethylsilane (4.67 mL, 29.3 mmol) in methylene chloride (25 mL) was added trifluoroacetic acid (11.7 mL, 152 mmol). The reaction mixture was warmed to room temperature and stirred for 1 h. The volatiles were removed in vacuo. The residue was dissolved in methylene chloride (150 mL) and extracted with 2M NaOH (75 mL). The aqueous layer was acidified with 12N HCl and extracted with methylene chloride (150 mL×3). The combined organic phase was washed with brine, dried over sodium sulfate, filtered and concentrated in vacuo to afford 1-(1-acetyl-piperidin-4-yl)-cyclopropanecarboxylic acid (1.71 g, 69%). ¹H NMR (400 MHz, CDCl₃) δ 4.69 (d, J=12.0 Hz, 1H), 3.83 (d, J=12.0 Hz, 1H), 2.99 (t, J=12.0 Hz, 1H), 2.44 (t, J=12.0 Hz, 1H), 2.10 (s, 3H), 1.74-1.61 (m, 3H), 1.57-1.41 (m, 2H), 1.26-1.22 (m, 2H), 0.79-0.77 (m, 2H). ESI-MS m/z: 212.1 (M+H)⁺.

### Intermediate of Formula IXe: 2-(1-Acetyl-piperidin-4-yl)-2-methyl-propionic acid

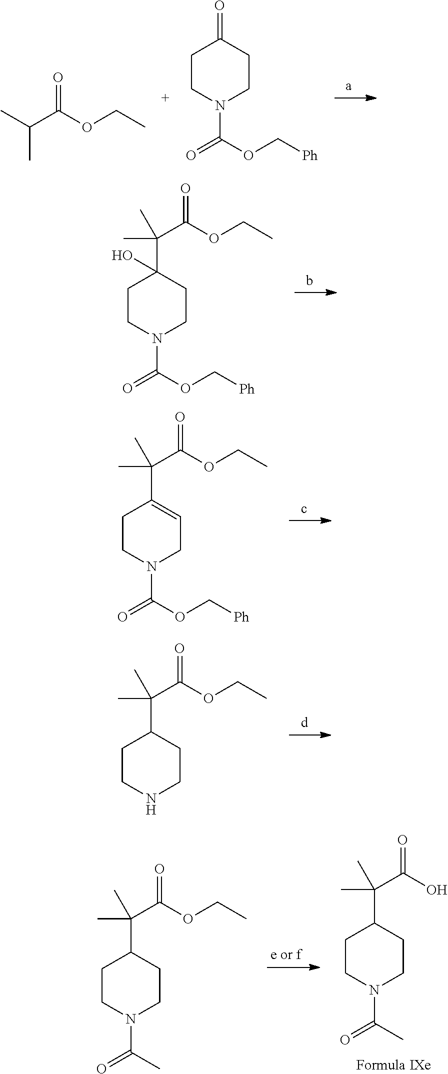

a. LDA, THF, −78° C. 1 h then 0° C. a. 5 h. b. Burgess reagent, toluene, 90° C. 0.5 h. (c) H₂, Pd(OH)₂/C, EtOAC, MeOH, rt overnight. (d) Acetyl chloride, triethylamine, DCM, rt for 1.5 h. (e) NaOH, MeOH, reflux overnight. (f) KOC(CH₃)₃, H₂O, ether, rt 4 days.

Step 1: 4-(1-Ethoxycarbonyl-1-methyl-ethyl)-4-hydroxy-piperidine-1-carboxylic acid benzyl ester. To a cooled (−78° C.) solution of 2.0M lithium diisopropylamide in heptane/THF/ethylbenzene (13.7 mL, 27.4 mmol) in THF (50 mL) was added isobutyric acid ethyl ester (3.32 mL, 24.9 mmol) in THF (20 mL). After stirring at −78° C. for 1 h, a solution of 4-oxo-piperidine-1-carboxylic acid benzyl ester (5.80 g, 24.9 mmol) in THF (50 mL) was added. After stirring at −78° C. for 2 h, the mixture was quenched with saturated aqueous solution of NH₄Cl (80 mL) and extracted with EtOAc (100 mL×3). The combined organic phase was washed with brine, dried over sodium sulfate, filtered and concentrated in vacuo. The residue was purified by silica gel chromatography (0-30% EtOAc in hexane) to yield 4-(1-ethoxycarbonyl-1-methyl-ethyl)-4-hydroxy-piperidine-1-carboxylic acid benzyl ester (6.91 g, 79%). ¹H NMR (400 MHz, CDCl₃) δ 7.37-7.28 (m, 5H), 5.13 (s, 2H), 4.17 (q, J=7.2 Hz, 2H), 4.05 (brs, 2H), 3.74 (s, 1H), 3.20 (brs, 2H), 1.62 (brs, 2H), 1.50-1.40 (m, 2H), 1.28 (t, J=7.2 Hz, 3H), 1.21 (s, 6H).

Step 2: 4-(1-Ethoxycarbonyl-1-methyl-ethyl)-3,6-dihydro-2H-pyridine-1-carboxylic acid benzyl ester. A mixture of 4-(1-ethoxycarbonyl-1-methyl-ethyl)-4-hydroxy-piperidine-1-carboxylic acid benzyl ester (1.90 g, 5.44 mmol) and (methoxycarbonylsulfamoyl)-triethylammonium hydroxide (1.60 g, 6.50 mmol) in toluene (30 mL) was heated at 90° C. for 1 h. The reaction mixture was quenched with water (50 mL) and extracted with EtOAc (150 mL×3). The combined organic phase was washed with saturated aqueous solution of NaHCO₃ and brine, dried over sodium sulfate, filtered and concentrated in vacuo. The residue was purified by silica gel chromatography (0-20% EtOAc in Hexane) to yield 4-(1-ethoxycarbonyl-1-methyl-ethyl)-3,6-dihydro-2H-pyridine-1-carboxylic acid benzyl ester (1.49 g, 83%). ¹H NMR (400 MHz, CDCl₃) δ 7.38-7.30 (m, 5H), 5.55 (m, 1H), 5.15 (s, 2H), 4.12 (q, J=7.1 Hz, 2H), 4.01 (m, 2H), 3.54 (t, J=5.4 Hz, 2H), 2.08 (brs, 2H), 1.30 (s, 6H), 1.22 (t, J=7.1 Hz, 3H).

Step 3: 2-Methyl-2-piperidin-4-yl-propionic acid ethyl ester. A solution of 4-(1-ethoxycarbonyl-1-methyl-ethyl)-3,6-dihydro-2H-pyridine-1-carboxylic acid benzyl ester (8.64 g, 26.1 mmol) and 20% palladium hydroxide on charcoal (2:8, Palladium:carbon black, 0.93 g) in methanol (100 mL) and EtOAc (100 mL) was stirred under an atmosphere of hydrogen (1 atm) overnight. The reaction mixture was filtered and concentrated to yield the desired product (4.93 g, 95%). ¹H NMR (400 MHz, CDCl₃) δ 4.09 (q, J=7.1 Hz, 2H), 3.15-3.05 (m, 2H), 2.65-2.50 (m, 3H), 1.68 (tt, J=12.2 and 3.3 Hz, 1H), 1.54-1.46 (m, 2H), 1.30-1.20 (m, 2H), 1.22 (t, J=7.1 Hz, 3H), 1.09 (s, 6H).

Step 4: 2-(1-Acetyl-piperidin-4-yl)-2-methyl-propionic acid ethyl ester. To a cooled (0° C.) solution of 2-methyl-2-piperidin-4-yl-propionic acid ethyl ester (813 mg, 4.08 mmol) and triethylamine (1.14 mL, 8.16 mmol) in methylene chloride (20 mL) was added acetyl chloride (0.44 mL, 6.12 mmol). The reaction mixture was warm up to room temperature and stirred for 1 h. The mixture was quenched with 1N HCl (30 mL) and extracted with methylene chloride (50 mL×3). The combined organic phase was washed with saturated aqueous solution of NaHCO₃ and brine, dried over sodium sulfate, filtered and concentrated in vacuo to afford 2-(1-acetyl-piperidin-4-yl)-2-methyl-propionic acid ethyl ester (980 mg, 99%). ¹H NMR (400 MHz, CDCl₃) δ 4.75-4.65 (m, 1H), 4.13 (q, J=7.1 Hz, 2H), 3.90-3.80 (m, 1H), 3.00 (dt, J=13.1 and 2.4 Hz, 1H), 2.50-2.40 (m, 1H), 2.08 (s, 3H), 1.85-1.75 (m, 1H), 1.65-1.55 (m, 2H), 1.30-1.15 (m, 2H), 1.24 (t, J=7.1 Hz, 3H), 1.10 (d, J=5.0 Hz, 6H).

Step 5: 2-(1-Acetyl-piperidin-4-yl)-2-methyl-propionic acid. To a cooled (0° C.) stirred suspension of potassium tert-butoxide (8.07 g, 71.9 mmol) in ether (100 mL) was added water (0.32 mL, 18.0 mmol). After stirring at 0° C. for 5 minutes, a solution of 2-(1-acetyl-piperidin-4-yl)-2-methyl-propionic acid ethyl ester (2.17 g, 8.99 mmol) in ether (50 mL) was added. The reaction mixture was warm up to room temperature and stirred for 4 days. The mixture was quenched with ice water (50 mL). The aqueous layer was acidified with 12N HCl (6.5 mL) and extracted with methylene chloride (100 mL×3). The combined organic phase was washed with brine, dried over sodium sulfate, filtered and concentrated in vacuo to afford 2-(1-acetyl-piperidin-4-yl)-2-methyl-propionic acid (1.50 g, 78%). ¹H NMR (400 MHz, CDCl₃) δ 4.75-4.65 (m, 1H), 3.90-3.80 (m, 1H), 3.02 (dt, J=12.9 and 2.3 Hz, 1H), 2.48 (dt, J=13.0 and 2.4 Hz, 1H), 2.10 (s, 3H), 1.88-1.78 (m, 1H), 1.72-1.62 (m, 2H), 1.34-1.20 (m, 2H), 1.16 (d, J=5.2 Hz, 6H).

### Compounds of the Invention

The following compounds were prepared according to the procedures in Scheme 4.

### Example 4a

### 2-(1-Acetyl-piperidin-4-yl)-N-[5-(3,5-difluoro-phenyl)-pyridin-2-yl]-isobutyramide

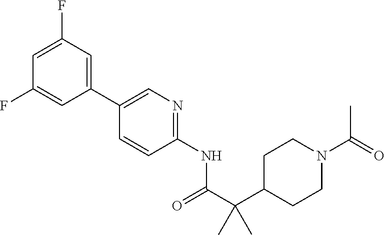

To a cooled (0° C.) solution of 2-(1-acetyl-piperidin-4-yl)-2-methyl-propionic acid (65 mg, 0.30 mmol) in methylene chloride (2 mL) was added thionyl chloride (26.7 uL, 0.37 mmol). After stirring at 0° C. for 30 min, a solution of 5-(3,5-difluoro-phenyl)-pyridin-2-ylamine in methylene chloride (2 mL) was added. The reaction mixture was heated to reflux overnight. The mixture was concentrated in vacuo. The residue was purified by HPLC to afford 2-(1-acetyl-piperidin-4-yl)-N-[5-(3,5-difluoro-phenyl)-pyridin-2-yl]-isobutyramide (14 mg, 11%). 1H NMR (400 MHz, CDCl₃) δ 8.47 (d, J=2.2 Hz, 1H), 8.35 (dd, J=8.6 and 0.6 Hz, 1H), 8.12 (s, 1H), 7.88 (dd, J=8.7 and 2.5 Hz, 1H), 7.09-7.05 (m, 2H), 6.83 (tt, J=8.8 and 2.3 Hz, 1H), 4.76-4.66 (m, 1H), 3.89-3.84 (m, 1H), 3.04 (dt, J=12.9 and 2.5 Hz, 1H), 2.49 (dt, J=13.0 and 2.6 Hz, 1H), 2.08 (s, 3H), 2.00-1.90 (m, 1H), 1.75-1.60 (m, 2H), 1.35-1.20 (m, 2H), 1.28 (d, J=4.7 Hz, 6H). ESI-MS m/z: 402.0 (M+H)⁺.

Likewise, the following compounds were prepared analogously to that of Example 4a.

### Example 4b

### 2-(1-Acetyl-piperidin-4-yl)-N-(3′,5′-difluoro-biphenyl-4-yl)-propionamide

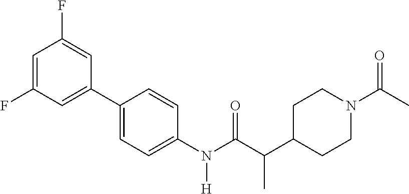

Prepared from 2-(1-acetyl-piperidin-4-yl)-propionic acid and 3′,5′-difluoro-biphenyl-4-ylamine. LC-MS (m/z) 387.0 (MH⁺); t_{R}=1.26 min (Method A).

### Example 4c

### (R)-2-(1-Acetyl-piperidin-4-yl)-N-(3′,5′-difluoro-biphenyl-4-yl)-propionamide

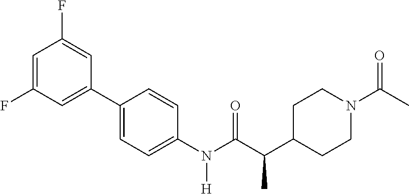

Prepared from (R)-2-(1-acetyl-piperidin-4-yl)-propionic acid and 3′,5′-difluoro-biphenyl-4-ylamine. LC-MS (m/z) 387.0 (MH⁺); t_{R}=1.24 min (Method A).

### Example 4d

### (S)-2-(1-Acetyl-piperidin-4-yl)-N-(3′,5′-difluoro-biphenyl-4-yl)-propionamide

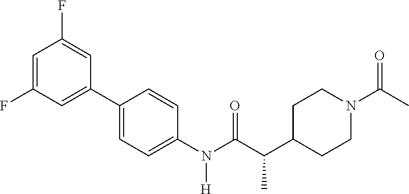

Prepared from (S)-2-(1-acetyl-piperidin-4-yl)-propionic acid and 3′,5′-difluoro-biphenyl-4-ylamine. LC-MS (m/z) 387.0 (MH⁺); t_{R}=1.24 min (Method A).

### Example 4e

### 2-(1-Acetyl-piperidin-4-yl)-N-[5-(3,5-difluoro-phenyl)-pyridin-2-yl]-propionamide

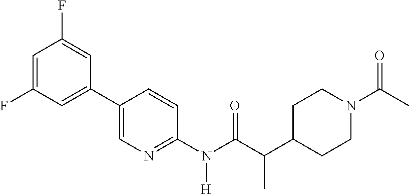

Prepared from 2-(1-acetyl-piperidin-4-yl)-propionic acid and 5-(3,5-difluoro-phenyl)-pyridin-2-ylamine. LC-MS (m/z) 388.0 (M); t_{R}=1.18 min (Method A).

### Example 4f

### 2-(1-Acetyl-piperidin-4-yl)-N-[5-(3,5-difluoro-phenyl)-pyridin-2-yl]-isobutyramide

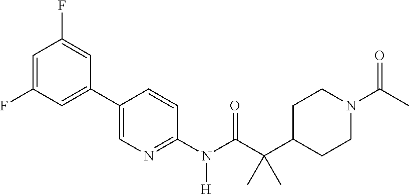

Prepared from 2-(1-acetyl-piperidin-4-yl)-2-methyl-propionic acid and 5-(3,5-difluoro-phenyl)-pyridin-2-ylamine. LC-MS (m/z) 402.0 (MH⁺); t_{R}=1.27 min (Method A).

### Example 4g

### 2-(1-Acetyl-piperidin-4-yl)-N-[5-(3,5-difluoro-phenyl)-pyrazin-2-yl]-isobutyramide

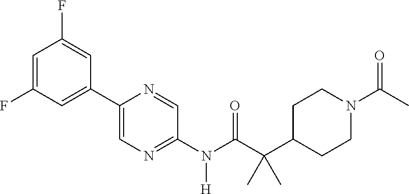

Prepared from 2-(1-acetyl-piperidin-4-yl)-2-methyl-propionic acid and 5-(3,5-difluoro-phenyl)-pyrazin-2-ylamine. LC-MS (m/z) 403.0 (MH⁺); t_{R}=1.24 min (Method A).

### Example 4h

### 1-(1-Acetyl-piperidin-4-yl)-cyclopropanecarboxylic acid [5-(3,5-difluoro-phenyl)-pyridin-2-yl]amide

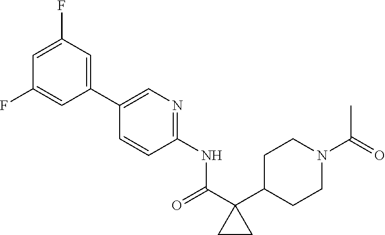

A solution of 1-(1-acetyl-piperidin-4-yl)-cyclopropanecarboxylic acid (352 mg, 1.67 mmol), 5-(3,5-Difluoro-phenyl)-pyridin-2-ylamine (378 mg, 1.83 mmol), N-(3-dimethylaminopropyl)-N′-ethylcarbodiimide hydrochloride (479 mg, 2.50 mmol) and 4-dimethylaminopyridine (20.4 mg, 0.1 mmol) in methylene chloride (25 mL) was stirred at room temperature overnight. The mixture was concentrated in vacuo. The residue was purified by HPLC to afford 11-(1-Acetyl-piperidin-4-yl)-cyclopropanecarboxylic acid [5-(3,5-difluoro-phenyl)-pyridin-2-yl]-amide (40 mg, 6%). ¹H NMR (400 MHz, CDCl₃) δ 8.45-8.44 (dd, J=2.4 and 0.6 Hz, 1H), 8.28 (dd, J=8.6 and 0.6 Hz, 1H), 8.03 (s, 1H), 7.87 (dd, J=8.6 and 2.4 Hz, 1H), 7.10-7.04 (m, 2H), 6.86-6.80 (m, 1H), 4.80-4.71 (m, 1H), 3.89-3.80 (m, 1H), 3.04 (dt, J=12.0 and 2.2 Hz, 1H), 2.49 (dt, J=12.0 and 2.2 Hz, 1H), 2.08 (s, 3H), 1.86-1.74 (m, 3H), 1.44-1.31 (m, 2H), 1.14-1.08 (m, 2H), 0.87-0.81 (m, 2H). ESI-MS m/z: 400.0 (M+H)⁺.

Likewise, the following compound was prepared analogously to that of Example 4h.

### Example 4i

### 1-(1-Acetyl-piperidin-4-yl)-cyclopropanecarboxylic acid (3′,5′-difluoro-biphenyl-4-yl)-amide

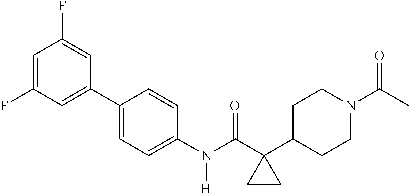

Prepared from 1-(1-acetyl-piperidin-4-yl)-cyclopropanecarboxylic acid and 3′,5′-difluoro-biphenyl-4-ylamine. LC-MS (m/z) 399.0 (MH⁺); t_{R}=1.26 min (Method A).

### Preparation of the Compounds of Formula I

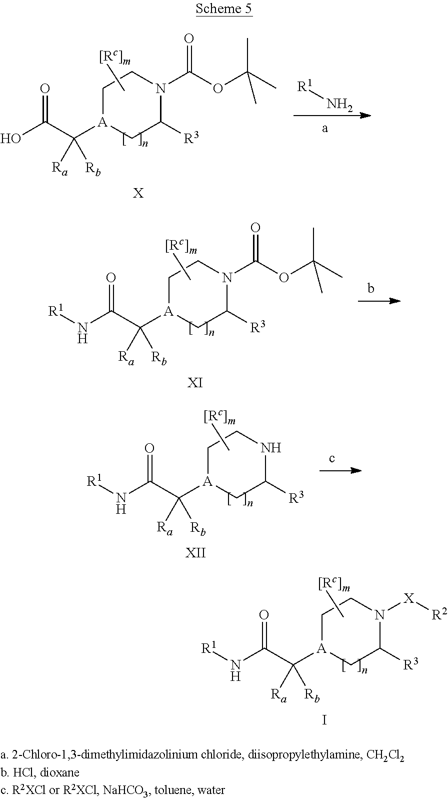

Representative intermediates were synthesized according to Scheme 5

### Intermediate of Formula XI: 4-(Biphenyl-4-ylcarbamoylmethyl)-piperidine-1-carboxylic acid tert-butyl ester

4-Carboxymethyl-piperidine-1-carboxylic acid tert-butyl ester (250 mg, 1.03 mmol) was dissolved in methylene chloride (5 mL) at room temperature. Diisopropylethylamine (0.54 mL, 3.09 mmol) was added followed by 2-chloro-1,3-dimethylimidazolinium chloride (174 mg, 1.03 mmol). The reaction mixture was stirred at room temperature for 10 minutes. 4-Biphenylaniline (174 mg, 1.03 mmol) was added and the reaction mixture was stirred overnight. The reaction mixture was diluted with ethyl acetate (25 mL), transferred to a separatory funnel, and washed with 1N HCl (1×10 mL) followed by aqueous saturated sodium bicarbonate (1×10 mL). The organic phase was dried over magnesium sulfate, filtered, and concentrated. The crude reaction mixture was dissolved in ethyl acetate (1 mL) and applied to a 5 g SCX cartridge (Supelco product number 52691-U). Elution with ethyl acetate followed by concentration afforded 162 mg of the title compound as a white solid. ¹H NMR (400 MHz, CDCl₃) δ 7.53-7.49 (m, 5H), 7.36 (t, J=6.1 Hz, 2H), 7.26 (t, J=6.1 Hz), 7.20-7.17 (m, 2H), 4.04 (br d, J=12.2 Hz, 2H), 2.68 (br t, J=12.3 Hz, 2H), 2.22 (br d, J=7.4 Hz, 2H), 2.07-1.98 (m, 2H), 1.71 (br d, J=11.5 Hz, 2H), 1.39 (s, 9H), 1.21-1.08 (m, 1H). ESI-MS m/z: 395.1 (M+H)⁺.

### Intermediate of Formula XII: N-Biphenyl-4-yl-2-piperidin-4-yl-acetamide

4-(Biphenyl-4-ylcarbamoylmethyl)-piperidine-1-carboxylic acid tert-butyl ester (152 mg, 0.39 mmol) was dissolved in dioxane (1 mL) at room temperature. A solution of HCl in dioxane (4M, 5 mL) was added and the reaction mixture was stirred 45 minutes at room temperature. The resulting solid was collected by filtration and triturated from ether to afford 114 mg of the title compound as a yellow-white solid. ¹H NMR (400 MHz, d-6 DMSO) δ 10.20 (s, 1H), 8.83 (br d, J=8.3 Hz, 1H), 8.65 (br d, J=8.3, 1H), 7.71 (d, J=8.7, 2H), 7.63 (t, J=8.1 Hz, 4H), 7.44 (t, J=5.6 Hz, 2H), 7.33 (t, J=9 Hz, 1H), 3.49 (br s, 2H), 3.24 (br d, J=13.1 Hz, 2H), 2.88 (br q, J=12.1 Hz, 2H), 2.33 (d, J=6.7 Hz, 2H), 2.12-2.02 (m, 1H), 1.83 (br d, J=13.4 Hz, 2H), 1.44 (br q, J=12.2 Hz, 2H). ESI-MS m/z: 295.1 (M+H)⁺.

### Example 5a

### 2-(1-Acetyl-piperidin-4-yl)-N-biphenyl-4-yl-acetamide

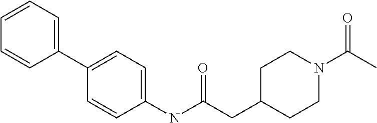

To a solution of N-biphenyl-4-yl-2-piperidin-4-yl-acetamide.HCl (15 mg, 0.05 mmol) in methylene chloride (0.5 mL) at room temperature was added triethylamine (0.1 mL, 0.7 mmol) followed by acetyl chloride (0.05 mL, 0.73 mmol). After stirring 10 minutes at room temperature, the reaction mixture was placed on a 1 g silica SPE cartridge. Elution with ethyl acetate followed by evaporation of the solvent afforded 10 mg of the title compound. NMR (400 MHz, CDCl₃) δ 8.02 (br s, 1H), 7.58 (br d, J=8.0 Hz, 2H), 7.50-7.47 (m, 4H), 7.35 (t, J=7.3 Hz, 2H), 7.25 (t, J=7.3 Hz, 1H), 4.56 (br s, 1H), 3.74 (br s, 1H), 3.03 (br s, 1H), 2.53 (br s, 1H), 2.24 (br s, 2H), 2.17 (m, 1H), 1.97 (2, 3H), 1.79 (br s, 2H), 1.13 (br s, 2H). LC-MS (m/z) 337.1 (MH⁺); t_{R}=1.18 min (Method B).

The following compounds were made in an analogous manner as above:

### Example 5b

### Acetic acid 2-[4-(biphenyl-3-ylcarbamoylmethyl)-piperidin-1-yl]-2-oxo-ethyl ester

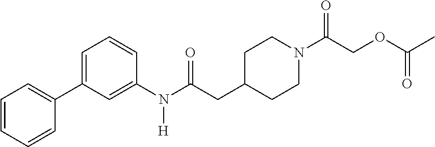

Prepared from N-biphenyl-3-yl-2-piperidin-4-yl-acetamide and acetoxyacetyl chloride. LC-MS (m/z) 395.1 (MH⁺); t_{R}=1.23 min (Method B)

### Example 5c

### 2-(1-Acetyl-piperidin-4-yl)-N-biphenyl-3-yl-acetamide

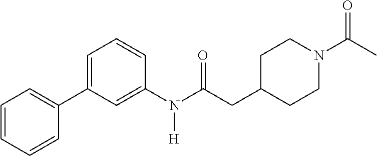

Prepared from N-biphenyl-3-yl-2-piperidin-4-yl-acetamide and acetyl chloride. LC-MS (m/z) 337.1 (MH⁺); t_{R}=1.18 min (Method B).

### Example 5d

### N-Biphenyl-4-yl-2-(1-propionyl-piperidin-4-yl)-acetamide

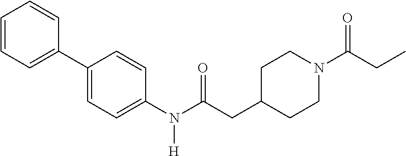

Prepared from N-biphenyl-4-yl-2-piperidin-4-yl-acetamide and propionyl chloride. LC-MS (m/z) 351.1 (MH⁺); t_{R}=1.27 min (Method B).

### Example 5e

### N-Biphenyl-4-yl-2-(1-isobutyryl-piperidin-4-yl)-acetamide

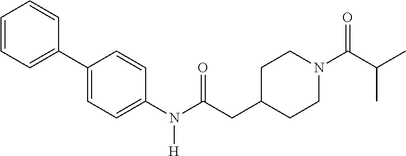

Prepared from N-biphenyl-4-yl-2-piperidin-4-yl-acetamide and isobutyryl chloride. LC-MS (m/z) 365.1 (MH⁺); t_{R}=1.35 min (Method B).

### Example 5f

### N-Biphenyl-4-yl-2-(1-cyclopropanecarbonyl-piperidin-4-yl)-acetamide

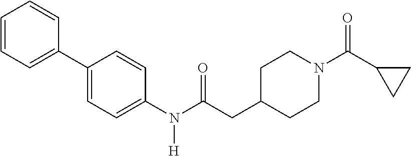

Prepared from N-biphenyl-4-yl-2-piperidin-4-yl-acetamide and cyclopropanecarbonyl chloride. LC-MS (m/z) 363.1 (MH⁺); t_{R}=1.31 min (Method B).

### Example 5g

### Acetic acid 2-[4-(biphenyl-4-ylcarbamoylmethyl)-piperidin-1-yl]-2-oxo-ethyl ester

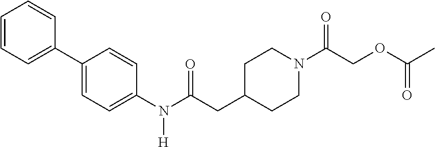

Prepared from N-biphenyl-4-yl-2-piperidin-4-yl-acetamide and acetoxyacetyl chloride. LC-MS (m/z) 395.1 (MH⁺); t_{R}=1.22 min (Method B).

### Example 5h

### N-Biphenyl-4-yl-2-[1-(3,3,3-trifluoro-propionyl)-piperidin-4-yl]-acetamide

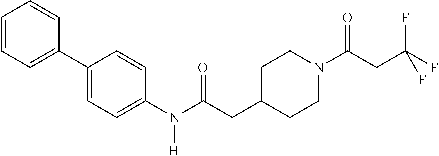

Prepared from N-biphenyl-4-yl-2-piperidin-4-yl-acetamide and 3,3,3-trifluoro-propionyl chloride. LC-MS (m/z) 405.0 (MH⁺); t_{R}=1.38 min (Method B).

### Example 5i

### 1-[1-(Pyridine-4-carbonyl)-piperidin-4-yl]-cyclopropanecarboxylic acid (3,5-difluoro-biphenyl-4-yl)-amide

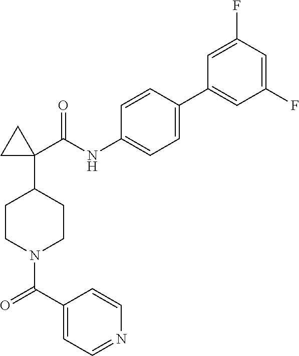

Prepared from 1-piperidin-4-yl-cyclopropanecarboxylic acid (3′,5′-difluoro-biphenyl-4-yl)-amide and isonicotinoyl chloride hydrochloride. ¹H NMR (300 MHz, CDCl₃) δ 8.69 (brs, 2H), 7.62-7.49 (m, 4H), 7.33-7.26 (m, 3H), 7.12-7.02 (m, 2H), 6.77 (tt, J=8.9 and 2.3 Hz, 1H), 4.82 (d, J=12.0 Hz, 1H), 3.67 (d, J=12.2 Hz, 1H), 3.01 (t, J=12.8 Hz, 1H), 2.72 (t, J=12.1 Hz, 1H), 1.92-1.69 (m, 3H), 1.61-1.45 (m, 2H), 1.07 (m, 2H), 0.83 (m, 2H). LC-MS (m/z) 462.0 (MH⁺); t_{R}=1.27 min (Method A).

### Example 5j

### 1-(1-Cyclobutanecarbonyl-piperidin-4-yl)-cyclopropanecarboxylic acid (3,5-difluoro-biphenyl-4-yl)-amide

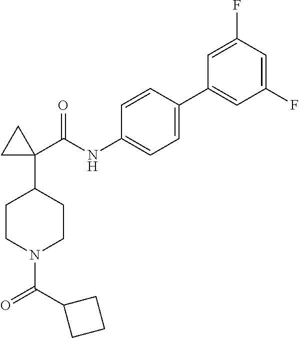

Prepared from 1-piperidin-4-yl-cyclopropanecarboxylic acid (3′,5′-difluoro-biphenyl-4-yl)-amide and cyclobutanecarbonyl chloride. ¹H NMR (300 MHz, CDCl₃) δ 7.60-7.55 (m, 2H), 7.52-7.48 (m, 2H), 7.33 (brs, 1H), 7.10-7.02 (m, 2H), 6.79-6.72 (tt, J=8.9 and 2.3 Hz, 1H), 4.72-4.63 (m, 1H), 3.78-3.73 (m, 1H), 3.23 (m, 1H), 2.95-2.85 (m, 1H), 2.53-2.43 (m, 1H), 2.39-2.24 (m, 2H), 2.18-2.06 (m, 2H), 1.98-1.71 (m, 5H), 1.39-1.23 (m, 2H), 1.03 (m, 2H), 0.80 (m, 2H). LC-MS (m/z) 439.0 (MH⁺); t_{R}=1.53 min (Method A).

### Example 5k

### 1-(1-Cyclopentanecarbonyl-piperidin-4-yl)-cyclopropanecarboxylic acid (3,5-difluoro-biphenyl-4-yl)-amide

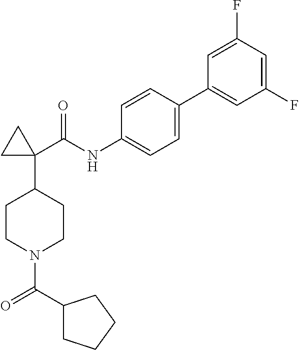

Prepared from 1-piperidin-4-yl-cyclopropanecarboxylic acid (3′,5′-difluoro-biphenyl-4-yl)-amide and cyclopentanecarbonyl chloride. ¹H NMR (300 MHz, CDCl₃) δ 7.60-7.56 (m, 2H), 7.53-7.49 (m, 2H), 7.32 (brs, 1H), 7.11-7.03 (m, 2H), 6.80-6.72 (tt, J=8.9 and 2.3 Hz, 1H), 4.76 (d, J=13.1 Hz, 1H), 4.05 (d, J=13.6 Hz, 1H), 3.02-2.82 (m, 2H), 2.49 (t, J=10.5 Hz, 1H), 1.87-1.53 (m, 11H), 1.53-1.28 (m, 2H), 1.04 (m, 2H), 0.80 (m, 2H). LC-MS (m/z) 453.0 (MH⁺); t_{R}=1.61 min (Method A).

### Example 5l

### 1-(1-Cyclohexanecarbonyl-piperidin-4-yl)-cyclopropanecarboxylic acid (3,5-difluoro-biphenyl-4-yl)-amide

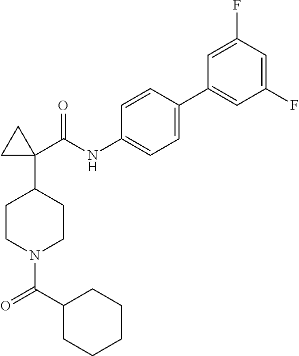

Prepared from 1-piperidin-4-yl-cyclopropanecarboxylic acid (3′,5′-difluoro-biphenyl-4-yl)-amide and cyclohexanecarbonyl chloride. ¹H NMR (300 MHz, CDCl₃) δ 7.60-7.56 (m, 2H), 7.52-7.49 (m, 2H), 7.29 (brs, 1H), 7.10-7.03 (m, 2H), 6.79-6.72 (tt, J=8.9 and 2.3 Hz, 1H), 4.75 (d, J=11.7 Hz, 1H), 3.99 (d, J=12.2 Hz, 1H), 2.98 (t, J=12.1 Hz, 1H), 2.50-2.41 (m, 3H), 1.89-1.22 (m, 14H), 1.04 (m, 2H), 0.80 (m, 2H). LC-MS (m/z) 467.0 (MH⁺); t_{R}=1.66 min (Method A)

### Example 5m

### 1-[1-(Pyridine-2-carbonyl)-piperidin-4-yl]-cyclopropanecarboxylic acid (3,5-difluoro-biphenyl-4-yl)-amide

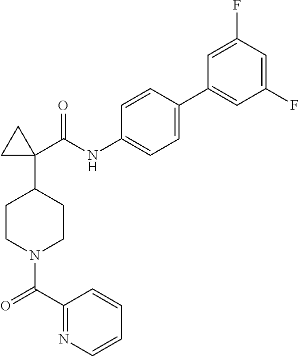

Prepared from 1-piperidin-4-yl-cyclopropanecarboxylic acid (3′,5′-difluoro-biphenyl-4-yl)-amide and picolinoyl chloride hydrochloride. ¹H NMR (300 MHz, CDCl₃) δ 8.58 (d, J=4.5 Hz 1H), 7.79 (dt, J=7.7 and 1.7 Hz, 1H), 7.61-7.57 (m, 3H), 7.52-7.48 (m, 2H), 7.37-7.31 (m, 2H), 7.11-7.03 (m, 2H), 6.76 (tt, J=8.9 and 2.3 Hz, 1H), 4.85 (d, J=12.8 Hz, 1H), 3.98 (d, J=13.5 Hz, 1H), 3.06 (t, J=12.5 Hz, 1H), 2.77 (t, J=10.7 Hz, 1H), 1.93-1.84 (m, 2H), 1.75-1.70 (m, 1H), 1.59-1.44 (m, 2H), 1.05 (m, 2H), 0.84 (m, 2H). LC-MS (m/z) 462.0 (MH⁺); t_{R}=1.32 min (Method A).

### Example 5n

### 1-[1-(3-Cyano-benzoyl)-piperidin-4-yl]-cyclopropanecarboxylic acid (3,5-difluoro-biphenyl-4-yl)-amide

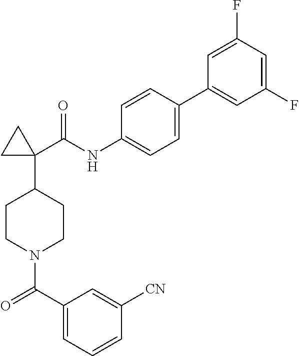

Prepared from 1-piperidin-4-yl-cyclopropanecarboxylic acid (3′,5′-difluoro-biphenyl-4-yl)-amide and 3-cyanobenzoyl chloride. ¹H NMR (300 MHz, CDCl₃) δ 7.72-7.67 (m, 2H), 7.65-7.61 (dt, J=7.9 and 1.4 Hz, 1H), 7.60-7.48 (m, 5H), 7.31 (brs, 1H), 7.11-7.02 (m, 2H), 6.76 (tt, J=8.9 and 2.3 Hz, 1H), 4.91 (brs, 1H), 3.70 (brs, 1H), 3.04 (brs, 1H), 2.73 (brs, 1H), 1.87-1.73 (m, 3H), 1.50 (brs, 2H), 1.07 (m, 2H), 0.84 (m, 2H). LC-MS (m/z) 486.0 (MH⁺); t_{R}=1.48 min (Method A).

### Example 5o

### 1-[1-(2-Trifluoromethyl-benzoyl)-piperidin-4-yl]-cyclopropanecarboxylic acid (3,5-difluoro-biphenyl-4-yl)-amide

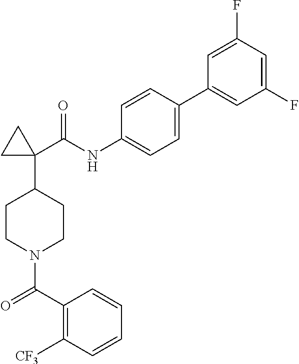

Prepared from 1-piperidin-4-yl-cyclopropanecarboxylic acid (3′,5′-difluoro-biphenyl-4-yl)-amide and 2-trifluoromethylbenzoyl chloride. ¹H NMR (300 MHz, CDCl₃) δ 7.74-7.66 (m, 1H), 7.60-7.47 (m, 6H), 7.40-7.26 (m, 2H), 7.10-7.02 (m, 2H), 6.80-6.71 (m, 1H), 4.92-4.82 (m, 1H), 3.51-3.31 (m, 1H), 3.07-2.87 (m, 1H), 2.78-2.65 (m, 1H), 1.91-1.23 (m, 5H), 1.04 (m, 2H), 0.80 (m, 2H). LC-MS (m/z) 529.0 (MH⁺); t_{R}=1.62 min (Method A).

### Example 5p

### 1-[1-(Pyrazine-2-carbonyl)-piperidin-4-yl]-cyclopropanecarboxylic acid (3,5-difluoro-biphenyl-4-yl)-amide

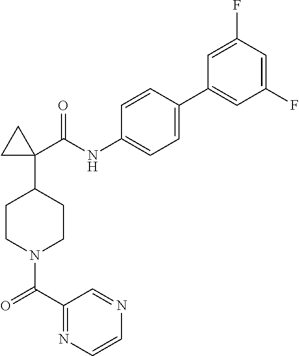

Prepared from 1-piperidin-4-yl-cyclopropanecarboxylic acid (3′,5′-difluoro-biphenyl-4-yl)-amide and pyrazine-2-carbonyl chloride. ¹H NMR (300 MHz, CDCl₃) δ 8.90 (d, J=1.5 Hz, 1H), 8.62 (d, J=2.5 Hz, 1H), 8.53 (dd, J=2.5 and 1.5 Hz, 1H), 7.61-7.56 (dt, J=8.7 and 2.4 Hz, 2H), 7.52-7.48 (dt, J=8.7 and 2.3 Hz, 2H), 7.34 (brs, 1H), 7.09-7.03 (m, 2H), 6.78-6.72 (tt, J=8.8 and 2.3 Hz, 1H), 4.87-4.82 (m, 1H), 4.05-4.00 (m, 1H), 3.12-3.05 (dt, J=13.0 and 2.5 Hz, 1H), 2.81-2.73 (dt, J=13.0 and 2.6 Hz, 1H), 1.94-1.86 (m, 2H), 1.80-1.72 (m, 1H), 1.59-1.47 (m, 2H), 1.05 (m, 2H), 0.84 (m, 2H). LC-MS (m/z) 462.9 (MH⁺); t_{R}=1.28 min (Method A).

### Example 5q

### N-[5-(3,5-Difluoro-phenyl)-pyridin-2-yl]-2-[1-(2-methyl-2H-pyrazole-3-carbonyl)-piperidin-4-yl]-isobutyramide

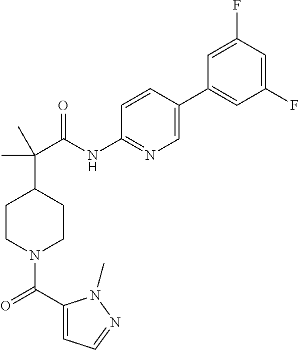

Prepared from N-(3′,5′-Difluoro-biphenyl-4-yl)-2-piperidin-4-yl-isobutyramide and 2-methyl-2H-pyrazole-3-carbonyl chloride. ¹H NMR (400 MHz, CDCl₃) δ 8.46 (dd, J=2.4 and 0.6 Hz, 1H), 8.35 (dd, J=8.8 and 0.6 Hz, 1H), 8.16 (brs, 1H), 7.89 (dd, J=8.7 and 2.5 Hz, 1H), 7.43 (d, J=2.0 Hz, 1H), 7.10-7.04 (m, 2H), 6.83 (tt, J=8.8 and 2.3 Hz, 1H), 6.29 (d, J=2.0 Hz, 1H), 4.80 (brs, 1H), 4.09 (brs, 1H), 3.96 (s, 3H), 3.06 (brs, 1H), 2.73 (brs, 1H), 2.04 (tt, J=12.2 and 2.8 Hz, 1H), 1.70 (m, 2H), 1.36-1.25 (m, 2H), 1.30 (s, 6H). LC-MS (m/z) 467.9 (MH⁺); t_{R}=1.33 min (Method A).

### Example 5r

### N-[5-(3,5-Difluoro-phenyl)-pyridin-2-yl]-2-[1-(isoxazole-5-carbonyl)-piperidin-4-yl]-isobutyramide

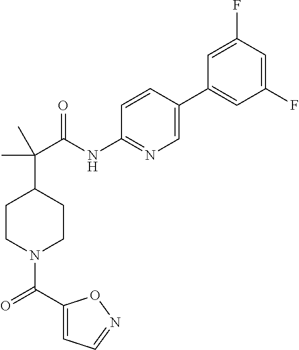

Prepared from N-(3′,5′-Difluoro-biphenyl-4-yl)-2-piperidin-4-yl-isobutyramide and isoxazole-5-carbonyl chloride. ¹H NMR (300 MHz, CDCl₃) δ 8.46 (d, J=2.5 Hz, 1H), 8.35 (d, J=8.7 Hz, 1H), 8.30 (d, J=1.9 Hz, 1H), 8.14 (brs, 1H), 7.88 (dd, J=8.8 and 2.6 Hz, 1H), 7.09-7.05 (m, 2H), 6.86-6.80 (m, 1H), 6.74 (d, J=1.7 Hz, 1H), 4.75 (m, 1H), 4.26 (m, 1H), 3.13 (m, 1H), 2.79 (m, 1H), 2.07 (m, 1H), 1.82-1.37 (m, 4H), 1.30 (s, 6H). LC-MS (m/z) 454.9 (MH⁺); t_{R}=1.39 min (Method A).

### Example 5s

### N-[5-(3,5-Difluoro-phenyl)-pyridin-2-yl]-2-[1-(thiazole-2-carbonyl)-piperidin-4-yl]-isobutyramide

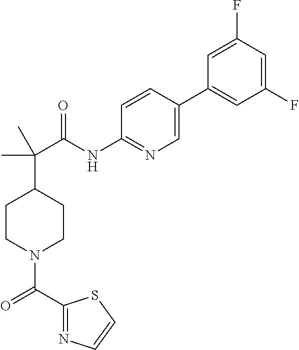

Prepared from N-(3′,5′-Difluoro-biphenyl-4-yl)-2-piperidin-4-yl-isobutyramide and 1,3-thiazole-2-carbonyl chloride. ¹H NMR (300 MHz, CDCl₃) δ 8.46 (d, J=2.3 Hz, 1H), 8.36 (d, J=8.8 Hz, 1H), 8.13 (brs, 1H), 7.88 (dd, J=8.5 and 2.2 Hz, 1H), 7.86 (d, J=3.2 Hz, 1H), 7.51 (d, J=3.2 Hz, 1H), 7.09-7.05 (m, 2H), 6.83 (tt, J=8.6 and 2.3 Hz, 1H), 5.59 (d, J=12.3 Hz, 1H), 4.82 (d, J=11.4 Hz, 1H), 3.10 (t, J=13.9 Hz, 1H), 2.79 (t, J=12.2 Hz, 1H), 2.07 (m, 1H), 1.81-1.43 (m, 4H), 1.30 (s, 6H). LC-MS (m/z) 470.8 (MH⁺); t_{R}=1.52 min (Method A).

### Example 5t

### N-[5-(3,5-Difluoro-phenyl)-pyridin-2-yl]-2-[1-(1H-pyrrole-2-carbonyl)-piperidin-4-yl]-isobutyramide

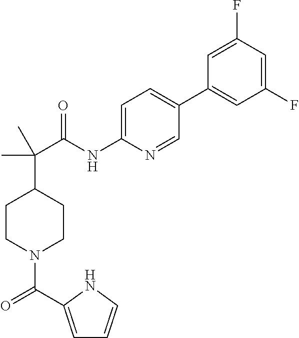

Prepared from N-(3′,5′-Difluoro-biphenyl-4-yl)-2-piperidin-4-yl-isobutyramide and 1H-pyrrole-2-carboxylic acid. ¹H NMR (400 MHz, CDCl₃) δ 9.40 (brs, 1H), 8.46 (dd, J=2.5 and 0.6 Hz, 1H), 8.36 (dd, J=8.7 and 0.6 Hz, 1H), 8.17 (brs, 1H), 7.89 (dd, J=8.6 and 2.5 Hz, 1H), 7.10-7.04 (m, 2H), 6.90 (dt, J=2.6 and 1.2 Hz, 1H), 6.83 (tt, J=8.8 and 2.3 Hz, 1H), 6.50 (m, 1H), 6.23 (m, 1H), 4.72 (d, J=13.1 Hz, 2H), 2.94 (brs, 2H), 2.05 (m, 1H), 1.74 (d, J=12.4 Hz, 2H), 1.45-1.35 (m, 2H), 1.29 (s, 6H). LC-MS (m/z) 452.9 (MH⁺); t_{R}=1.44 min (Method A).

### Example 5u

### N-[5-(3,5-Difluoro-phenyl)-pyridin-2-yl]-2-[1-(1H-imidazole-4-carbonyl)-piperidin-4-yl]-isobutyramide

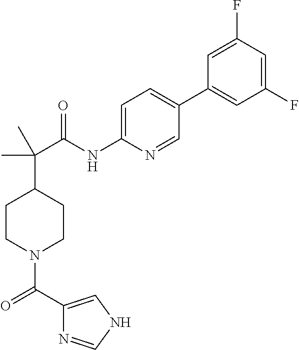

Prepared from N-(3′,5′-Difluoro-biphenyl-4-yl)-2-piperidin-4-yl-isobutyramide and 1H-imidazole-4-carboxylic acid. ¹H NMR (400 MHz, CDCl₃) δ 8.46 (dd, J=2.5 and 0.6 Hz, 1H), 8.36 (dd, J=8.7 and 0.6 Hz, 1H), 8.23 (brs, 1H), 7.89 (dd, J=8.7 and 2.5 Hz, 1H), 7.46 (dd, J=1.7 and 0.8 Hz, 1H), 7.11-7.04 (m, 2H), 6.96 (dd, J=3.5 and 0.8 Hz, 1H), 6.83 (tt, J=8.8 and 2.3 Hz, 1H), 6.46 (dd, J=3.4 and 1.8 Hz, 1H), 4.65 (brs, 2H), 2.87 (m, 2H), 2.04 (tt, J=12.3 and 3.3 Hz, 1H), 1.74 (d, J=12.7 Hz, 2H), 1.47-1.34 (m, 2H), 1.29 (s, 6H). LC-MS (m/z) 454.0 (MH⁺); t_{R}=1.14 min (Method A).

### Example 5v

### N-[5-(3,5-Difluoro-phenyl)-pyridin-2-yl]-2-[1-(furan-2-carbonyl)-piperidin-4-yl]-isobutyramide

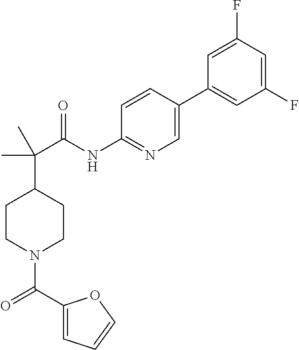

Prepared from N-(3′,5′-Difluoro-biphenyl-4-yl)-2-piperidin-4-yl-isobutyramide and furan-2-carboxylic acid. LC-MS (m/z) 453.8 (MH⁺); t_{R}=1.46 min (Method A).

### Example 5w

### N-[5-(3,5-Difluoro-phenyl)-pyridin-2-yl]-2-[1-(pyridine-2-carbonyl)-piperidin-4-yl]-isobutyramide

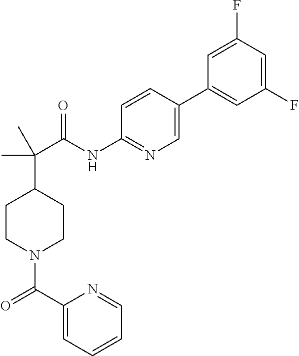

Prepared from N-(3′,5′-Difluoro-biphenyl-4-yl)-2-piperidin-4-yl-isobutyramide and picolinoyl chloride hydrochloride. ¹H NMR (300 MHz, CDCl₃) δ 8.57 (d, J=4.7 Hz, 1H), 8.46 (d, J=2.2 Hz, 1H), 8.35 (d, J=8.7 Hz, 1H), 8.11 (brs, 1H), 7.87 (dd, J=8.7 and 2.4 Hz, 1H), 7.78 (dt, J=7.7 and 1.7 Hz, 1H), 7.60 (d, J=7.8 Hz, 1H), 7.32 (m, 1H), 7.10-7.03 (m, 2H), 6.82 (tt, J=8.8 and 2.3 Hz, 1H), 4.86 (d, J=13.1 Hz, 1H), 4.02 (d, J=11.2 Hz, 1H), 3.05 (m, 1H), 2.77 (m, 1H), 2.03 (m, 1H), 1.81-1.37 (m, 4H), 1.29 (d, J=5.4 Hz, 6H). LC-MS (m/z) 464.9 (MH⁺); t_{R}=1.31 min (Method A).

The following advanced intermediates which were used in the above-identified schemes above can be synthesized as follows:
Hexahydro-pyrrolo[1,2-a]pyrazin-6-one and its stereoisomers can be prepared according to Christensen, et al. WO 08/46882;
1-((S)-2-Methyl-piperazin-1-yl)-ethanone and 1-((R)-2-Methyl-piperazin-1-yl)-ethanone can be prepared according to Shima, et al. WO 98/35951;
1-((R)-3-Methyl-piperazin-1-yl)-ethanone and 1-((S)-3-methyl-piperazin-1-yl)-ethanone can be prepared according to Manetti, et. al. J. Med. Chem., 2000, 43, 4499-4507;
(S)-Hexahydro-1-thia-5,7a-diaza-indene 1,1-dioxide can be prepared according to Alvaro et al., WO 07/028,654;
(1-Acetyl-piperidin-4-yl)-acetic acid can be prepared according to Orjales et. al. J. Med. Chem., 2003, 46, 5512-5532;
(1-Methanesulfonyl-piperidin-4-yl)-acetic acid can be prepared according to Tsuchimori et. al. WO 03/090748; and
4-Carboxymethyl-4-methyl-piperidine-1-carboxylic acid tert-butylester can be prepared according to Faull, et al. WO 2006/0017152.

### Example 6

### In-Vitro Pharmacological Evaluation at Cloned Neuropeptide Y5 Receptor

The pharmacological properties of the compounds of the present invention were evaluated at the cloned human NPY Y5 receptor using the protocols disclosed in U.S. Pat. No. 6,124,331, the contents of which are hereby incorporated by reference, or in an assay as similarly described therein for NPY Y5 binding activity. The procedures for cell culture, transient transfection and membrane harvest, which are well known in the art, are described therein

### Radioligand Binding to Membrane Suspensions

Briefly, membrane suspensions from transfected cells (typically expressed in LM(tk-) cells) and ¹²⁵I-PYY radioligand (PerkinElmer, Waltham, Mass.) were diluted in binding buffer supplemented with 0.1% bovine serum albumin to yield an optimal membrane protein concentration so that ¹²⁵I-PYY bound by membranes in the assay was less than 10% of ¹²⁵I-PYY delivered to the sample (100,000 dpm/sample=0.08 nM for competition binding assays). Test compounds were diluted to desired concentrations with supplemented binding buffer in the presence of 30% DMSO. The binding assay was performed in 96-well polypropylene microtiter plates by mixing ¹²⁵I-PYY, test compound (25 μL), and finally, membrane suspensions (200 μL). Final DMSO=3%. Samples were incubated in at room temperature for about 120 min. Incubations were terminated by filtration over Whatman GF/C filters (pre-coated with 1% polyethyleneimine and air-dried before use), followed by washing with ice-cold binding buffer. Filter-trapped membranes were impregnated with MeltiLex solid scintillant (Wallac, Turku, Finland) and counted for ¹²⁵I-PYY in a Wallac MicroBeta Trilux. Non-specific binding was defined by 1000 nM porcine NPY. Specific binding was typically 80%; most non-specific binding was associated with the filter. Binding data were analyzed using nonlinear regression and statistical techniques available in the GraphPAD Prism package (San Diego, Calif.).

### Radioligand Binding Assay Results

The binding affinities for the compounds in the present invention, exemplified above, at the human NPY Y5 receptor were determined to be 10 μM or less. The binding affinities for most of the compounds were determined to be 1.0 μM or less. The binding affinities for several compounds were determined to be 100 nM or less.

TABLE I
binding affinity of selected compounds

| Example No. | Ki (nM) |
|---|---|
| 1a | 220 |
| 1e | 120 |
| 2a | 46 |
| 2aj | 28 |
| 3a | 36 |
| 3i | 50 |
| 3l | 25 |
| 3o | 17 |
| 3cc | 8 |
| 3hh | 39 |
| 4a | 66 |
| 5a | 120 |
| 5j | 2.7 |
| 5m | 14 |
| 5q | 570 |

### Functional Assay: NPY Y5-Dependent Inhibition of Forskolin-Stimulated Camp Accumulation

Stably transfected cells were seeded into 96-well microtiter plates and cultured until confluent. To reduce the potential for receptor desensitization, the serum component of the media was reduced to 1.5% for 4 to 16 hours before the assay. Cells were washed in Hank's buffered saline, or HBS (150 mM NaCl, 20 mM HEPES, 1 mM CaCl₂, 5 mM KCl, 1 mM MgCl₂, and 10 mM glucose) supplemented with 0.1% bovine serum albumin plus 100 uM IBMX. Test compounds were diluted to desired concentrations with assay buffer in the presence of 10% DMSO, then transferred to the cell plate and allowed to incubate for 20 min at 37° C. in 5% CO₂ (final DMSO=1%). Cells were then stimulated with NPY (up to 10 uM) for a period of 5 min, followed by forskolin (10 uM) for another 5 min. The assay was then terminated and intracellular cAMP was quantified by with highthroughput time-resolved fluorometry (HTRF kit from CisBio, Bedford, Mass.). The effect of the test compound on agonist (NPY) activity were analyzed using nonlinear regression and statistical techniques available in the GraphPAD Prism package (San Diego, Calif.).

### Functional Assay Results

The compounds of Examples 2a and 4a were determined to function as antagonists at the NPY Y5 receptor. The Compounds of the Invention were selected and tested in an assay as described above or similarly described herein for NPY5 binding and functional activity.

### Example 7

### In-Vivo Assays

The in-vivo effects of the compounds of the present invention can be evaluated by using the following in-vivo behavioral animal models. The behavioral models described below are not intended to be the only models used to determine the efficacy of a compound of the invention to treat the corresponding disorder.

Agonist-stimulated feeding assay. Sprague-Dawley rats (250-275 g) are implanted with guide cannulae into the lateral cerebral ventricle at Charles River Laboratories (Kingston, N.Y.) and shipped to the animal facility 2-3 days later. After a 1 week acclimation period, successful cannulae placement was confirmed by robust drinking in response to an i.c.v. infusion of angiotensin II (100 μg). At least 5 days prior to initiation of feeding studies, rats are acclimated to cages with wire grid floors, suspended by a stainless steel support above a waste tray. Food was removed on the morning of testing. To study the blockade of Y₅ receptor-induced feeding by the compound, animals were dosed orally with vehicle alone (20% cyclodextrin in distilled H₂O) or the compound, followed 1 h later by i.c.v. infusion (5 μl over 1 min) of the Y₅ receptor-selective peptide agonist cPP (0.6 nmol in normal saline). The 0.6 nmol dose of cPP was selected because it is just below the ED₅₀ dose (0.75 nmol) determined from preliminary experiments (data not shown) and provides a robust feeding signal. Animals are returned to the feeding cages and a pre-weighed amount of food was made available for 1 h. Net food intake=pre-weighed amount−(final amount+spilled amount). To test for nonspecific inhibition of the feeding response, we assess the effect of the compound (30 mg/kg) on feeding induced by 2 nmol NPY, a dose that evoked a feeding response equivalent to that of 0.6 nmol cPP.

Rat Forced-swim Test: The procedure which may be used here is similar to that previously described (Luki, et al. Psychopharmacology 2001, 155, 315-322) with the following modifications. Male Sprague-Dawley rats may be used. Swim sessions are conducted for about 5 min, by placing rats in a plexiglass cylinder (about 46 cm tall×20 cm in diameter) filled about 30 cm deep with water at about 23° C. A compound of the invention or vehicle (about 0.01% lactic acid, about pH 6) is administered orally as a 1 ml/kg solution. Test sessions are videotaped and recorded for later scoring by a single rater, who is blinded to the treatment condition. Immobility is scored as the time a rat remained floating in the water making only movements necessary to keep its head above the water. Swimming is scored as the time a rat made active swimming motions, more than necessary to maintain its head above water.

Rat Social-interaction Test: The procedure is performed for about 15 min as previously described (File and Hyde Br. J. Pharmacol. 1987, 62, 19-24) under low-light conditions using pairs of unfamiliar male Sprague-Dawley rats previously housed singly and exposed to the test arena for about 15 min on the previous day. A compound of the invention, chlordiazepoxide or vehicle is injected i.p. as a ˜1.0 ml/kg solution. All test sessions are videotaped and recorded for later scoring. Active social interaction, defined as sniffing, grooming, biting, boxing and crawling over and under, as well as locomotor activity (defined as squares crossed), is scored by a single rater, who is blinded to the treatment of each pair.

Chronic mild stress. The chronic mild stress (CMS) test is performed using male Wistar rats as described previously (Papp et al., 2002). Rats are first trained to consume a 1% sucrose solution in a series of baseline tests during which the sucrose solution was presented in the home cage for 1 h following 14 h food and water deprivation. On the basis of their final sucrose intake scores, animals are divided into two matched groups, one subjected continuously to chronic mild stress for a period of 8-9 consecutive weeks and the other housed separately as a non-stressed control group. Both groups are subjected to a sucrose consumption test once weekly, around 10:00 a.m. and under similar conditions as in the training period. Following 2-3 weeks of stress, sucrose intake scores are used to further divide both stressed and control animals into matched groups (n=8 per group). For the next 5 weeks both stressed and control animals receive twice daily intraperitoneal injections of vehicle (0.25% hydroxypropyl β methylcellulose, 1 ml/kg), a compound of the invention or citalopram at approx. 10:00 and 17:00, except that the 17:00 i.p. injection is omitted on days preceding the sucrose test. After 5 weeks the i.p. injections are terminated in both groups. Twenty-four h after the last i.p. injection, all animals are sacrificed and brains are removed.

1. A compound of Formula I: 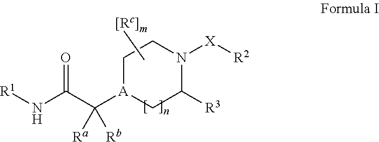
wherein R¹ is phenyl, pyrazoyl, imidazoyl, isoxazoyl, oxadiazoyl, thiadiazoyl, pyridyl, pyrimidyl or pyrazinyl, where the phenyl, pyrazoyl, imidazoyl, isoxazoyl, oxadiazoyl, thiadiazoyl, pyridyl, pyrimidyl and pyrazinyl are optionally substituted with one or more R⁴;
wherein R² is C₁-C₇ alkyl, C₁-C₇ alkoxy, NH(C₁-C₇ alkyl), (CH₂)ᵥOC(O)C₁-C₇ alkyl, C₃-C₇ cycloalkyl, phenyl, pyridyl, pyrimidinyl, pyrazinyl, triazoyl, tetrazolyl, thiazoyl, oxazoyl, imidazoyl, isoxazoyl, furanyl or pyrazoyl, where the phenyl, pyridyl, pyrimidinyl, pyrazinyl, triazoyl, tetrazolyl, thiazoyl, oxazoyl, imidazoyl, isoxazoyl, furanyl and pyrazoyl are optionally substituted with one or more R⁵;
wherein R³ is H or C₁-C₇ alkyl, or where R³ can combine with R² to form C₁-C₄ alkylene optionally substituted with C₁-C₄ alkyl;
wherein R⁴ is C₁-C₇ alkyl, C₁-C₇ perfluoroalkyl, C(O)C₁-C₇ alkyl or phenyl optionally substituted with one or more C₁-C₇ alkyl, C₁-C₇ perfluoroalkyl, C₁-C₇ alkoxy or halogen;
wherein R⁵ is C₁-C₇ alkyl, C₁-C₇ perfluoroalkyl, C₁-C₇ alkoxy or halogen;
wherein A is CH, COH or N;
wherein X is C(O), CO₂ or S(O)₂;
wherein each R^{a} and R^{b} is independently H or C₁-C₇ alkyl, or where R^{a} and R^{b} can combine to form C₃-C₇ cycloalkyl;
wherein each R^{c} is independently H or C₁-C₇ alkyl;
wherein each m and v is independently an integer from 1 to 4 inclusive; and
wherein n is an integer from 0 to 2 inclusive; or a pharmaceutically acceptable salt thereof.

2. The compound of claim 1, wherein X is C(O).

3. The compound of claim 1, wherein X is CO₂.

4. The compound of claim 1, wherein X is S(O)₂.

5. The compound of claim 1, wherein R¹ is phenyl optionally substituted with one or more R⁴.

6. The compound of claim 1, wherein R¹ is pyridyl optionally substituted with one or more R⁴.

7. The compound of claim 1, wherein R¹ is [1,3]pyrazoyl optionally substituted with one or more R⁴.

8. The compound of claim 1, wherein R¹ is pyrimidinyl or pyrazinyl, where the pyrimidinyl and pyriazinyl are optionally substituted with one or more R⁴.

9. The compound of claim 1, wherein A is CH; and wherein n is 1.

10. The compound of claim 1, wherein A is N; and wherein n is 1 or 2.

11. The compound of claim 1, wherein R² is C₃-C₆ cycloalkyl; and wherein R³ is H or C₁-C₄ alkyl.

12. The compound of claim 1, wherein R² is C₁-C₇ alkoxy, NH(C₁-C₄ alkyl), or (CH₂)ᵥC(O)C₁-C₄ alkyl; wherein R³ is H or C₁-C₄ alkyl; and wherein v is 1 or 2.

13. The compound of claim 1, wherein R² combines with R³ to form methylene.

14. The compound of claim 1, wherein R² is phenyl optionally substituted with one or more R⁵.

15. The compound of claim 1, wherein R² is pyridyl, pyrimidyl, pyrazinyl or triazinyl, where the pyridyl, pyrimidyl, pyrazinyl and triazinyl are optionally substituted with one or more R⁵.

16. The compound of claim 1, wherein R² is tetrazolyl, thiazoyl, oxazoyl, imidazoyl, isoxazoyl and pyrazoyl, where the tetrazolyl, thiazoyl, oxazoyl, imidazoyl, isoxazoyl and pyrazoyl are optionally substituted with one or more R⁵.

17. The compound of claim 1, wherein each R^{a} and R^{b} is independently H or C₁-C₄ alkyl; and wherein m is 0 or 1.

18. The compound of claim 1, wherein R^{a} and R^{b} combine to form C₃-C₇ cycloalkyl; and wherein m is 0 or 1.

19. The compound of claim 1, wherein each R^{c} is independently H or C₁-C₄ alkyl; and wherein m is 0 or 1.

20. The compound of claim 1, wherein R⁴ is phenyl optionally substituted with one or more C₁-C₄ alkyl, C₁-C₄ alkoxy, fluorine or chlorine.

21. The compound of claim 1, wherein R⁴ is C₁-C₄ alkyl or C₁-C₄ perfluoroalkyl.

22. The compound of claim 1, wherein R⁵ is C₁-C₄ alkyl, fluorine or chlorine.

23. A pharmaceutical composition comprising a therapeutically effective amount of the compound of claim 1 or a pharmaceutically acceptable salt thereof.

24. A method of treating a subject suffering from a cognitive disorder comprising administering a therapeutically effective amount of the compound of claim 1 or a pharmaceutically acceptable salt thereof, sufficient to treat said subject.
